(19) United States
(12) Patent Application Publication
(10) Pub. No.: US 2017/0267970 A1
(43) Pub. Date: Sep. 21, 2017

(54) Three-Dimensional Hydrogels that Support Growth of Physiologically Relevant Tissue and Methods of Use Thereof

(71) Applicant: Whitehead Institute for Biomedical Research, Cambridge, MA (US)

(72) Inventors: Piyush GUPTA, Boston, MA (US); Daniel MILLER, Somerville, MA (US); Ethan SOKOL, Somerville, MA (US)

(73) Assignee: Whitehead Institute for Biomedical Research, Cambridge, MA (US)

(21) Appl. No.: 15/444,413

(22) Filed: Feb. 28, 2017

Related U.S. Application Data
(60) Provisional application No. 62/301,151, filed on Feb. 29, 2016.

(51) Int. Cl. C12N 5/00; A61L 27/26; A61L 27/54; A01K 67/027; C12N 5/071; G01N 33/50; C12N 5/09; C12N 5/0793; A61L 27/52; A61L 27/38

(52) U.S. Cl. CPC C12N 5/0062; A61L 27/52; A61L 27/26; A61L 27/54; A61L 27/3813; A61L 27/383; C12N 5/0631; G01N 33/5082; G01N 33/5011; C12N 5/0693; C12N 5/0619; A01K 67/0271; A61L 2300/414; A61L 2300/252; C12N 2539/00; C12N 2533/52; C12N 2533/54; C12N 2533/80; C12N 2513/00; A01K 2207/12; A01K 2267/03

(57) ABSTRACT

The presently disclosed subject matter provides hydrogel precursor compositions (e.g., solutions) for forming three-dimensional hydrogels that support growth of physiologically relevant tissue when at least one cell is cultured in the three-dimensional hydrogel, kits comprising the hydrogel precursor composition, three-dimensional hydrogels, methods of forming the three-dimensional hydrogels, methods of growing the physiologically relevant tissue using the three-dimensional hydrogels, physiologically relevant tissue grown in the three-dimensional hydrogels, methods of producing hormone-responsive tissue (e.g., milk-producing mammary tissue and related methods of producing milk), methods of screening for candidate agents useful for modulating hormonal responses (e.g., modulating milk production), method of screening for candidate therapeutic agents using the physiologically relevant tissue grown in the three-dimensional hydrogels (e.g., personalized cancer treatments), and related methods of treatment (e.g., administering agents identified using the methods herein, transplanting physiologically relevant tissue produced using the methods, etc.).

### CROSS-REFERENCE TO RELATED APPLICATION

This application claims the benefit of U.S. Provisional Application No. 62/301,151, filed Feb. 29, 2016, which is incorporated herein by reference in its entirety.

### BACKGROUND

Three-dimensional cultures have contributed valuable insights into murine biology, such as murine mammary gland biology (Chen et al., 2014; Ewald et al., 2008; Lee et al., 1985; Lee et al., 1984; Simian et al., 2001; Sternlicht et al., 2005). Tissue of mice (e.g., mammary tissue), however, is known to differ in important ways from tissue in humans (Cardiff and Wellings, 1999; Visvader, 2009). In an effort to address this problem, several investigators have successfully grown tissues from human cell lines immortalized by transduction with viral oncogenes (e.g., mammary tissues have been grown from human mammary cell lines immortalized by transduction with viral oncogenes (Berdichevsky et al., 1994; Debnath et al., 2003; Gudjonsson et al., 2002)). However, growing tissues from primary human cells (e.g., primary human mammary cells) has proven to be more challenging. For example, Tanos and colleagues showed that they could maintain viable primary human mammary tissue fragments in liquid cultures for up to 6 days (Tanos et al., 2013), but this system did not support the initiation or elongation of ducts. Moreover, ductal growth is also limited in collagen or basement membrane (Matrigel) 3D cultures of primary human mammary tissue (Pasic et al., 2011; Yang et al., 1987). Thus, there exists a need for three-dimensional culture systems that support outgrowth of morphologically complex and hormone-responsive tissues (e.g., morphologically complex and hormone-responsive mammary tissue) from primary human cells (e.g., that have not been immortalized through extensive culture or viral infection).

### SUMMARY

In some aspects, the presently disclosed subject matter provides a hydrogel precursor composition (e.g., solution) for forming a three-dimensional hydrogel that supports growth of a physiologically relevant tissue when at least one cell is cultured in the three-dimensional hydrogel, the hydrogel precursor solution consisting of, consisting essentially of, or comprising: (a) an aqueous medium; (b) at least three hydrogel precursor components dissolved in the aqueous medium to form a hydrogel precursor solution for forming a three-dimensional hydrogel that supports growth of physiologically relevant tissue, wherein the at least three hydrogel precursor components comprise: (i) a first hydrogel precursor component comprising an extracellular matrix protein selected from the group consisting of collagen, fibronectin, and laminin; (ii) a second hydrogel precursor component comprising hyaluronan or a glycosaminoglycan having a water-chelating ability that is similar to hyaluronan; and (ii) a third hydrogel precursor component comprising at least one agent that promotes growth of a physiologically relevant tissue, wherein the hydrogel precursor solution polymerizes under suitable conditions to form a three-dimensional hydrogel that supports growth of a physiologically relevant tissue when at least one cell is cultured in the three-dimensional hydrogel.

In some aspects, the presently disclosed subject matter provides a kit for forming a three-dimensional hydrogel that supports growth of a physiologically relevant tissue when at least one cell is cultured in the three-dimensional hydrogel, the kit consisting of, consisting essentially of, or comprising: a presently disclosed hydrogel precursor composition (e.g., solution); and (b) instructions for polymerizing the hydrogel precursor solution to form a three-dimensional hydrogel that supports growth of a physiologically relevant tissue when at least one cell is cultured in the three-dimensional hydrogel.

In some aspects, the presently disclosed subject matter provides a method of forming a three-dimensional hydrogel that supports growth of a physiologically relevant tissue when at least one cell is cultured in the three-dimensional hydrogel, the method comprising: (a) providing a presently disclosed hydrogel precursor composition (e.g., solution) or presently disclosed kit; and (b) incubating the hydrogel precursor solution at an elevated temperature for a period of time sufficient for the hydrogel precursor solution to polymerize and form a three-dimensional hydrogel that supports growth of a physiologically relevant tissue when at least one cell is cultured in the three-dimensional hydrogel.

In some embodiments, the presently disclosed subject matter provides a three-dimensional hydrogel that supports growth of a physiologically relevant tissue when at least one cell is cultured in the three-dimensional hydrogel, the three-dimensional hydrogel consisting of, consisting essentially of, or comprising: (a) an extracellular matrix protein selected from the group consisting of collagen, fibronectin, and laminin; (b) hyaluronan or a glycosaminoglycan having a water-chelating ability that is similar to hyaluronan; and (c) at least one agent that promotes growth of a physiologically relevant tissue; and (d) at least one cell, wherein (a) and (b) are polymerized into a three-dimensional hydrogel and (c) and (d) are embedded in the three-dimensional hydrogel; and wherein the three-dimensional hydrogel supports growth of a physiologically relevant tissue when (d) is cultured in the three-dimensional hydrogel in the presence of (c).

In some aspects, the presently disclosed subject matter provides a method for growing a physiologically relevant tissue from at least one cell, the method consisting of, consisting essentially of, or comprising: (a) providing a presently disclosed three-dimensional hydrogel; (b) optionally providing a defined culture medium; and (c) culturing the at least one cell in the three-dimensional hydrogel, in the presence of the defined culture medium if provided, for a period of time sufficient for the at least one cell to grow into a physiologically relevant tissue or physiologically relevant component thereof.

In some aspects, the presently disclosed subject matter provides a physiologically relevant tissue or component thereof produced according to a presently disclosed method of growing a physiologically relevant tissue from at least one cell.

In some aspects, the presently disclosed subject matter provides a method of treating a subject in need thereof, the method comprising implanting into a subject in need thereof a presently disclosed three dimensional hydrogel, a physiologically relevant tissue or component thereof, or the three-dimensional hydrogel together with the physiologically relevant tissue or component thereof. In some embodiments, the subject is in need of the physiologically relevant tissue or component thereof.

In some aspects, the presently disclosed subject matter provides a method of screening for a candidate agent that modulates a hormonal response of a hormone-responsive physiologically relevant tissue or component thereof, the method comprising: (a) contacting a hormone-responsive physiologically relevant tissue or component thereof cultured in a presently disclosed three-dimensional hydrogel with a test agent; and (b) assessing a hormonal-response of the hormone-responsive physiologically relevant tissue or component thereof in the presence of the test agent as compared to the hormonal-response of a control hormone-responsive physiologically relevant tissue or component thereof not contacted with the test agent, wherein a change in the hormonal response of the hormone-responsive physiologically relevant tissue or component thereof in the presence of the test agent indicates that the test agent is a candidate agent that modulates the hormonal response of the hormone-responsive physiologically relevant tissue or component thereof.

In some embodiments of the presently disclosed solution, kit, hydrogel, physiologically relevant tissue, or method, the aqueous medium comprises water. In some embodiments of the presently disclosed solution, kit, hydrogel, physiologically relevant tissue, or method, the collagen is present at a concentration of between 0.5 mg/ml and 4.0 mg/ml. In some embodiments of the presently disclosed solution, kit, hydrogel, physiologically relevant tissue, or method, the fibronectin is present at a concentration of between 1 μg/mL and 50 μg/mL. In some embodiments of the presently disclosed solution, kit, hydrogel, physiologically relevant tissue, or method, the laminin is present at a concentration of between 20 μg/ml and 60 μg/ml. In some embodiments of the presently disclosed solution, kit, hydrogel, physiologically relevant tissue, or method, the laminin comprises laminin isolated from Engelbreth-Holm-Swarm sarcoma cells.

In some embodiments of the presently disclosed solution, kit, hydrogel, physiologically relevant tissue, or method, the hyaluronan is present at a concentration of between 1 μg/mL and 50 μg/mL. In some embodiments of the presently disclosed solution, kit, hydrogel, physiologically relevant tissue, or method, the hyaluronan has a molecular weight ranging from 25 kDA to 1000 kDa. In some embodiments of the presently disclosed solution, kit, hydrogel, physiologically relevant tissue, or method, the hyaluronan comprising a low molecular weight hyaluronic acid and a high molecular weight hyaluronic acid. In some embodiments of the presently disclosed solution, kit, hydrogel, physiologically relevant tissue, or method, the low molecular weight hyaluronan comprises a molecular weight of 150 kDa, and wherein the high molecular weight hyaluronan comprises a molecular weight of 500 kDa.

In some embodiments of the presently disclosed solution, kit, hydrogel, physiologically relevant tissue, or method, the at least one agent that promotes growth of a physiologically relevant tissue is selected from the group consisting of a cytokine, a growth factor, a morphogen, and a steroid hormone.

In some embodiments of the presently disclosed solution, kit, hydrogel, physiologically relevant tissue, or method, the physiologically relevant tissue comprises epithelium. In some embodiments of the presently disclosed solution, kit, hydrogel, physiologically relevant tissue, or method, the physiologically relevant tissue comprises ductal or glandular epithelium. In some embodiments of the presently disclosed solution, kit, hydrogel, physiologically relevant tissue, or method, the physiologically relevant tissue comprises ductal or glandular epithelium tissue selected from the group consisting of colon, gall bladder, intestine, kidney, liver, lung, mammary, pancreas, prostate, and stomach. In some embodiments of the presently disclosed solution, kit, hydrogel, physiologically relevant tissue, or method, the physiologically relevant tissue comprises non-epithelial tissue, e.g., nervous system tissue. In some embodiments of the presently disclosed solution, kit, hydrogel, physiologically relevant tissue, or method, the physiologically relevant tissue comprises mammalian tissue. In some embodiments of the presently disclosed solution, kit, hydrogel, physiologically relevant tissue, or method, the physiologically relevant tissue comprises human or mouse tissue. In some embodiments of the presently disclosed solution, kit, hydrogel, physiologically relevant tissue, or method, the physiologically relevant tissue comprises tissue derived from vertebrate or non-vertebrate animals. In some embodiments of the presently disclosed solution, kit, hydrogel, physiologically relevant tissue, or method, the physiologically relevant tissue comprises a tumor. In some embodiments of the presently disclosed solution, kit, hydrogel, physiologically relevant tissue, or method, the physiologically relevant tissue comprises at least one cell having a mutation in an oncogene or a tumor suppressor gene.

In some embodiments of the presently disclosed solution, kit, hydrogel, physiologically relevant tissue, or method, the at least one cell comprises a single cell. In some embodiments of the presently disclosed solution, kit, hydrogel, physiologically relevant tissue, or method, the at least one cell comprises a single cell selected from the group consisting of a stem cell, a primary cell, a transdifferentiated cell, a dedifferentiated cell, a reprogrammed cell, a multipotent cell, a pluripotent cell. In some embodiments of the presently disclosed solution, kit, hydrogel, physiologically relevant tissue, or method, the at least one cell comprises a single cell selected from the group consisting of a colon cell, a gall bladder cell, an intestine cell, a kidney cell, a liver cell, a lung cell, a mammary cell, an ovarian cell, a cervical cell, a pancreatic cell, and a prostate cell, and a stomach cell. In some embodiments of the presently disclosed solution, kit, hydrogel, physiologically relevant tissue, or method, the at least one cell comprises a single cell selected from the group consisting of a neural crest cell or neural crest derived cell. In some embodiments of the presently disclosed solution, kit, hydrogel, physiologically relevant tissue, or method, the at least one cell comprises a melanocyte. In some embodiments of the presently disclosed solution, kit, hydrogel, physiologically relevant tissue, or method, the at least one cell comprises a single cell selected from the group consisting of a neural cell or glial cell. In some embodiments of the presently disclosed solution, kit, hydrogel, physiologically relevant tissue, or method, the at least one cell comprises a cell line, at least one cluster of cells, or at least one tissue fragment.

In some embodiments of the presently disclosed solution, kit, hydrogel, physiologically relevant tissue, or method, the at least one cluster of cells comprises a cluster of between about 2 and 50 cells, 50 and 100 cells, 100 and 1000 cells, 1000 and 10,000 cells, or 10,000 and 100 million cells. In some embodiments of the presently disclosed solution, kit, hydrogel, physiologically relevant tissue, or method, the at least one cluster of cells comprises a fluorescent protein. In some embodiments of the presently disclosed solution, kit, hydrogel, physiologically relevant tissue, or method, the at least one cluster of cells is depleted for stromal cells. In some embodiments of the presently disclosed solution, kit, hydrogel, physiologically relevant tissue, or method, the at least one cluster of cells is depleted for fibroblasts. In some embodiments of the presently disclosed solution, kit, hydrogel, physiologically relevant tissue, or method, the at least one cluster of cells comprises epithelial cells. In some embodiments of the presently disclosed solution, kit, hydrogel, physiologically relevant tissue, or method, the epithelial cells are not immortalized by transduction with viral oncogenes. In some embodiments of the presently disclosed solution, kit, hydrogel, physiologically relevant tissue, or method, the epithelial cells comprise mammary epithelial cells. In some embodiments of the presently disclosed solution, kit, hydrogel, physiologically relevant tissue, or method, the epithelial cells comprise a disorganized cluster of mammary epithelial cells comprising intermixed CK14+ basal and CK8/18+ luminal cells. In some embodiments of the presently disclosed solution, kit, hydrogel, physiologically relevant tissue, or method, the epithelial cells comprise a disorganized cluster of mammary epithelial cells comprising intermixed CK14+ basal and CK8/18+ luminal cells.

In some embodiments of the presently disclosed solution, kit, hydrogel, physiologically relevant tissue, or method, the at least one agent that promotes growth of a physiologically relevant tissue is epidermal growth factor (EGF) or a functional variant thereof. In some embodiments of the presently disclosed solution, kit, hydrogel, physiologically relevant tissue, or method, the EGF or functional variant thereof is present at a concentration of between 1 ng/mL and 100 ng/mL.

In some embodiments of the presently disclosed solution, kit, hydrogel, physiologically relevant tissue, or method, the at least one agent is insulin or a functional variant, insulin receptor agonist, or mimetic thereof. In some embodiments of the presently disclosed solution, kit, hydrogel, physiologically relevant tissue, or method, the insulin or a functional variant, insulin receptor agonist, or mimetic thereof is present at a concentration of between 1 μg/mL and 100 μg/mL.

In some embodiments of the presently disclosed solution, kit, hydrogel, physiologically relevant tissue, or method, the at least one agent is hydrocortisone or analog or derivative thereof. In some embodiments of the presently disclosed solution, kit, hydrogel, physiologically relevant tissue, or method, the hydrocortisone or analog or derivative thereof is present at a concentration of between 50 ng/mL and 5 μg/mL.

In some embodiments of the presently disclosed solution, kit, hydrogel, physiologically relevant tissue, or method, the mammary epithelial cells are obtained from a subject. In some embodiments of the presently disclosed solution, kit, hydrogel, physiologically relevant tissue, or method, the subject is selected from the group consisting of: (i) a subject who underwent, or is about to undergo, a breast reduction mammoplasty; (ii) a subject who underwent, or is about to undergo, a breast reconstruction or breast augmentation surgery; (iii) a subject has, or is suspected of having, breast cancer; (iv) a subject who has been prescribed, or is taking, an anti-lactogenic medication; (v) a subject for which breastfeeding is contraindicated; (vi) a subject who has, or is suspected of having, lactation failure; and (vii) a subject who has, or is suspected of having, breast hypoplasia, atypical ductal hyperplasia, papillomas, fistulas, inflammation, or other pathological breast conditions. In some embodiments of the presently disclosed solution, kit, hydrogel, physiologically relevant tissue, or method, the breast cancer is selected from the group consisting of ER-positive breast cancer, triple-negative breast cancer, Her2-positive breast cancer, and luminal breast cancer (hormone receptor-positive and -negative). In some embodiments of the presently disclosed solution, kit, hydrogel, physiologically relevant tissue, or method, the subject is a human. In some embodiments of the presently disclosed solution, kit, hydrogel, physiologically relevant tissue, or method, the subject is female. In some embodiments, the subject is male.

In some embodiments of the presently disclosed solution, kit, hydrogel, physiologically relevant tissue, or method, the at least one cell is cultured in the three-dimensional hydrogel in a defined culture medium. In some embodiments of the presently disclosed solution, kit, hydrogel, physiologically relevant tissue, or method, the at least one cell is cultured in the three-dimensional hydrogel in a culture medium that is substantially free of serum. In some embodiments of the presently disclosed solution, kit, hydrogel, physiologically relevant tissue, or method, the at least one cells is cultured in the three-dimensional hydrogel in a culture medium that is free of ROCK inhibitor and forskolin. In some embodiments of the presently disclosed solution, kit, hydrogel, physiologically relevant tissue, or method, the at least one cell is cultured in the three-dimensional hydrogel in a culture medium that comprises at least one agent that stimulates development of mammary tissue in vivo.

In some embodiments of the presently disclosed solution, kit, hydrogel, physiologically relevant tissue, or method, the at least one agent is selected from the group consisting of a steroid hormone, a pituitary hormone, a lactogenic hormone, and derivatives and combinations thereof. In some embodiments of the presently disclosed solution, kit, hydrogel, physiologically relevant tissue, or method, the steroid hormone is selected from the group consisting of estrogen and progesterone. In some embodiments of the presently disclosed solution, kit, hydrogel, physiologically relevant tissue, or method, the estrogen is present at a concentration of between 1 ng/mL and 100 ng/mL. In some embodiments of the presently disclosed solution, kit, hydrogel, physiologically relevant tissue, or method, the progesterone is present at a concentration of between 1 ng/mL and 100 ng/mL. In some embodiments of the presently disclosed solution, kit, hydrogel, physiologically relevant tissue, or method, the pituitary hormone comprises a hormone or growth factor present in a pituitary extract. In some embodiments of the presently disclosed solution, kit, hydrogel, physiologically relevant tissue, or method, the hormone or growth factor present in the pituitary extract is selected from the group consisting of growth hormone, fibroblast growth factor, prolactin and follicle stimulating hormone. In some embodiments of the presently disclosed solution, kit, hydrogel, physiologically relevant tissue, or method, the lactogenic hormone is prolactin.

In some embodiments of the presently disclosed solution, kit, hydrogel, physiologically relevant tissue, or method, when at least one mammary epithelial cell, or at least one cluster of mammary epithelial cells, is cultured in the three-dimensional hydrogel, the at least one mammary epithelial cell, or at least one cluster of mammary epithelial cells, grows into physiologically relevant mammary tissue in the three-dimensional hydrogel. In some embodiments of the presently disclosed solution, kit, hydrogel, physiologically relevant tissue, or method, during growth of the physiologically relevant mammary tissue, the cultured cells and/or growing physiologically relevant mammary tissue exhibits at least one of the following features: i) ductal initiation and/or ductal elongation; ii) a tip at a leading edge of at least one elongating duct, wherein the tip comprises one or two leader cells polarized in the direction of ductal elongation; iii) leader cells expressing basal cytokeratins, staining positively for filamentous actin, and co-expressing SLUG and SOX9; iv) organization into expanding tissues comprising an outer CK14+ basal layer and interior CK8/18+ luminal cells; v) lobule interiors expressing luminal lineage marker GATA3, and luminal differentiation marker MUC1; vi) cavitation of lobule interiors; vii) secondary and tertiary ductal branching selected from the group consisting of bifurcated elongated ducts and side-branches sprouted from primary ducts; viii) lipid droplets; ix) hormone-responsiveness; x) terminal ductal-lobular units (TDLUs), wherein at least a portion of the cells comprising the TDLUs are SLUG+/SOX9+ mammary stem cells; xi) TDLUs comprising layers of between 5 and 8 cells; and xii) expression of hormone receptors selected from the group consisting of estrogen receptors, progesterone receptors, glucocorticoid receptors, and androgen receptors. In some embodiments of the presently disclosed solution, kit, hydrogel, physiologically relevant tissue, or method, when at least one mammary epithelial cell, or at least one cluster of mammary epithelial cells, is cultured in the three-dimensional hydrogel, the cultured cell or cells exhibit increased ductal, lobular and ductal-lobular growth compared to mammary epithelial cells cultured in three-dimensional basement membrane scaffolds or three-dimensional collagen scaffolds. In some embodiments of the presently disclosed solution, kit, hydrogel, physiologically relevant tissue, or method, the physiologically relevant mammary tissue grown in the three-dimensional hydrogel is viable in the three-dimensional hydrogel for at least six weeks. In some embodiments of the presently disclosed solution, kit, hydrogel, physiologically relevant tissue, or method, the physiologically relevant mammary tissue grown in the three-dimensional hydrogel exhibits ductal-lobular morphologies observed in human breast tissue in vivo. In some embodiments of the presently disclosed solution, kit, hydrogel, physiologically relevant tissue, or method, the physiologically relevant mammary tissue grown in the three-dimensional hydrogel secretes milk. In some embodiments of the presently disclosed solution, kit, hydrogel, physiologically relevant tissue, or method, the milk is human milk.

In some aspects, the presently disclosed subject matter provides a method for producing hormone-responsive, milk-producing mammary tissue, the method comprising culturing at least one mammary epithelial cell or at least one cluster of mammary epithelial cells in a presently disclosed three-dimensional hydrogel in the presence of at least one agent that stimulates development of mammary tissue in vivo for a sufficient amount of time to produce hormone-responsive, milk-producing mammary tissue.

In some aspects, the presently disclosed subject matter provides a method for producing milk, the method comprising culturing the hormone-responsive, milk-producing mammary tissue produced according to the method of producing hormone-responsive, milk producing mammary tissue for a sufficient amount of time to produce an amount of milk.

In some aspects, a method of screening for a candidate agent that modulates milk production, the method comprising: (a) culturing the hormone-responsive, milk-producing mammary tissue produced according to the method of producing hormone-responsive, milk producing mammary tissue in the presence of a test agent; and (b) measuring an amount of milk produced by the hormone-responsive, milk-producing mammary tissue in the culture in the presence of the test agent as compared to a control amount of milk production, wherein a change in amount of milk produced by the hormone-responsive, milk-producing mammary tissue in the culture in the presence of the test agent as compared to the control amount of milk production indicates that the test agent is a candidate agent that modulates milk production.

In some embodiments, the test agent is candidate agent that decreases milk production and a decrease in the amount of milk produced by the hormone-responsive, milk-producing mammary tissue indicates that the test agent is a candidate agent that inhibits milk production. In some embodiments, the test agent is a candidate agent that increases milk production and an increase in the amount of milk produced by the hormone-responsive, milk-producing mammary tissue indicates that the test agent is a candidate agent that increases milk production. In some embodiments, the amount of milk produced is determined by quantifying milk lipids, milk carbohydrates, and/or milk proteins. In some embodiments, the lipids are quantified using dyes and stains. In some embodiments, the dyes and stains are selected from the group consisting of oil red o, nile red, and 1,6-Diphenyl-1,3,5-hexatriene. In some embodiments, the lipids are quantified using haematoxylin and eosin staining. In some embodiments, the milk proteins are quantified using antibodies. In some embodiments, the milk proteins are quantified using an analytical technique selected from the group consisting of mass spectrometry, Western blot, enzyme-linked immunosorbent assay (ELISA), immunohistochemistry, and immunofluorescence. In some embodiments, the milk carbohydrates are quantified using colorimetric, mass spectrometric, or fluorescent based assays. In some embodiments, the amount of milk produced is quantified microscopically based on opacity of the cultures. In some embodiments, the candidate agent is a fractionated portion of a mixture to identify the active ingredient in the mixture that modulates milk production. In some embodiments, the mixture comprises an herbal supplement. In some embodiments, the herbal supplement comprises fenugreek.

In some aspects, the presently disclosed subject matter provides a method for treating a subject in need of treatment thereof, the method comprising administering to the subject in in need thereof a candidate agent identified according to a presently disclosed screening method.

In some aspects, the presently disclosed subject matter provides a method for treating a subject in need thereof, the method comprising: (a) obtaining at least one mammary epithelial cell or at least one cluster of mammary epithelial cells; (b) culturing the at least one mammary epithelial cell or the at least one cluster of mammary epithelial cells in a presently disclosed three-dimensional hydrogel optionally in the presence of at least one agent that stimulates development of mammary tissue in vivo for a sufficient amount of time for outgrowth of the at least one mammary epithelial cell or the at least one cluster of mammary epithelial cells in the three-dimensional hydrogel to occur; and (c) implanting the three-dimensional hydrogel into the subject.

In some embodiments, the at least one mammary epithelial cell or at least one cluster of mammary epithelial cells are selected from the group consisting of allogeneic cells and autologous cells.

In some aspects, the presently disclosed subject matter provides a method for screening a candidate agent that modulates milk production comprising: (a) administering a candidate agent that modulates milk production to a subject (e.g., a subject with a presently disclosed three-dimensional hydrogel implanted into it (e.g., a three-dimensional hydrogel comprising at least one cell cultured in it or a physiologically relevant tissue grown in it); and (b) measuring milk production in the subject, wherein a change in milk production in said subject as compared to a control identifies said agent as a candidate agent that modulates milk production.

In some embodiments, prior to administering the candidate agent to said subject the mammary epithelial cells are allowed to grow for a sufficient amount of time for the mammary tissue to mature and hallow. In some embodiments, said measuring milk production comprises measuring a volume of milk produced prior to and after administering the candidate agent, and wherein said control is the measured volume of milk produced prior to administering the candidate agent.

In some aspects, the presently disclosed subject matter provides a method of evaluating the effect of an agent on a biological condition of cells, the method comprising: (a) providing a presently disclosed three-dimensional hydrogel; (b) culturing at least one cell or at least one cluster of cells in the three-dimensional hydrogel for a period of time sufficient for the at least one cell or at least one cluster of cells to expand in the three-dimensional hydrogel; and (c) exposing the expanding cells in the three-dimensional hydrogel to an agent; and (d) evaluating the effect of the agent on the biological condition of the cells. In some aspects, such methods may comprise toxicology assays.

In some aspects, the presently disclosed subject matter provides a method of evaluating the effect of an agent on a biological condition of a physiologically relevant tissue, the method comprising: (a) providing a presently disclosed three-dimensional hydrogel; (b) culturing at least one cell or at least one cluster of cells in the three-dimensional hydrogel for a period of time sufficient for a physiologically relevant tissue to grow in the three-dimensional hydrogel; and (c) exposing the physiologically relevant tissue growing in the three-dimensional hydrogel to an agent; and (d) evaluating the effect of the test agent on the biological condition of the physiologically relevant tissue. In some aspects, such methods may comprise toxicology assays.

In some embodiments, the physiologically relevant tissue comprises epithelium tissue selected from the group consisting of colon, gall bladder, kidney, liver, lung, mammary, pancreas, and prostate. In some embodiments, the at least one cell or at least one cluster of cells is selected from the group consisting of a single cell, a cell line, a stem cell, a primary cell, a transdifferentiated cell, a dedifferentiated cell, a reprogrammed cell, a multipotent cell, and a pluripotent cell. In some embodiments, the at least one cell or at least one cluster of cells is selected from the group consisting of a colon cell, a gall bladder cell, a kidney cell, a liver cell, a lung cell, a mammary cell, an ovarian cell, a cervical cell, a pancreatic cell, and a prostate cell.

In some embodiments, the physiologically relevant tissue comprises neural crest tissue or neural crest derived tissue. In some embodiments the neural crest derived tissue comprises melanocytes. In some embodiments the neural crest derived tissue comprises craniofacial cartilage, craniofacial bone, smooth muscle, dorsal root ganglia, sympathetic ganglia, adrenal medulla, enteric nervous system, and parasympathetic ganglia. In some embodiments the at least one cell comprises a melanocyte. In some embodiments the at least one cell or cluster of cells comprises a ganglion-derived cell or ganglion-derived cell cluster, which for purposes of the present disclosure refers to a cell or cluster of cells that is isolated from a ganglion or is descended from a cell or cluster of cells that was isolated from a ganglion. For purposes of the present disclosure, unless otherwise indicated, a “ganglion” refers to a nerve cell cluster or a group of nerve cell bodies located in the nervous system, e.g., the autonomic nervous system (dorsal root ganglia, sympathetic ganglia, parasympathetic ganglia), enteric nervous system, or central nervous system (e.g., basal ganglia). In some embodiments the at least one cell comprises a neural cell. A neural cell may be a neural progenitor cell or a neuron. In some embodiments the neuron is a peripheral nervous system neuron. In some embodiments the neuron is a sensory neuron. In some embodiments the neuron is a motor neuron. In some embodiments the neuron is a central nervous system neuron. In some embodiments the neuron is a dopaminergic neuron, glutamatergic neuron, GABAergic neuron, cholinergic neuron, or serotonergic neuron. In some embodiments the at least one cell comprises a glial cell. In some embodiments the glial cell is an astrocyte or oligodendrocyte. In some embodiments the glial cell is a Schwann cell. In some embodiments the glial cell is a myelin-producing cell.

In some embodiments, the at least one cell or at least one cluster of cells comprise cancerous cells, or cells having at least one mutation in an oncogene or a tumor suppressor. In some embodiments, the at least one cell or at least one cluster of cells is obtained from a subject. In some embodiments, the subject is a normal healthy subject. In some embodiments, the subject is at risk of developing a disease, condition, or disorder. In some embodiments, the subject is suffering from a disease, condition, or disorder. In some embodiments, the at least one cell or at least one cluster of cells comprise neural cells derived from a subject suffering from a neurodegenerative disease.

In some embodiments, evaluating the effect of the agent on the biological condition comprises imaging cells in the three-dimensional hydrogel to determine how the agent affects a phenotype of the cells. In some embodiments, evaluating the effect of the agent on the biological condition identifies at least one of a change in growth rate, cell number, cell shape, viability, function, and morphology of the cells. In some embodiments, evaluating the effect of the agent on the biological condition comprises conducting an omic analysis on the cells selected from the group consisting of genomic analysis, metabolomic analysis, proteomic analysis, and a transcriptomic analysis. In some embodiments, evaluating the effect of the agent on the biological condition comprises conducting an epigenetic analysis on the cells.

In some embodiments in which the cells comprise neurons, evaluating the effect of the agent on the biological condition comprises detecting at least one of: neural activity (e.g., action potential, depolarization, ion flux), expression of neural markers (e.g., neurofilament proteins such as NF200; NeuN; channel proteins), neurite outgrowth, axon and/or dendrite formation or growth, neurotransmitter production, activity of an enzyme involved in neurotransmitter synthesis or breakdown, synapse formation, or a change in any of the foregoing. In some embodiments in which the cells comprise neurons and glial cells, evaluating the effect of the agent on the biological condition comprises detecting myelin production, myelination, or a change in myelin production or myelination. One of ordinary skill in the art is aware of suitable assays for detecting any of the foregoing properties or parameters. For example, myelin may be detected using a suitable stain such as Oil Red O or FluoroMyelin™ Red (ThermoFisher). Neurite, dendrite, and/or axon growth may be detected microscopically, e.g., using suitable reagents to visualize the processes. In some embodiments, the agent is a chemical compound or a biological material. In some embodiments, the agent is electromagnetic radiation, particle radiation, a non-ambient temperature, a non-ambient pressure, acoustic energy, a mechanical force, an electrical field, a magnetic field, and combinations thereof. In some embodiments, the agent is a candidate agent selected from the group consisting of a candidate allergenic agent, a candidate biologic agent, a candidate carcinogenic agent, a candidate estrogenic agent, a candidate immunogenic agent, a candidate lactogenic agent, a candidate mutagenic agent, a candidate nerve agent, a candidate pathogenic agent, a candidate pesticide agent, a candidate radioactive agent, a candidate teratogenic agent, a candidate toxicant agent, and candidate vesicant agent. In some embodiments, the agent is an industrial chemical. In some embodiments, the agent is bisphenol A (BPA) or another industrial chemical suspected of being harmful to human health. In some embodiments, the agent is a candidate therapeutic agent. In some embodiments, the candidate therapeutic agent is a candidate chemotherapeutic agent. In some embodiments, the candidate therapeutic agent is a candidate neuromodulatory agent.

In some embodiments, the biological condition is normal unperturbed functioning of a cell, organ or tissue and the agent causes one or more of the cells to become abnormal. In some embodiments, the biological condition is a disease or perturbed functioning of a cell, organ or tissue and the agent causes one or more of the cells to become normal. In some embodiments, the biological condition is selected from the group consisting of cancer, diabetes (e.g., prediabetes, Type I diabetes, Type II diabetes, metabolic syndrome), a neurodegenerative disease, a cardiovascular disease, or an auto-immune disease. In some embodiments, the biological condition is a cancer, and wherein the cells comprise cancerous epithelial cells from the same tissue or organ. In some embodiments, the cancer is selected from the group consisting of colon cancer, gall bladder cancer, kidney cancer, liver cancer, lung cancer, mammary cancer, ovarian cancer, cervical cancer, pancreatic cancer, and prostate cancer. In some embodiments, the cancer is a skin cancer. In some embodiments the cancer is a melanoma. In some embodiments the cancer is a cancer of the peripheral nervous system. In some embodiments the cancer is a cancer of the central nervous system. In some embodiments the tumor of the central or peripheral nervous system is a glioma, ganglioglioma, or neuroblastoma. In some embodiments a glioma is an astrocytoma. In some embodiments a glioma is glioblastoma multiforme (a malignant astrocytoma).

In some aspects, the presently disclosed subject matter comprises a method of screening for a candidate chemotherapeutic agent, the method comprising: (a) culturing at least one cancer cell in a presently disclosed three-dimensional hydrogel for a sufficient amount of time for growth of the at least one cancer cell in the three-dimensional hydrogel to occur; (b) exposing the at least one cancer cell in the three-dimensional hydrogel to at least one test agent; and (c) measuring growth of the at least one cancer cell in the three-dimensional hydrogel in the presence of the test agent, wherein a decrease in growth of the at least one cancer cell in the presence of the test agent as compared to a control identifies the agent as a candidate chemotherapeutic agent.

In some embodiments, the at least one cancer cell is cultured for a sufficient amount of time to expand the at least one cancer in the culture by at least 2-fold. In some embodiments, the at least one cancer cell is cultured for a sufficient amount of time to produce tumor spheroids in the three-dimensional hydrogel. In some embodiments, the at least one cancer cell is cultured for a sufficient amount of time for the at least one cancer cell to exhibit cell invasion in the three-dimensional hydrogel. In some embodiments, the at least one cancer cell is cultured for a period of between about one week and about two weeks. In some embodiments, the cancer cells are cultured in hypoxic oxygen conditions. In some embodiments, the cancer cells are cultured in hypoxic oxygen conditions comprising between 0.1% and 1.0% oxygen. In some embodiments, the at least one cancer cell is obtained by dissociating tumor tissue obtained from a subject into a single cell. In some embodiments, the at least one cancer cell is obtained from an in situ or pre-malignant lesion of the subject. In some embodiments, the subject has breast cancer. In some embodiments, the breast cancer is selected from the group consisting of ER-positive breast cancer, triple-negative breast cancer, Her2-positive breast cancer, and luminal breast cancer (hormone receptor-positive and -negative). In some embodiments, the subject's breast tumor expresses at least one hormone receptor. In some embodiments, the at least one cancer cell retains expression of the at least one hormone receptor in culture in the three-dimensional hydrogel. In some embodiments, the at least one hormone receptor is selected from the group consisting of an epidermal growth factor receptor (EGFR), estrogen receptor, HER2 receptor, a MET receptor, a progesterone receptor, a glucocorticoid receptor, and an androgen receptor. In some embodiments, the subject has melanoma.

In some embodiments, measuring growth of the at least one cancer cell comprises measuring cell proliferation or measuring cell viability of the at least one cancer cell in the three-dimensional hydrogel. In some embodiments, measuring growth of the at least one cancer cell comprises counting surviving cancer cells using microscopy. In some embodiments, the method further comprises: (i) quantifying said surviving cancer cells using dyes and stains that identify living cells; (ii) quantifying said surviving cancer cells using a plate-reader in combination with dyes and stains that identify living cells; (iii) quantifying said surviving cancer cells using a plate-reader in combination with a reagent that emits a luminescent signal, a fluorescent signal, or colorimetric signal when contacted with living cells; or (iv) quantifying said surviving cancer cells by barcoding via infection with a pool of retroviruses or lentiviruses, and sequencing DNA to determine the number of said surviving cancer cells. In some embodiments, measuring growth of the at least one cancer cell is performed after three days of exposing the at least one cancer cell in the three-dimensional hydrogel to the candidate chemotherapeutic agent.

In some embodiments, the candidate chemotherapeutic agent is selected from the group consisting of a small organic compound, RNA, DNA, peptide, and an antibody. In some embodiments, the candidate chemotherapeutic agent is selected from the group consisting of RNAi, shRNA, and a genomic editing system. In some embodiments, the genomic editing system is selected from the group consisting of a CRISPR-Cas system, a meganuclease, a zinc finger nuclease, and a transcription activator-like effector-based nuclease (TALEN).

In some embodiments, the at least one cancer cell is exposed to multiple test agents in the three-dimensional hydrogel. In some embodiments, method further comprises selecting a combination of agents which when used together results in the greatest decrease in growth or a selected decrease in growth of the cancer cells in the three-dimensional hydrogel.

In some aspects, the presently disclosed subject matter provides a method for personalized treatment of a cancer in a patient in need thereof, the method comprising administering to the patient the combination of agents selected in in accordance with a presently disclosed method (e.g., screening and/or evaluating an effect of an agent on a biological condition of a cell or physiologically relevant tissue. In some embodiments, the method further comprises monitoring growth or survival of cancerous cells in the patient.

In some aspects, the presently disclosed subject matter provides an immunocompromised animal comprising a presently disclosed three-dimensional hydrogel, or a hydrogel precursor solution thereof, implanted into it. In some embodiments, the animal comprises a rodent. In some embodiments, the rodent comprises a mouse. In some embodiments, the mouse comprises an immunocompromised strain selected from the group consisting of nude, Rag, NOD/SCID or gamma2-null. In some embodiments, the three-dimensional hydrogel is implanted into the mammary gland, under the kidney capsule, or subcutaneously into the animal. In some embodiments, the three-dimensional hydrogel comprises a patient tumor xenograft comprising at least one cell obtained from a patient suffering from a disease, wherein the at least one cell is dissociated from a patient's diseased tissue is cultured in the three-dimensional hydrogel. In some embodiments, the at least one cell comprises at least one cancer cell. In some embodiments, the cancer cells are cultured in the three-dimensional hydrogel for a period of time between about 1 minute and about 1 month before implanting the three-dimensional hydrogel into the animal.

In some aspects, the presently disclosed subject matter provides a method of screening for a personalized candidate chemotherapeutic regimen for a patient in need thereof, the method comprising: (a) administering a combination of candidate chemotherapeutic agents to the immunocompromised animal; (b) measuring growth and survival of cancer cells in the animal; and (c) selecting the combination of candidate chemotherapeutic agents resulting in the greatest decrease in growth and survival of cancer cells or a selected decrease in growth and survival of cancer cells in the animal as a personalized candidate chemotherapeutic regimen for the patient in need thereof.

In some aspects, three dimensional culture models described herein may be used for drug development and/or toxicology assays.

The practice of the presently disclosed subject matter will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning. A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange 10^{th} ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL: http://www.ncbi.nlm.nih.gov/omim/ and in Online Mendelian Inheritance in Animals (OMIA), a database of genes, inherited disorders and traits in animal species (other than human and mouse), at http://omia.angis.org.au/contact.shtml. Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1A, FIG. 1B, FIG. 1C, FIG. 1D, FIG. 1E, and FIG. 1F, show that the presently disclosed three-dimensional hydrogels enable self-organization and growth of human breast tissue. FIG. 1A is a schematic representation of hydrogel assembly. FIG. 1B depicts representative bright-field images of tissue growth after ten days in Matrigel, polymerized collagen or the presently disclosed three-dimensional hydrogels (e.g., in the absence of BPE), alongside carmine stained mammary tissue sections from independent reduction mammoplasty patients. FIG. 1C shows quantification of the frequency of a tissue fragment producing a tissue outgrowth (Freq. Formation) and the frequency of a tissue maturing from a patient, as determined by the formation of TDLUs (Freq. Maturation). FIG. 1D shows treatment with estrogen (10 ng/mL) and progesterone (500 ng/mL) (+EP) for three weeks caused ducts and lobules to hollow (red arrowheads). FIG. 1E depicts schematic and representative images showing the effect of pituitary hormones (0.4% bovine pituitary extract [BPE]) and 1 μg/mL recombinant human prolactin administration on the development of tissue structures grown in the presently disclosed three-dimensional hydrogels. BPE was added at seeding (D0) and prolactin at two weeks (N=4, 7 and 14, 1 resp). Bottom left images show representative architecture prior to the administration of prolactin. FIG. 1F shows quantification of average lobular volume at day 21. Error bars represent SEM. Scale bars are 200 μm. * p<0.01

FIG. 2A, FIG. 2B, FIG. 2C, FIG. 2D, and FIG. 2E show human breast tissue as it undergoes morphogenesis and differentiation in the presently disclosed three-dimensional hydrogels. FIG. 2A shows bright-field images (top) and a schematic representation (bottom) of the dramatic expansion and maturation of tissue structures over the course of twelve days. Scale bar is 200 μm. FIG. 2B shows quantification of the number of ducts per tissue structure, lobules per tissue structure, and cross-sectional area of tissue structures during a 12 day timecourse. N=9 tissue structures. FIG. 2C shows IF of luminal (CK8/18) and myoepithelial (CK14) markers reveal that at seeding (left), tissue fragments are disorganized, but self-organize into a two-layered structures within seven days (center). By 11 days after seeding (right), outgrowths have matured and the CK8/18⁺ luminal layer fully lines the interior. Inset scale bar is 50 μm. All other scale bars are 200 μm. FIG. 2D shows IHC of tissue structures after 21 days of culture revealing a hollowing lumen surrounded by cells positive for MUC1 and GATA3. Representative image from one patient. FIG. 2E shows clonal tracking using RGB lentivirus demonstrating the dynamic nature of cells within the tissue structures. Image depicts RGB signal superimposed on the profile of the tissue structures, captured using bright-field. Error bars represent SEM.

FIG. 3A, FIG. 3B, FIG. 3C, FIG. 3D, FIG. 3E, and FIG. 3F, show side branching mediated by SLUG⁺/SOX9⁺ cell expansion. FIG. 3A shows IF of putative MaSC markers SLUG and SOX9 reveals that tissue outgrowths (day 10) are enriched for dual positive cells. Scale bar is 200 μm. (Right): high magnification images reveal that new side-branches are enriched for SLUG⁺/SOX9⁺ cells. Scale bar is 200 μm. FIG. 3B depicts (Top): IF imaging reveals that SLUG⁺/SOX9⁺ cells localize to the leading edge of elongating outgrowths. Arrows indicate direction of growth. Scale bars are 50 μm. FIG. 3C shows 3D-printed model of tissue structures from FIG. 3A (bottom). The model helped to show leader cells located at the leading edge of many tissue outgrowths (arrowheads). IF images depict representative outgrowths with long (left), intermediate (top right) and short (bottom right) ductal elongation. FIG. 3D shows quantification of fraction of cells SLUG⁺/SOX9⁺ and duct length reveals a significant anti-correlation, indicating that ductal elongation is concurrent with stem cell differentiation. FIG. 3E shows quantification of fraction of cells expressing SLUG and/or SOX9 in the indicated structure types from the sample depicted in FIG. 3A. Every pairwise comparison was statistically significant (p<0.001). N indicates the number of cells quantified. FIG. 3F depicts BrdU incorporation for 2 hrs shows that proliferating cells in tissue structures (day 10) grown with BPE are found in the cap region, where putative MaSCs are primarily located; patient depicted in FIG. 3A. Scale bar is 200 μm.

FIG. 4A, FIG. 4B, FIG. 4C, FIG. 4D, FIG. 4E, and FIG. 4F shows leader cells drive tissue structure morphogenesis in the presently disclosed three-dimensional hydrogels. FIG. 4A depicts representative images, and quantification, of leader cells that co-express putative MaSC markers SLUG and SOX9. IF shows actin-rich protrusions from leader cells and co-positivity of SLUG and SOX9. Scale bars are 50 μm. FIG. 4B depicts representative images, and quantification, of leader cells expressing myoepithelial marker CK14. Scale bars are 50 μm. FIG. 4C shows bright-field images of TDLUs containing two divergent leader cells (arrowheads). FIG. 4D shows time-lapse analysis revealing the protrusion of leader cells precedes ductal elongation. FIG. 4E is a schematic depiction of FIG. 4D, showing the protrusion of leader cells (red) preceding elongation, indicated by arrows. FIG. 4F is a time-lapse analysis showing a leader cell redirecting the orientation of elongation of a tissue outgrowth. Scale bars are 50 μm.

FIG. 5 is a schematic showing the dissociation of reduction mammoplasty tissue. It is a schematic representation of tissue dissociation and purification of epithelium.

FIG. 6A and FIG. 6B illustrate physical characterization of collagen and the presently disclosed three-dimensional hydrogels. FIG. 6A shows that Young's Modulus was measured at least three times for each of three independent replicates (red, blue, green) for collagen gels and presently disclosed three-dimensional hydrogels. Plotted is the mean and standard deviation for the replicates. FIG. 6B shows the swelling ratio was calculated for collagen gels and presently disclosed three-dimensional hydrogels for four independent replicates. Plotted is the mean and standard deviation. *p<0.05.

FIG. 7A and FIG. 7B show a comparison of various three-dimensional scaffolds seeded with mouse or human mammary tissue. FIG. 7A shows representative bright-field images of human or mouse mammary epithelial tissue fragments grown for 10 days in either Matrigel alone (Matrigel); Matrigel supplemented with fibronectin, laminins, hyaluronans, insulin, epidermal growth factor, and hydrocortisone (Matrigel+ECM); collagen hydrogels (Collagen gel); or the presently disclosed three-dimensional hydrogels comprising collagen, fibronectin, laminin, hyaluronan, insulin, EGF, and hydrocortisone (ECM hydrogel). FIG. 7B shows representative brightfield images of a tissue structure grown in a presently disclosed three-dimensional hydrogel (left), removed from the primary gel using collagenase treatment and fragmented (middle), and producing new outgrowths after being passaged into a secondary three-dimensional hydrogel (right). Scale bars are 200 μm.

FIG. 8 shows single mammary epithelial cells produce heterogeneous structure morphologies in the presently disclosed three-dimensional hydrogels. Representative bright-field and immunofluorescence images of structures formed from single cells after 18 days of growth in the three-dimensional hydrogels. These structures are highly heterogeneous, but generally fall into three classes: ductal structures with narrow ducts and no lobules (top), lobular structures with short and wide ducts (middle), and structures with mixed ductal and lobular architecture (bottom). The majority of structures formed from single cells are either exclusively ductal or exclusively lobular, with only 4.5% of structures scored showing mixed architecture. Immunofluorescence staining for luminal and basal cytokeratins demonstrates that even mixed-architecture tissue structures derived from single-cells do not contain both mature cell types, as CK8/18 staining was never observed. Scale bars are 200 μm.

FIG. 9A and FIG. 9B show tissue structures cultured in the presently disclosed three-dimensional hydrogels respond to hormone treatment. FIG. 9A shows H&E staining of tissue structures treated with either vehicle, or with prolactin (1 μg/mL). Lipid droplets can be seen following prolactin treatment. FIG. 9B shows (Left): confocal maximum intensity projection of a tissue structure grown for 3 weeks in estrogen (10 ng/mL) and progesterone (500 ng/mL), and (Right): serial sections through the tissue structure show hollow ducts and lobules. Distance of the section from the surface of the structure is indicated. Scale bars are 200 μm.

FIG. 10 shows expansion and maturation of tissue structures. Bright-field microscopy demonstrates massive expansion and maturation of tissue structures, with lobule formation initiating after day 5 and maturation of TDLUs by day 12. Note that by day 12 the surrounding three-dimensional hydrogel has been dramatically condensed, leading to reduced visibility. Scale bars are 500 μm.

FIG. 11A and FIG. 11B show that tissue structures self-organize and differentiate. FIG. 11A depicts Immunofluorescence microscopy showing that a myoepithelial layer (CK14, green) completely surrounds the exterior of a tissue structure after 7 days of growth in a presently disclosed three-dimensional hydrogel, while a luminal layer (CK8/18, red) forms in the interior. Note that smaller outgrowths from the central core are exclusively myoepithelial, while larger, more mature outgrowths contain a luminal layer. FIG. 11B shows tissue structures after 14 days of growth in the presently disclosed three-dimensional hydrogels. Scale bars are 200 μm.

FIG. 12A and FIG. 12B show that tissue structures are capable of growing up to 3 mm in diameter. FIG. 12A shows confocal microscopy of actin (phalloidin, red) and nuclear (DAPI, blue) staining of a tissue structure grown for three weeks in a presently disclosed three-dimensional hydrogel. Scale bar is 2 mm. FIG. 12B shows bright-field imaging of tissue structures grown for two weeks (left) and four weeks (right) in the presently disclosed three-dimensional hydrogels. Scale bars are 1 mm.

FIG. 13A and FIG. 13B show that tissue structures perform long-range extra-cellular matrix (ECM) remodeling. FIG. 13A shows bright-field image of tissue structures grown in a presently disclosed three-dimensional hydrogel for four weeks. Note the condensed ECM spanning the distance between the two large tissue structures, in a non-cellular region of the presently disclosed three-dimensional hydrogel. Contrast was increased to better distinguish condensed ECM from uncondensed ECM. Scale bar is 1 cm. FIG. 13B depicts time course bright-field microscopy showing that tissue structures seeded into a presently disclosed three-dimensional hydrogel, at an initial distance of roughly 1 mm apart, align their outgrowths to grow towards one another, indicating long range communication through the three-dimensional hydrogel. By day 12 of growth in the three-dimensional hydrogel (bottom), the three tissue structures have fused together. Scale bars are 0.5 mm.

FIG. 14A shows patient tumor cells expand two-fold over two weeks of growth in the presently disclosed three-dimensional hydrogels. FIG. 14B shows confocal microscopy of patient tumor cells grown in a presently disclosed three-dimensional hydrogel for two weeks and stained the live cell stain, DRAQ5. Example of expanded foci can be seen in the enlarged image.

FIG. 15A illustrates that the tissue structures grown in the presently disclosed three-dimensional hydrogel for four days maintain the expression of ER and PR. Shown are representative IHC images of tissue outgrowths. Black arrows indicate a subset of positive cells. FIG. 15B shows tissue structures grown for two weeks in the presence of vehicle or chemical X (2 uM). The chemical prevented the formation of lobules and resulted in tissue structures only containing ductal outgrowths. FIG. 15C shows a high throughput 3D drug screen revealed drugs that inhibit cancer cell invasiveness. One example of a hit from the screen (Drug 2A11) is shown.

FIG. 16A shows bright field images of tissue outgrowths grown in the presence of estrogen and progesterone for 2 weeks. Black arrows indicate regions of hollowing. FIG. 16B shows bright field image of tissue outgrowths grown with estrogen and progesterone for 2 weeks then with prolactin for an additional week. Arrowheads indicate regions where a dark substance has filled the cavities. FIG. 16C shows oil red 0 staining for lipids on cryosectioned structures treated with EP for 2 weeks and prolactin for 1 week reveals the presence of positive staining in the alveoli as revealed by the presence of red droplets. FIG. 16D shows H&E staining of EP+Prolactin treated organoids reveals the presence of lipid vesicles in the alveoli (indicated by a black arrow).

FIG. 17 shows that treatment of day 14 tissues with estrogen and prolactin induced expression of milk/lactation associated genes (LALBA, BCAS, CD36, SLC5A1) within 7 days.

FIG. 18A shows images of cultures of primary tumor samples derived from melanoma at the time of seeding in 3D hydrogen culture and after 14 days of growth. The 3D gels are condensing and are dark in color (indicating that they are melanin-rich). FIG. 18B shows cell counts at seeding and after 14 days. FIG. 18C shows images of melanoma growths in 3D cultures at 5, 10, and 14 days. Note the dendritic pattern of growth.

FIG. 19 shows growth curves for 6 breast cancer samples expanded in 3D hydrogel culture. Cells were counted at seeding and then upon passaging (up to 21 days). Plotted are total cell numbers at the time of each passage. Panels for MECA2#1 and MECA5 show data for two individual cultures. Panel for MECA7 shows data for three individual cultures.

FIG. 20A shows that morphologies of breast cancer samples grown in 3D culture resemble the descriptions in the pathology report on the cancers from which the samples were obtained. The left panel is an image showing that an invasive carcinoma grew as scattered cells in culture. The right panel is an image showing that an in situ lobular tumor grew as encapsulated clusters. FIG. 20B shows that a breast cancer sample obtained from the same cancer as the sample shown in the right panel of FIG. 20A only produced cells with a fibroblast morphology when cultured in 2D culture Shown is an image at 2 weeks after seeding.

FIG. 21 shows that breast cancer samples cultured in 3D culture exhibit sensitivity to tamoxifen. Shown are cell counts for each dose of drug following 48 hours of treatment. Cells were counted using CellTiter-Glo assay performed on intact gels. FIG. 22A shows immunofluorescence images showing expression of NaV1.7 in newly grown processes in single neurons collected from dissociated murine dorsal root ganglia. FIG. 22B shows immunofluorescence images showing expression of NaV1.7 in newly grown processes in partially dissociated dorsal root ganglia. Both cultures were grown in hydrogel culture for nine days with 40 ng/mL nerve growth factor.

The patent or application file contains at least one drawing executed in color. Copies of this patent or patent application publication with color drawings will be provided by the Office upon request and payment of the necessary fee.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

The presently disclosed subject matter provides hydrogel precursor compositions (e.g., solutions) for forming three-dimensional hydrogels that support growth of physiologically relevant tissue when at least one cell is cultured in the three-dimensional hydrogel, kits comprising the hydrogel precursor composition, three-dimensional hydrogels, methods of forming the three-dimensional hydrogels, methods of growing the physiologically relevant tissue using the three-dimensional hydrogels, physiologically relevant tissue grown in the three-dimensional hydrogels, methods of producing hormone-responsive tissue (e.g., milk-producing mammary tissue and related methods of producing milk), methods of screening for candidate agents useful for modulating hormonal responses (e.g., modulating milk production), methods of screening for candidate therapeutic agents using the physiologically relevant tissue grown in the three-dimensional hydrogels (e.g., personalized cancer treatments), and related methods of treatment (e.g., administering agents identified using the methods herein, transplanting physiologically relevant tissue produced using the methods, etc.).

Three-dimensional (3D) cultures have proven invaluable for expanding human tissues for research or clinical applications. For both applications, 3D cultures are most useful when they (1) support the outgrowth of tissues from primary human cells that have not been immortalized through extensive culture or viral infection, and (2) include defined, physiologically-relevant components. Work described herein reports what is believed to be the first three-dimensional hydrogel exhibiting both of these properties and demonstrates that such three-dimensional hydrogels can stimulate the outgrowth of morphologically complex and hormone-responsive tissue (e.g., morphologically complex and hormone-responsive mammary tissue) from primary human cells.

In one example embodiment, primary human mammary cells from patient reduction mammoplasties were seeded into three-dimensional hydrogels composed of structural protein and carbohydrate components of human breast tissue, and were cultured in serum-free medium containing only defined components. The physical properties of the hydrogels were determined using atomic force microscopy. Tissue development in the hydrogels was monitored using microscopy, and differentiation was gauged both morphologically and by immunostaining for markers of mammary cell types. The tissue outgrowths were also studied by constructing 3D-graphical models from confocal images, which were subsequently printed using a 3D printer. Surprisingly and unexpectedly, when seeded into the three-dimensional hydrogels, primary patient-derived cells self-organized in the absence of stromal cells and grew into complex mammary tissues within as little as 10-14 days. In addition, the expanded tissues responded to mammary hormones, included luminal, basal and stem cell types in the proper orientation, and generated the intricate ductal-lobular morphologies that are observed in the human breast. Ductal branching was initiated by clusters of cells expressing putative mammary stem cell markers, which subsequently localized to the leading edge of tissue outgrowths. Ductal elongation was preceded by leader cells that protruded from the tips of ducts and engaged with the extracellular matrix. The above example illustrates that the presently disclosed three-dimensional hydrogels support the growth of complex tissue from primary patient-derived cells, such as growth of complex mammary tissues exhibiting complex ductal-lobular morphologies observed in human breast tissue.

In another example embodiment, primary human melanoma cells were shown to survive, proliferate, and maintain melanin production in 3D hydrogel culture described herein.

### I. Hydrogel Precursor Composition

Aspects of the presently disclosed subject matter relate to hydrogel precursor compositions (e.g., solutions) for forming three-dimensional hydrogels that support growth of physiologically relevant tissue when at least one cell is cultured in the three-dimensional hydrogel.

Accordingly, in some aspects, the presently disclosed subject matter provides a hydrogel precursor solution for forming a three-dimensional hydrogel that supports growth of a physiologically relevant tissue when at least one cell is cultured in the three-dimensional hydrogel, the hydrogel precursor solution consisting of, consisting essentially of, or comprising: (a) an aqueous medium; (b) at least three hydrogel precursor components dissolved in the aqueous medium to form a hydrogel precursor solution for forming a three-dimensional hydrogel that supports growth of physiologically relevant tissue, wherein the at least three hydrogel precursor components comprise: (i) a first hydrogel precursor component comprising an extracellular matrix protein selected from the group consisting of collagen, fibronectin, and laminin; (ii) a second hydrogel precursor component comprising hyaluronan or a glycosaminoglycan having a water-chelating ability that is similar to hyaluronan; and (iii) a third hydrogel precursor component comprising at least one agent that promotes growth of a physiologically relevant tissue, wherein the hydrogel precursor solution polymerizes under suitable conditions to form a three-dimensional hydrogel that supports growth of a physiologically relevant tissue when at least one cell is cultured in the three-dimensional hydrogel. In some embodiments, the hydrogel precursor composition (e.g., solution) comprises: (c) at least one cell. In some embodiments, the hydrogel precursor composition comprises a transparent hydrogel precursor solution.

Any suitable aqueous medium can be used as a solvent for the hydrogel precursor solution. In some embodiments, the aqueous medium comprises water. In some embodiments, the aqueous medium comprises saline (e.g., phosphate buffered saline (PBS). In some embodiments, the aqueous medium comprises a cell culture medium. It should be appreciated that any culture medium described herein could be used as the cell culture medium. Other suitable media would be apparent to the skilled artisan. In some embodiments, the aqueous medium comprises a sugar solution. In some embodiments, the aqueous medium comprises a solvent (e.g., DMSO).

Generally, extracellular matrix proteins used as the first hydrogel precursor component of the hydrogel precursor solution are selected based on the composition of extracellular matrix proteins present in vivo in a tissue of interest. For example, for growth of physiologically relevant mammary tissue, extracellular matrix proteins present in human mammary tissue can be selected for use as the first hydrogel precursor component. In some embodiments, a combination of at least two, at least three, at least four, or at least five or more extracellular matrix proteins can be used as the first hydrogel precursor component. In some embodiments, the extracellular matrix protein comprises elastin. Exemplary extracellular matrix proteins include, without limitation, collagen, fibronectin, laminin, elastin, and fragments and subunits thereof. In some embodiments, the extracellular matrix protein is not elastin.

In some embodiments, the hydrogel precursor composition (e.g., solution) lacks a surfactant. In some embodiments, at least one of the extracellular matrix proteins is at least partially unfolded.

In some embodiments, the first hydrogel precursor component comprises the extracellular matrix protein collagen. In some embodiments, the first hydrogel precursor component comprises the extracellular matrix protein fibronectin. In some embodiments, the first hydrogel precursor component comprises the extracellular matrix protein laminin. In some embodiments, the first hydrogel precursor component comprises an extracellular matrix protein selected from the group consisting of collagen, fibronectin, and laminin. In some embodiments, the first hydrogel precursor component comprises two extracellular matrix proteins selected from the group consisting of collagen, fibronectin and laminin. In some embodiments, the first hydrogel precursor component comprises the extracellular matrix proteins collagen, fibronectin and laminin.

Any suitable form of collagen can be used as the first hydrogel precursor component. In some embodiments, the type of collagen used is based on the collagen present in vivo for the type of physiologically relevant tissue of interest. In some embodiments, the collagen comprises a soluble form of collagen. In some embodiments, the collagen comprises collagen type I. In some embodiments, the collagen comprises collagen type II. In some embodiments, the collagen comprises collagen type III. In some embodiments, the collagen comprises collagen type IV. In some embodiments, the collagen comprises collagen type V. In some embodiments, the collagen comprises collagen type VI. In some embodiments, the collagen comprises collagen type VII. In some embodiments, the collagen comprises collagen type VIII. In some embodiments, the collagen comprises rat tail collagen. In some embodiments, the collagen comprises Type I collagen, rat tail (commercially available from EMD Millipore, Billerica, Mass.). In some embodiments, the collagen comprises isolated collagen. In some embodiments, the collagen comprises recombinant collagen. In some embodiments, the collagen comprises human collagen.

In some embodiments, fragments and/or variants of any of the above forms of collagen can be used. In some embodiments, the collagen comprises a fragment or variant having an amino acid sequence that is at least 95%, at least 96%, at least 97%, at least 98%, or at least 99%, identical to any of the above forms of collagen. In some embodiments, the collagen comprises a variant of any of the above forms of collagen having at least one conservative amino acid substitution, at least two conservative amino acid substitutions, at least three conservative amino acid substitutions, at least four conservative amino acid substitutions, at least five conservative amino acid substitutions, at least six conservative amino acid substitutions, at least seven conservative amino acid substitutions, at least eight conservative amino acid substitutions, at least nine conservative amino acid substitutions, or at least 10 conservative amino acid substitutions. In some embodiments, the collagen comprises a variant of any of the above forms of collagen having at least one non-naturally occurring conservative amino acid substitution, at least two non-naturally occurring conservative amino acid substitutions, at least three non-naturally occurring conservative amino acid substitutions, at least four non-naturally occurring conservative amino acid substitutions, at least five non-naturally occurring conservative amino acid substitutions, at least six non-naturally occurring conservative amino acid substitutions, at least seven non-naturally occurring conservative amino acid substitutions, at least eight non-naturally occurring conservative amino acid substitutions, at least nine non-naturally occurring conservative amino acid substitutions, or at least 10 non-naturally occurring conservative amino acid substitutions. In some embodiments, the collagen comprises a fragment of any of the above forms of collagen in which at least one amino acid, at least two amino acids, at least three amino acids, at least four amino acids, at least five amino acids, at least six amino acids, at least seven amino acids, at least eight amino acids, at least nine amino acids, or at least ten amino acids have been deleted. In some embodiments, the deletions comprise N-terminal deletions. In some embodiments, the deletions comprise C-terminal deletions.

The hydrogel precursor composition (e.g., solution) can be formulated with different concentrations of collagen. Generally, the hydrogel precursor solutions can be formulated with effective amounts of collagen. In some embodiments, the collagen present in the hydrogel precursor solution ranges from between 0.1 mg/ml to 10.0 mg/ml. In some embodiments, the collagen is present in the hydrogel precursor solution at a concentration of between 0.5 mg/ml and 4.0 mg/ml. In some embodiments, the collagen is present at a concentration of 1.0 mg/ml. In some embodiments, the collagen is present at a concentration of 1.1 mg/ml. In some embodiments, the collagen is present at a concentration of 1.2 mg/ml. In some embodiments, the collagen is present at a concentration of 1.3 mg/ml. In some embodiments, the collagen is present at a concentration of 1.4 mg/ml. In some embodiments, the collagen is present at a concentration of 1.5 mg/ml. In some embodiments, the collagen is present at a concentration of 1.6 mg/ml. In some embodiments, the collagen is present at a concentration of 1.7 mg/ml. In some embodiments, the collagen is present at a concentration of 1.8 mg/ml. In some embodiments, the collagen is present at a concentration of 1.9 mg/ml. In some embodiments, the collagen is present at a concentration of 2.0 mg/ml.

Any suitable form of fibronectin can be used as the first hydrogel precursor component. In some embodiments, the type of fibronectin used is based on the fibronectin present in vivo for the type of physiologically relevant tissue of interest. In some embodiments, the fibronectin comprises a soluble form of fibronectin. In some embodiments, the fibronectin comprises plasma fibronectin. In some embodiments, the fibronectin comprises isolated fibronectin. In some embodiments, the fibronectin comprises recombinant fibronectin. In some embodiments, the fibronectin comprises human fibronectin. In some embodiments, the fibronectin comprises human plasma fibronectin (commercially available from Life Technologies, Waltham, Mass.).

In some embodiments, the fibronectin comprises a fragment or variant of any of the above forms of fibronectin. In some embodiments, the fibronectin comprises a fragment or variant having an amino acid sequence that is at least 95%, at least 96%, at least 97%, at least 98%, or at least 99%, identical to any of the above forms of fibronectin. In some embodiments, the fibronectin comprises a variant of any of the above forms of fibronectin having at least one conservative amino acid substitution, at least two conservative amino acid substitutions, at least three conservative amino acid substitutions, at least four conservative amino acid substitutions, at least five conservative amino acid substitutions, at least six conservative amino acid substitutions, at least seven conservative amino acid substitutions, at least eight conservative amino acid substitutions, at least nine conservative amino acid substitutions, or at least 10 conservative amino acid substitutions. In some embodiments, the fibronectin comprises a variant of any of the above forms of fibronectin having at least one non-naturally occurring conservative amino acid substitution, at least two non-naturally occurring conservative amino acid substitutions, at least three non-naturally occurring conservative amino acid substitutions, at least four non-naturally occurring conservative amino acid substitutions, at least five non-naturally occurring conservative amino acid substitutions, at least six non-naturally occurring conservative amino acid substitutions, at least seven non-naturally occurring conservative amino acid substitutions, at least eight non-naturally occurring conservative amino acid substitutions, at least nine non-naturally occurring conservative amino acid substitutions, or at least 10 non-naturally occurring conservative amino acid substitutions. In some embodiments, the fibronectin comprises a fragment of any of the above forms of fibronectin in which at least one amino acid, at least two amino acids, at least three amino acids, at least four amino acids, at least five amino acids, at least six amino acids, at least seven amino acids, at least eight amino acids, at least nine amino acids, or at least ten amino acids have been deleted. In some embodiments, the deletions comprise N-terminal deletions. In some embodiments, the deletions comprise C-terminal deletions.

The hydrogel precursor composition (e.g., solution) can be formulated with different concentrations of fibronectin. Generally, the hydrogel precursor solutions can be formulated with effective amounts of fibronectin. In some embodiments, the fibronectin present in the hydrogel precursor solution ranges from between 1 μg/mL to 100 μg/mL. In some embodiments, the fibronectin is present in the hydrogel precursor solution at a concentration of between 1 μg/mL to 100 μg/mL. In some embodiments, the fibronectin is present in the hydrogel precursor solution at a concentration of between 10 μg/mL to 50 μg/mL. In some embodiments, the fibronectin is present in the hydrogel precursor solution at a concentration of between 15 μg/mL to 35 μg/mL. In some embodiments, the fibronectin is present at a concentration of 15 μg/mL. In some embodiments, the fibronectin is present at a concentration of 16 μg/mL. In some embodiments, the fibronectin is present at a concentration of 17 μg/mL. In some embodiments, the fibronectin is present at a concentration of 18 μg/mL. In some embodiments, the fibronectin is present at a concentration of 19 μg/mL. In some embodiments, the fibronectin is present at a concentration of 20 μg/mL. In some embodiments, the fibronectin is present at a concentration of 21 μg/mL. In some embodiments, the fibronectin is present at a concentration of 22 μg/mL. In some embodiments, the fibronectin is present at a concentration of 23 μg/mL. In some embodiments, the fibronectin is present at a concentration of 24 μg/mL. In some embodiments, the fibronectin is present at a concentration of 25 μg/mL.

Any suitable form of laminin can be used as the first hydrogel precursor component. In some embodiments, the type of laminin used is based on the laminin present in vivo for the type of physiologically relevant tissue of interest. In some embodiments, the laminin comprises a soluble form of laminin. In some embodiments, the laminin comprises isolated laminin. In some embodiments, the laminin comprises recombinant laminin. In some embodiments, the laminin comprises human laminin. In some embodiments, the laminin comprises mouse laminin. In some embodiments, the laminin comprises laminin isolated from Engelbreth-Holm-Swarm (EHS) sarcoma cells (commercially available from Life Technologies, Waltham, Mass.). In some embodiments, the laminin comprises a fragment or variant of any of the above forms of laminin. In some embodiments, the laminin is not functionalized. In some embodiments, the laminin is not conjugated to the hydrogel via a linker.

In some embodiments, the laminin comprises a fragment or variant having an amino acid sequence that is at least 95%, at least 96%, at least 97%, at least 98%, or at least 99%, identical to any of the above forms of laminin. In some embodiments, the laminin comprises a variant of any of the above forms of laminin having at least one conservative amino acid substitution, at least two conservative amino acid substitutions, at least three conservative amino acid substitutions, at least four conservative amino acid substitutions, at least five conservative amino acid substitutions, at least six conservative amino acid substitutions, at least seven conservative amino acid substitutions, at least eight conservative amino acid substitutions, at least nine conservative amino acid substitutions, or at least 10 conservative amino acid substitutions. In some embodiments, the laminin comprises a variant of any of the above forms of laminin having at least one non-naturally occurring conservative amino acid substitution, at least two non-naturally occurring conservative amino acid substitutions, at least three non-naturally occurring conservative amino acid substitutions, at least four non-naturally occurring conservative amino acid substitutions, at least five non-naturally occurring conservative amino acid substitutions, at least six non-naturally occurring conservative amino acid substitutions, at least seven non-naturally occurring conservative amino acid substitutions, at least eight non-naturally occurring conservative amino acid substitutions, at least nine non-naturally occurring conservative amino acid substitutions, or at least 10 non-naturally occurring conservative amino acid substitutions. In some embodiments, the laminin comprises a fragment of any of the above forms of laminin in which at least one amino acid, at least two amino acids, at least three amino acids, at least four amino acids, at least five amino acids, at least six amino acids, at least seven amino acids, at least eight amino acids, at least nine amino acids, or at least ten amino acids have been deleted. In some embodiments, the deletions comprise N-terminal deletions. In some embodiments, the deletions comprise C-terminal deletions.

The hydrogel precursor composition (e.g., solution) can be formulated with different concentrations of laminin. Generally, the hydrogel precursor solutions can be formulated with effective amounts of laminin. In some embodiments, the laminin present in the hydrogel precursor solution ranges from between 1 μg/mL to 100 μg/mL. In some embodiments, the laminin is present in the hydrogel precursor solution at a concentration of between 20 μg/mL to 60 μg/mL. In some embodiments, the laminin is present in the hydrogel precursor solution at a concentration of between 30 μg/mL to 50 μg/mL. In some embodiments, the laminin is present at a concentration of 35 μg/mL. In some embodiments, the laminin is present at a concentration of 36 μg/mL. In some embodiments, the laminin is present at a concentration of 37 μg/mL. In some embodiments, the laminin is present at a concentration of 38 μg/mL. In some embodiments, the laminin is present at a concentration of 39 μg/mL. In some embodiments, the laminin is present at a concentration of 40 μg/mL. In some embodiments, the laminin is present at a concentration of 41 μg/mL. In some embodiments, the laminin is present at a concentration of 42 μg/mL. In some embodiments, the laminin is present at a concentration of 43 μg/mL. In some embodiments, the laminin is present at a concentration of 44 μg/mL. In some embodiments, the laminin is present at a concentration of 45 μg/mL.

In some embodiments, the extracellular matrix protein is pH neutralized. For example, in some embodiments, a starting solution of extracellular matrix protein is provided in the neutral pH range (e.g., 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, and any values and ranges therein) at a concentration of between 1 mM and 100 mM (e.g., 1 mM . . . 2 mM . . . 3 mM . . . 4 mM . . . 5 mM . . . 6 mM . . . 7 mM . . . 8 mM . . . 9 mM . . . 10 mM . . . 15 mM . . . 20 mM . . . 30 mM . . . 50 mM . . . 75 mM . . . 100 mM, and any values and ranges therein). In some embodiments, the pH of an acidic starting solution of extracellular matrix protein (e.g., collagen) is adjusted by the addition of a suitable base (e.g., NaOH) (or acid if the protein solution is basic) to produce a pH neutral hydrogelation solution that is to be used as part of the first hydrogel precursor component. In some embodiments, the starting solution of extracellular matrix protein (e.g., collagen) is provided in a neutral pH range that is near but above or below physiological pH of a tissue of interest in vivo (e.g., mammary tissue, e.g., human mammary tissue), e.g., the extracellular matrix protein (e.g., collagen) is pH neutralized to a neutral pH of 0.1, 0.2, 0.3, 0.4, 0.5, 0.6, 0.7, 0.8, 0.9, or 1 less than or greater than the physiological pH of the tissue of interest in vivo. In some embodiments, the pH is adjusted gradually, for example, by dropwise addition of base (or acid). Gradual pH adjustment may take place over 1 minute . . . 5 minutes . . . 10 minutes . . . 20 minutes . . . 30 minutes . . . 1 hour . . . 2 hours, or more. In other embodiments, the pH is adjusted rapidly, for example, by rapid addition of a volume of base (or acid) (e.g., 1/100, 1/50, 1/25, 1/10, ⅛, ¼, ½, equal volume) to form the neutral starting solution. Rapid pH adjustment may take place in less than 1 minute . . . 30 seconds . . . 20 seconds . . . 10 seconds . . . 5 seconds . . . 2 seconds . . . 1 second, or less.

In some embodiments, at least one, at least two, or at least three of the extracellular matrix proteins used to form the first hydrogel precursor component are pre-treated before dissolving them in the aqueous media to form the hydrogel precursor solution. Generally, structural carbohydrate (e.g., glycosaminoglycan) used as the second hydrogel precursor component of the hydrogel precursor solution is selected based on the composition of structural carbohydrates present in vivo in a tissue of interest. For example, for growth of physiologically relevant mammary tissue, structural carbohydrates present in human mammary tissue can be selected for use as the second hydrogel precursor component. In some embodiments, the second hydrogel precursor component comprises hyaluronan. In some embodiments, the second hydrogel precursor component comprises a glycosaminoglycan. Glycosaminoglycans are polysaccharides containing amino sugars as a component. Examples of glycosaminoglycans include, without limitation, hyaluronic acid, chondroitin sulfate, dermatan sulfate, keratin sulfate, dextran sulfate, heparin sulfate, heparin, glucuronic acid, iduronic acid, galactose, galactosamine, and glucosamine and analogs or variants thereof. In some embodiments, the second hydrogel precursor component comprises a glycosaminoglycan that has a water-chelating ability that is similar to hyaluronan.

Those skilled in the art will appreciate that glycosaminoglycans (and other structural carbohydrates) can be selected as having a water-chelating ability that is similar to hyaluronan, for example, by constructing a three-dimensional hydrogel that is the same in all respects except that it uses a substituted carbohydrate or glycosaminoglycan other than hyaluronan, and then the swelling ratio and/or elastic modulus of the resulting three-dimensional hydrogel can be measured to identify three-dimensional hydrogels that exhibit swelling ratios and elastic moduli that are similar to the three-dimensional hydrogels constructed using hyaluronan. Substituted carbohydrates or glycosaminoglycans resulting in three-dimensional hydrogels exhibit swelling ratios and elastic moduli that are similar to the three-dimensional hydrogels constructed using hyaluronan are considered to be structural carbohydrates and glycosaminoglyans having a water-chelating ability that is similar to hyaluronan. Other methods of selecting structural carbohydrates and glycosaminoglycans having a water-chelating ability similar to that of hyaluronan are apparent to the skilled artisan.

In some embodiments, the second hydrogel precursor component comprises a non-sulfated glycosaminoglycan. In some embodiments, the glycosaminoglycan is not chondroitin sulfate. In some embodiments, the glycosaminoglycan is not dermatan sulfate. In some embodiments, the glycosaminoglycan is not keratin sulfate. In some embodiments, the glycosaminoglycan is not heparin sulfate. In some embodiments, the glycosaminoglycan is not dextran sulfate. In some embodiments, the glycosaminoglycan is not heparin. In some embodiments, the glycosaminoglycan is not glucuronic acid. In some embodiments, the glycosaminoglycan is not iduronic acid. In some embodiment, the glycosaminoglycan is not galactose. In some embodiments, the glycosaminoglycan is not galactosamine. In some embodiments, the glycosaminoglycan is not glucosamine. In some embodiments, the second hydrogel precursor component comprises a hydrophilic glycosaminoglycan.

Any suitable form of hyaluronan can be used as the second hydrogel precursor component. In some embodiments, the type of hyaluronan used is based on the hyaluronan present in vivo for the type of physiologically relevant tissue of interest. In some embodiments, the hyaluronan comprises a soluble form of hyaluronan. In some embodiments, the hyaluronan comprises isolated hyaluronan. In some embodiments, the laminin comprises recombinant hyaluronan. In some embodiments, the hyaluronan comprises human hyaluronan. In some embodiments, the hyaluronan has a molecular weight ranging from 25 kDA to 1000 kDA. In some embodiments, the hyaluronan comprises a mixture of a low molecular weight hyaluronan and a high molecular weight hyaluronan. In some embodiments, the low molecular weight hyaluronan has a molecular weight of 100 kDA, 110 kDA, 115 kDA, 120 kDA, 125 kDA, 130 kDA, 135 kDA, 140 kDA, 145 kDA, 146 kDA, 147 kDA, 148 kDA, 149 kDA, 150 kDA, 151 kDA, 152 kDA, 153 kDA, 154 kDA, 155 kDA, 160 kDA, 165 kDA, 170 kDA, 175 kDA, 180 kDA, 185 kDA, 190 kDA and 200 kDA. In some embodiments, the low molecular weight hyaluronan has a molecular weight of 150 kDA. In some embodiments, the high molecular weight hyaluronan has a molecular weight of about 400 kDA, 410 kDA, 415 kDA, 420 kDA, 425 kDA, 430 kDA, 435 kDA, 440 kDA, 445 kDA, 450 kDA, 460 kDA, 465 kDA, 470 kDA, 475 kDA, 480 kDA, 485 kDA, 490 kDA, 491 kDA, 492 kDA, 493 kDA, 495 kDA, 496 kDA, 497 kDA, 498 kDA, 499 kDA, 500 kDA, 501 kDA, 502 kDA, 503 kDA, 504 kDA, 505 kDA, 506 kDA, 507 kDA, 508 kDA, 509 kDA, 510 kDA, 515 kDA, 520 kDA, 525 kDA, 530 kDA, 535 kDA, 540 kDA, 545 kDA, 550 kDA, 555 kDA, 560 kDA, 565 kDA, 570 kDA, 575 kDA, 580 kDA, 585 kDA, 590 kDA, 595 kDA, and 600 kDA. In some embodiments, the high molecular weight hyaluronan comprises a molecular weight of 500 kDa. In some embodiments, the low molecular weight hyaluronan comprises a molecular weight of 150 kDA and the high molecular weight hyaluronan comprises a molecular weight of 500 kDA. In some embodiments, the hyaluronan comprises hyaluronan (commercially available from Sigma Aldrich, St. Louis, Mo.). In some embodiments, the hyaluronan comprises an analog or derivative of any of the above forms of hyaluronan.

The hydrogel precursor solution can be formulated with different concentrations of hyaluronan or a glycosaminoglycan having a water-chelating ability similar to hyaluronan. Generally, the hydrogel precursor solutions can be formulated with effective amounts of hyaluronan or glycosaminoclygan having a water-chelating ability similar to hyaluronan. In some embodiments, the hyaluronan present in the hydrogel precursor solution ranges from between 1 μg/mL to 50 μg/mL. In some embodiments, the hyaluronan is present in the hydrogel precursor solution at a concentration of between 5 μg/mL to 25 μg/mL. In some embodiments, the hyaluronan is present in the hydrogel precursor solution at a concentration of between 10 μg/mL to 20 μg/mL. In some embodiments, the hyaluronan is present at a concentration of 5 μg/mL. In some embodiments, the hyaluronan is present at a concentration of 6 μg/mL. In some embodiments, the hyaluronan is present at a concentration of 7 μg/mL. In some embodiments, the hyaluronan is present at a concentration of 8 μg/mL. In some embodiments, the hyaluronan is present at a concentration of 9 μg/mL. In some embodiments, the hyaluronan is present at a concentration of 10 μg/mL. In some embodiments, the hyaluronan is present at a concentration of 11 μg/mL. In some embodiments, the hyaluronan is present at a concentration of 12 μg/mL. In some embodiments, the hyaluronan is present at a concentration of 13 μg/mL. In some embodiments, the hyaluronan is present at a concentration of 14 μg/mL. In some embodiments, the hyaluronan is present at a concentration of 15 μg/mL.

In some embodiments, agents that promote growth (and/or differentiation) of a physiologically relevant tissue used as the third hydrogel precursor component of the hydrogel precursor composition can be selected based on the types of agents present in vivo for stimulating growth of a tissue of interest. For example, for growth of physiologically relevant mammary tissue, agents known to stimulate growth of human mammary tissue in vivo can be selected for use as the third hydrogel precursor component. Examples of at least one agent that promotes growth of a physiologically relevant issue for use as the third hydrogel precursor component include cytokines, growth factors, morphogens, and steroid hormones. In some embodiments, combinations of at least two, at least three, at least four, or at least five agents that promote growth of a physiologically relevant tissue can be used as the third hydrogel precursor component.

Exemplary cytokines include, without limitation, erythropoietin, granulocyte-macrophage colony stimulating factor, granulocyte colony stimulating factor, lipopolysaccharide, macrophage colony stimulating factor, thrombopoietin, stem cell factor, interleukin-1, interleukin-2, interleukin-3, interleukin-6, interleukin-7, interleukin-15, Flt3L, leukemia inhibitory factor, insulin-like growth factor, insulin, or any functional variant or fragment thereof. In some embodiments, the cytokine is not erythropoietin. In some embodiments, the cytokine is not granulocyte-macrophage colony stimulating factor. In some embodiments, the cytokine is not granulocyte colony stimulating factor. In some embodiments, the cytokine is not lipopolysaccharide. In some embodiments, the cytokine is not macrophage colony stimulating factor. In some embodiments, the cytokine is not thrombopoietin. In some embodiments, the cytokine is not stem cell factor. In some embodiments, the cytokine is not interleukin-1. In some embodiments, the cytokine is not interleukin-2. In some embodiments, the cytokine is not interleukin-3. In some embodiments, the cytokine is not interleukin-6. In some embodiments, the cytokine is not interleukin-7. In some embodiments, the cytokine is not interleukin-15. In some embodiments, the cytokine is not Flt3L. In some embodiments, the cytokine is not leukemia inhibitory factor. In some embodiments, the cytokine is not insulin like growth factor.

Exemplary growth factors include, without limitation fibroblast growth factor, epidermal growth factor, insulin-like growth factor 1, platelet-derived growth factor, nerve growth factor, brain-derived neurotrophic factor, neurotrophin 3 (NT-3), neurotrophin 4 (NT-4), transforming growth factor beta, or any functional variant or fragment thereof. In some embodiments, the growth factor is not fibroblast growth factor. In some embodiments, the growth factor is not insulin-like growth factor 1. In some embodiments, the growth factor is not platelet-derived growth factor. In some embodiments, the growth factor is not nerve growth factor. In some embodiments, the growth factor is not transforming growth factor beta. In some embodiments, the growth factor is not brain-derived neurotrophic factor. In some embodiments, the growth factor is not NT-3. In some embodiments, the growth factor is not NT-4.

Exemplary morphogens include, without limitation, transforming growth factor beta (TGF-beta), Hedgehog/Sonic Hedgehog, Wingless/Wnt, epidermal growth factor (EGF), and fibroblast growth factor (FGF), or any functional variant or fragment thereof. In some embodiments, the morphogen is not TGF-beta. In some embodiment, the morphogen is not Hedgehog/Sonic Hedgehog. In some embodiments, the morphogen is not Wingless/Wnt. In some embodiments, the morphogen is not FGF.

Exemplary steroid hormones include, without limitation, corticosteroids and sex steroids. Suitable corticosteroids include, for example, glucocorticoids and mineralocorticoids. Suitable sex steroids include, for example, androgens, estrogens, and progestogens. In some embodiments, the steroid hormone comprises a glucocorticoid or mineralocorticoid selected from the group consisting of aldosterone, alclometasone, beclomethasone, betamethasone, cortisol, prednisone, prednisolone, methylprednisolone, dexamethasone, fludrocortisone, fludrocortisone acetate, triamcinolone, cortisone, deoxycorticosterone acetate, and analogs thereof. In some embodiments, the steroid hormone is hydrocortisone. In some embodiments, the steroid hormone comprises dihydrotachysterol. In some embodiments, the steroid hormone comprises an androgen selected from the group consisting of apoptone, oxandrolone, oxabolone, testosterone, nandrolone. In some embodiments, the steroid hormone comprises an estrogen selected from the group consisting of diethylstilbestrol and beta estradiol. In some embodiments, the steroid hormone comprises a progestin selected from the group consisting of danazol, norethisterone, medroxyprogesterone acetate, and 17-hydroxyprogesterone caproate.

In some embodiments, at least one agent that promotes growth of physiologically relevant tissue used as the third hydrogel precursor component comprises epidermal growth factor (EGF) or a functional variant thereof. In some embodiments, the EGF comprises isolated EGF. In some embodiments, the EGF comprises recombinant EGF. In some embodiments, the EGF comprises human EGF. In some embodiments, the at least one agent comprises an agent that activates EGF receptor signaling (e.g., betacellulin). In some embodiments, the at least one agent comprises EGF (commercially available from Lonza CC-4021G, CC-4031G, and CC-4017G respectively).

The hydrogel precursor compositions (e.g., solutions) can be formulated with different concentrations of EGF or a functional variant or mimetic thereof. Generally, the hydrogel precursor solutions can be formulated with effective amounts of EGF or a functional variant, analog, or mimetic thereof. In some embodiments, the EGF or functional variant, analog, or mimetic thereof is present at a concentration of between 1 ng/mL and 100 ng/mL.

In some embodiments, at least one agent that promotes growth of physiologically relevant tissue used as the third hydrogel precursor component comprises insulin or a functional variant or fragment thereof. In some embodiments, the insulin comprises isolated insulin. In some embodiments, the insulin comprises recombinant insulin. In some embodiments, the insulin comprises human insulin. In some embodiments, the insulin comprises at least one agent that activates the insulin receptor (e.g., insulin receptor agonists or insulin mimetic). In some embodiments, the insulin mimetic comprises a chaetochromin derivative. In some embodiments, the insulin mimetic comprises chaetochromin derivative 4548-G05 as described by Giang et al. (“Identification of a small molecular insulin receptor agonist with potent antidiabetes activity,” Diabetes. 2014; 63(4): 1394-409). In some embodiments, the insulin receptor agonist comprises an antibody, e.g., monoclonal antibody that binds with high-affinity to the insulin receptor. In some embodiments, the insulin receptor agonist comprises XMetA as described by Vigneri et al. (“Selective Insulin Receptor Modulators (SIRM): A New Class of Antidabetes Drugs?,” Diabetes. 2012; 61(5): 984-985). In some embodiments, the insulin receptor agonist comprises peptide 5961 present at a nanomolar concentration range of 1-10 nM (see, e.g., Knudsen et al., “Agonism and Antagonism at the Insulin Receptor,” PLoS ONE. 2012; 7(12): e51972). In some embodiments, the insulin receptor agonist comprises a peptide agonist of the insulin receptor, for example, an optimized insulin receptor peptide agonist, such as 5519 (see, e.g., Schaffer et al., “Assembly of high-affinity insulin receptor agonists and antagonists from peptide building blocks,” PNAS. 2003; 100(8): 4435-4439).

The hydrogel precursor composition (e.g., solution) can be formulated with different concentrations of insulin or a functional variant, analog, or mimetic thereof. Generally, the hydrogel precursor solutions can be formulated with effective amounts of insulin or a functional variant, analog, or mimetic thereof. In some embodiments, the insulin or functional variant or mimetic thereof is present at a concentration of between 1 μg/mL and 100 μg/mL.

In some embodiments, at least one agent that promotes growth of physiologically relevant tissue used as the third hydrogel precursor component comprises hydrocortisone or a derivative or an analog thereof. In some embodiments, the hydrocortisone comprises hydrocortisone aceponate. In some embodiments, the hydrocortisone comprises hydrocortisone acetate. In some embodiments, the hydrocortisone comprises hydrocortisone butyrate. In some embodiments, the hydrocortisone comprises hydrocortisone cypionate. In some embodiments, the hydrocortisone comprises hydrocortisone probutate. In some embodiments, the hydrocortisone comprises hydrocortisone sodium phosphate. In some embodiments, the hydrocortisone comprises hydrocortisone sodium succinate. In some embodiments, the hydrocortisone comprises hydrocortisone valerate.

The hydrogel precursor composition (e.g., solution) can be formulated with different concentrations of hydrocortisone or a derivative or analog thereof. Generally, the hydrogel precursor solutions can be formulated with effective amounts of hydrocortisone or a derivative or analog thereof. In some embodiments, the hydrocortisone or derivative or analog thereof is present at a concentration of between 50 ng/mL and 5 μg/mL.

In some embodiments, the third hydrogel precursor component comprises at least two agents that promote growth of physiologically relevant tissue selected from the group consisting of EGF, insulin and hydrocortisone. In some embodiments, the third hydrogel precursor component comprises at least two agents that promote growth of physiologically relevant tissue selected from the group consisting of EGF or an agent that activates EGF receptor signaling, insulin or an insulin receptor agonist or insulin mimetic, and hydrocortisone or an analog or derivative thereof. In some embodiments, the third hydrogel precursor component comprises EGF, insulin and hydrocortisone. In some embodiments, the third hydrogel precursor component comprises EGF or an agent that activates EGF receptor signaling, insulin or an insulin receptor agonist or insulin mimetic, and hydrocortisone or an analog or derivative thereof.

In an exemplary embodiment, a hydrogel precursor solution for forming a three-dimensional hydrogel that supports growth of a physiologically relevant tissue when at least one cell or at least one cluster of cells is cultured in the three-dimensional hydrogel, the hydrogel precursor solution consists of, consists essentially of, or comprises: (a) an aqueous medium; (b) at least three hydrogel precursor components dissolved in the aqueous medium to form a hydrogel precursor solution for forming a three-dimensional hydrogel that supports growth of physiologically relevant tissue, wherein the at least three hydrogel precursor components comprise: (i) a first hydrogel precursor component comprising extracellular matrix proteins collagen, fibronectin, and laminin; (ii) a second hydrogel precursor component comprising hyaluronan or a glycosaminoglycan having a water-chelating ability that is similar to hyaluronan; and (iii) a third hydrogel precursor component comprising EGF, insulin, and hydrocortisone, wherein the hydrogel precursor solution polymerizes under suitable conditions to form a three-dimensional hydrogel that supports growth of a physiologically relevant tissue when at least one cell is cultured in the three-dimensional hydrogel.

In an exemplary embodiment, a hydrogel precursor solution for forming a three-dimensional hydrogel that supports growth of physiologically relevant mammary tissue when at least one mammary epithelial cell or at least one cluster of mammary epithelial cells is cultured in the three-dimensional hydrogel, the hydrogel precursor solution consists of, consists essentially of, or comprises: (a) an aqueous medium; (b) at least three hydrogel precursor components dissolved in the aqueous medium to form a hydrogel precursor solution for forming a three-dimensional hydrogel that supports growth of physiologically relevant mammary tissue, wherein the at least three hydrogel precursor components comprise: (i) a first hydrogel precursor component comprising extracellular matrix proteins collagen, fibronectin, and laminin; (ii) a second hydrogel precursor component comprising hyaluronan or a glycosaminoglycan having a water-chelating ability that is similar to hyaluronan; and (iii) a third hydrogel precursor component comprising EGF, insulin, and hydrocortisone, wherein the hydrogel precursor solution polymerizes under suitable conditions to form a three-dimensional hydrogel that supports growth of physiologically relevant mammary tissue when at least one mammary epithelial cell or at least one cluster of mammary epithelial cells is cultured in the three-dimensional hydrogel.

In an exemplary embodiment, a hydrogel precursor solution for forming a three-dimensional hydrogel that supports growth of physiologically relevant tumor tissue when at least one cancerous epithelial cell or at least one cluster of cancerous epithelial cells is cultured in the three-dimensional hydrogel, the hydrogel precursor solution consists of, consists essentially of, or comprises: (a) an aqueous medium; (b) at least three hydrogel precursor components dissolved in the aqueous medium to form a hydrogel precursor solution for forming a three-dimensional hydrogel that supports growth of physiologically relevant tumor tissue, wherein the at least three hydrogel precursor components comprise: (i) a first hydrogel precursor component comprising extracellular matrix proteins collagen, fibronectin, and laminin; (ii) a second hydrogel precursor component comprising hyaluronan or a glycosaminoglycan having a water-chelating ability that is similar to hyaluronan; and (iii) a third hydrogel precursor component comprising EGF, insulin, and hydrocortisone, wherein the hydrogel precursor solution polymerizes under suitable conditions to form a three-dimensional hydrogel that supports growth of physiologically relevant tumor tissue when at least one cancerous epithelial cell or at least one cluster of cancerous epithelial cells is cultured in the three-dimensional hydrogel.

In some aspects, hydrogels described herein may be used to culture non-epithelial cells, e.g., non-epithelial cancer cells. In an exemplary embodiment, a hydrogel precursor solution for forming a three-dimensional hydrogel that supports growth of physiologically relevant tumor tissue when at least one cancerous non-epithelial cell or at least one cluster of cancerous non-epithelial cells is cultured in the three-dimensional hydrogel, the hydrogel precursor solution consists of, consists essentially of, or comprises: (a) an aqueous medium; (b) at least three hydrogel precursor components dissolved in the aqueous medium to form a hydrogel precursor solution for forming a three-dimensional hydrogel that supports growth of physiologically relevant tumor tissue, wherein the at least three hydrogel precursor components comprise: (i) a first hydrogel precursor component comprising extracellular matrix proteins collagen, fibronectin, and laminin; (ii) a second hydrogel precursor component comprising hyaluronan or a glycosaminoglycan having a water-chelating ability that is similar to hyaluronan; and (iii) a third hydrogel precursor component comprising EGF, insulin, and hydrocortisone, wherein the hydrogel precursor solution polymerizes under suitable conditions to form a three-dimensional hydrogel that supports growth of physiologically relevant tumor tissue when at least one cancerous non-epithelial cell or at least one cluster of cancerous non-epithelial cells is cultured in the three-dimensional hydrogel.

In some embodiments, a hydrogel precursor solution for forming a three-dimensional hydrogel that supports growth of physiologically relevant tissue when at least one non-epithelial cell or at least one cluster of non-epithelial cells is cultured in the three-dimensional hydrogel, the hydrogel precursor solution consists of, consists essentially of, or comprises: (a) an aqueous medium; (b) at least three hydrogel precursor components dissolved in the aqueous medium to form a hydrogel precursor solution for forming a three-dimensional hydrogel that supports growth of physiologically relevant tumor tissue, wherein the at least three hydrogel precursor components comprise: (i) a first hydrogel precursor component comprising extracellular matrix proteins collagen, fibronectin, and laminin; (ii) a second hydrogel precursor component comprising hyaluronan or a glycosaminoglycan having a water-chelating ability that is similar to hyaluronan; and (iii) a third hydrogel precursor component comprising one or more growth factors capable of supporting the growth and/or differentiation of a non-epithelial cell, wherein the hydrogel precursor solution polymerizes under suitable conditions to form a three-dimensional hydrogel that supports growth of physiologically relevant tissue when at least one non-epithelial cell or at least one cluster of non-epithelial cells is cultured in the three-dimensional hydrogel. In some embodiments the physiologically relevant tissue comprises nervous system tissue (e.g., isolated from brain, from a ganglion, from a sensory organ, from a nerve tract or plexus, or from a nerve). In some embodiments, at least one agent that promotes growth and/or differentiation of physiologically relevant tissue (e.g., nervous system tissue) used as the third hydrogel precursor component comprises nerve growth factor (NGF) or a functional variant thereof. In some embodiments, the NGF comprises isolated NGF. In some embodiments, the NGF comprises recombinant NGF. In some embodiments, the NGF comprises human NGF. In some embodiments, the at least one agent comprises an agent that activates NGF receptor signaling (e.g., an agonist of one or more of the NGF receptors TrkA, TrkB, and TrkC such as gambogic acid, amytryptiline). In some embodiments, the at least one agent comprises NGF (e.g., recombinant Human β-NGF such as is commercially available from Peprotech (450-01) or NGF-2.5S from mouse, such as is commercially available from Sigma (N6009). One of ordinary skill in the art appreciates that “NGF” used in the hydrogel described herein comprises beta NGF, the functionally active signaling subunit of the NGF complex. In some embodiments, the agent comprises a derivative or analog of NGF. The NGF can be added to the culture medium or the three-dimensional hydrogel in various concentrations. As will be appreciated, NGF is a member of the NGF family, which includes NGF, BDNF, NT-3, and NT-4. NGF, BDNF, NT-3, and NT-4 may be referred to as neurotrophins. In some aspects, the presently disclosed subject matter contemplates the presence of effective amounts of NGF (and/or other NGF family member(s)) or other agents that activate NGF receptor signaling in the culture medium and/or in the three-dimensional hydrogel. In some embodiments, the NGF (and/or other NGF family member (s)) is present at a concentration of between 0.1 ng/mL and 1000 ng/mL. For example, in some embodiments, the NGF (and/or other NGF family member (s)) is present at a concentration of between 1 ng/ml and 10 ng/ml or between 10 ng/ml and 100 ng/ml, e.g., 20 ng/ml-50 ng/ml or 50 ng/ml-100 ng/ml. In certain embodiments the concentration is 40 ng/ml.

In some embodiments, a hydrogel or hydrogel precursor solution that comprises NGF and/or one or more other NGF family members or other agents that activate NGF receptor signaling does not comprise insulin. In some embodiments, a hydrogel or hydrogel precursor solution that comprises NGF and/or one or more other NGF family members or other agents that activate NGF receptor signaling does not comprise hydrocortisone. In some embodiments, a hydrogel or hydrogel precursor solution that comprises NGF and/or one or more other NGF family members or other agents that activate NGF receptor signaling does not comprise a corticosteroid. In some embodiments, a hydrogel or hydrogel precursor solution that comprises NGF and/or one or more other NGF family members or other agents that activate NGF receptor signaling does not comprise EGF. In some embodiments, a hydrogel precursor solution or hydrogel that comprises NGF and/or one or more other NGF family members or other agents that activate NGF receptor signaling does not comprise insulin, does not comprise hydrocortisone, and does not comprise EGF. In some embodiments, a hydrogel or hydrogel precursor solution comprises NGF (and/or, in some embodiments, one or more other NGF family members) as the only growth factor. In some embodiments, a hydrogel that is formed from a hydrogel precursor solution that comprises NGF and/or one or more other NGF family members or other agents that activate NGF receptor signaling (and optionally lacks any one or more other growth factors as described herein, e.g., insulin, hydrocortisone, and/or EGF), is used to culture neurons. Exemplary types of neurons are described elsewhere herein. In some embodiments the culture medium used to culture such neurons comprises NGF and/or one or more other NGF family members or other agents that activate NGF receptor signaling (and optionally lacks any one or more other growth factors described herein). In some embodiments the culture medium used to culture such neurons comprises NGF and/or one or more other NGF family members or other agents that activate NGF receptor signaling comprises one or more other growth factors described herein, e.g., EGF, hydrocortisone, and/or insulin.

In some embodiments a hydrogel and/or culture medium may comprise an agent that promotes myelin production. In some embodiments the agent comprises ascorbic acid. In some embodiments such a hydrogel may be used to co-culture neurons and glial cells. In some embodiments an agent that promotes myelin production may be present in a hydrogel precursor solution addition to at least one agent that promotes growth and/or differentiation of nervous system tissue (e.g., NGF and/or one or more other NGF family members or other agents that activate NGF receptor signaling).

### II. Kits

Aspects of the presently disclosed subject matter relate to kits useful for practicing the method of the presently disclosed subject matter. In general, a presently disclosed kit contains some or all of the components, reagents, supplies, and the like to practice a method according to the presently disclosed subject matter. In some embodiments, the term “kit” refers to any intended article of manufacture (e.g., a package or a container) comprising a hydrogel precursor solution disclosed herein, or a component of the hydrogel precursor solution, and a particular set of instructions for polymerizing the hydrogel precursor solution (or components of the hydrogel precursor solution) to form a three-dimensional hydrogel that supports growth of a physiologically relevant tissue when at least one cell is cultured in the three-dimensional hydrogel. The kit can be packaged in a divided or undivided container, such as a carton, bottle, ampule, tube, etc. The presently disclosed hydrogel precursor components can be packaged in dried, lyophilized, or liquid form. Additional components provided can include vehicles for reconstitution of dried components. Preferably all such vehicles are sterile and apyrogenic so that they are suitable for injection into a subject without causing adverse reactions.

In some embodiments, the presently disclosed subject matter provides a kit for forming a three-dimensional hydrogel that supports growth of a physiologically relevant tissue when at least one cell is cultured in the three-dimensional hydrogel, the kit consisting of, consisting essentially of, or comprising: (a) a hydrogel precursor composition comprising: (i) a first hydrogel precursor component comprising an extracellular matrix protein selected from the group consisting of collagen, fibronectin, and laminin; (ii) a second hydrogel precursor component comprising hyaluronan or a glycosaminoglycan having a water-chelating ability that is similar to hyaluronan; and (iii) optionally a third hydrogel precursor component comprising at least one agent that promotes growth of a physiologically relevant tissue; and (b) instructions for polymerizing the hydrogel precursor composition under suitable conditions to form a three-dimensional hydrogel that supports growth of a physiologically relevant tissue when at least one cell is cultured in the three-dimensional hydrogel.

In some embodiments, the kit further includes an aqueous medium or instructions to add the first, second and third hydrogel precursor component, if present, to an aqueous medium to form a hydrogel precursor solution, and then instructions for polymerizing the hydrogel precursor solution to form the three-dimensional hydrogel.

In some embodiments, the hydrogel precursor composition comprises a hydrogel precursor solution. In some embodiments, the hydrogel precursor solution comprises the first hydrogel precursor component, the second hydrogel precursor component, and the third hydrogel precursor component, if present, dissolved in an aqueous medium.

The first hydrogel precursor component, the second hydrogel precursor component, and the third hydrogel precursor component, if present, can be provided in the same container or in different containers of the kit.

In some embodiments, the first hydrogel precursor component comprises at least two extracellular matrix proteins selected from the group consisting of collagen, fibronectin, and laminin. In some embodiments, the first hydrogel precursor component comprises at least three extracellular matrix proteins selected from the group consisting of collagen, fibronectin and laminin.

In some embodiments, at least one agent that promotes growth of the physiologically relevant tissue is provided in the kit. In some embodiments, at least two agents that promote growth of the physiologically relevant tissue are provided in the kit. In some embodiments, at least three agents that promote growth of the physiologically relevant tissue are provided in the kit.

In some embodiments, the third hydrogel precursor component is provided in the kit, wherein the third hydrogel precursor component comprises at least one agent that promotes growth of the physiologically relevant tissue selected from the group consisting of EGF, insulin and hydrocortisone. In some embodiments, the third hydrogel precursor component is provided in the kit, wherein the third hydrogel precursor component comprises at least one agent that promotes growth of the physiologically relevant tissue selected from the group consisting of EGF or an agent that activates EGF receptor signaling, insulin or an insulin receptor agonist or insulin mimetic, and hydrocortisone or an analog or derivative thereof. In some embodiments, the third hydrogel precursor component is provided in the kit, wherein the third hydrogel precursor component comprises at least two agents that promotes growth of the physiologically relevant tissue selected from the group consisting of EGF, insulin and hydrocortisone. In some embodiments, the third hydrogel precursor component is provided in the kit, wherein the third hydrogel precursor component comprises at least two agents that promotes growth of the physiologically relevant tissue selected from the group consisting of EGF or an agent that activates EGF receptor signaling, insulin or an insulin receptor agonist or insulin mimetic, and hydrocortisone or an analog or derivative thereof. In some embodiments, the third hydrogel precursor component is provided in the kit, wherein the third hydrogel precursor component comprises EGF, insulin and hydrocortisone. In some embodiments, the third hydrogel precursor component is provided in the kit, wherein the third hydrogel precursor component comprises EGF or an agent that activates EGF receptor signaling, insulin or an insulin receptor agonist or insulin mimetic, and hydrocortisone or an analog or derivative thereof. In some embodiments, the third hydrogel precursor component is provided in the kit, wherein the third hydrogel precursor component comprises one or more agents that promote growth and/or differentiation of nervous system tissue, e.g., one or more neurotrophins, e.g., NGF, and/or one or more other agents that activate NGF receptor signaling.

In some embodiments, the kit further includes at least one cell. The at least one cell can be provided in a container with the first, second, and/or third hydrogel precursor component, or in a separate container. In some embodiments, the kit includes the first, second, and optionally third hydrogel precursor component and the at least one cell. In some embodiments, the kit includes the first, second and third hydrogel precursor components and the at least one cell. In some embodiments, the kit includes instructions for obtaining at least one cell from a subject for culturing in the three-dimensional hydrogel.

In some embodiments, the kit further includes a culture medium. In some embodiments, the culture medium comprises a defined culture medium. In some embodiments, the culture medium is substantially free of serum. Examples of suitable culture medium include, without limitation, DMEM, Ham's F-12, and combinations thereof. In some embodiments, the culture medium comprises a 50:50 mixture of DMEM and Ham's F-12. In some embodiments, the culture medium comprises a 10:90 mixture, a 20:80 mixture, a 30:70 mixture, a 40:60 mixture, a 50:50 mixture, a 60:40 mixture, a 70:30 mixture, a 80:20 mixture, or a 90:10 mixture of DMEM and Ham's F-12. Other non-limiting examples of suitable culture medium include, without limitation, Opti-MEM™, MEGM, FAD2, and mixtures of any two of the foregoing at any of the afore-mentioned ratios. In some embodiments, the culture medium is free of ROCK inhibitor and/or forskolin. In some embodiments, the culture medium or kit comprises one or more ROCK inhibitor, ALK5 inhibitor, or both. In some embodiments, the culture medium comprises at least one agent, at least two agents, at least three agents, at least four agents, or at least five agents that stimulate development of a physiologically relevant tissue (e.g., mammary tissue) in vivo. In some embodiments, the kit includes instructions for culturing at least one cell in the three-dimensional hydrogel. In some embodiments, the kit includes instructions for adding the culture medium to the three-dimensional hydrogel. In some embodiments, the kit includes instructions for replenishing the culture medium e.g., replenishing the culture medium every other day, every third day, every fourth day, every fifth day, every sixth day, every week, every eighth day, every ninth day, every tenth day. In some embodiments, the kit includes instructions for adding at least one agent, at least two agents, at least three agents, at least four agents, or at least five agents that stimulate development of the physiologically relevant tissue (e.g., mammary tissue) in vivo at day 0, at day 1, at day 2, at day 3, at day 4, at day 5, at day 6, at day 7, at day 8, at day 9, at day 10, at day 11, at day 12, at two weeks, at two and a half weeks, at three weeks, at three weeks, at day 28, at four weeks, at one month, at five weeks, or at six weeks. In some embodiments, the at least one, at least two, at least three, at least four, or at least five agents that stimulate development of the physiologically relevant tissue (e.g., mammary) in vivo are added at to the culture the same day. In some embodiments, the at least one, at least two, at least three, at least four, or at least five agents that stimulate development of the physiologically relevant tissue (e.g., mammary) in vivo are added to the culture on the same day. In some embodiments, the at least one, at least two, at least three, at least four, or at least five agents that stimulate development of the physiologically relevant tissue (e.g., mammary) in vivo are added to the culture on the different days. In some embodiments, a first at least one agent that stimulates development of the physiologically relevant tissue (e.g., mammary) in vivo is added to the culture medium on a first day, a second at least one agent is added to the culture on a second day, and a third at least one agent is added to the culture medium on the same day as the first agent, the same day as the second agent, or on a third day after the first and second agents are added to the culture medium.

Exemplary agents that stimulate development of mammary tissue in vivo include, without limitation, steroid hormones, pituitary hormones, lactogenic hormones, and derivatives and combinations thereof.

In some embodiments, the steroid hormone is selected from the group consisting of estrogen and progesterone. In some embodiments, the estrogen comprises isolated estrogen. In some embodiments, the estrogen comprises a synthetic estrogen. In some embodiments, the estrogen comprises 17-beta estradiol. In some embodiments, the estrogen comprises diethylstilbestrol. In some embodiments, the estrogen comprises beta estradiol. In some embodiments, the estrogen comprises a derivative or analog of estrogen. The estrogen can be added to the culture medium or the three-dimensional hydrogel in various concentrations. The presently disclosed subject matter contemplates adding effective amounts of estrogen to the culture medium or the three-dimensional hydrogel. In some embodiments, the estrogen is present at a concentration of between 1 ng/mL and 100 ng/mL. In some embodiments, the progesterone comprises isolated progesterone. In some embodiments, the progesterone comprises synthetic progesterone. In some embodiments, the progesterone comprises a derivative or analog of progesterone. In some embodiments, the progesterone comprises danazol, norethisterone, medroxyprogesterone acetate, or 17-hydroxyprogesterone caproate. The progesterone can be added to the culture medium or the three-dimensional hydrogel in various concentrations. The presently disclosed subject matter contemplates adding effective amounts of progesterone to the culture medium or the three-dimensional hydrogel. In some embodiments, the progesterone is present in the culture medium or three-dimensional hydrogel at a concentration of between 1 ng/mL and 100 ng/mL.

In some embodiments, the pituitary hormone comprises a hormone or growth factor present in pituitary extract. The presently disclosed subject matter contemplates adding effective amounts of pituitary hormone to the culture medium or the three-dimensional hydrogel.

Exemplary hormones or growth factors present in the pituitary extract include, without limitation, growth hormone, fibroblast growth factor, prolactin and follicle stimulating hormone. In some embodiments, the lactogenic hormone is prolactin. In some embodiments, the prolactin is isolated prolactin. In some embodiments, the prolactin is recombinant prolactin. In some embodiments, the prolactin is recombinant human prolactin.

In some embodiments, the kit includes at least one agent that stimulates development of mammary tissue in vivo selected from the group consisting of estrogen, progesterone, pituitary extract, and prolactin. In some embodiments, the kit includes at least two agents that stimulate development of mammary tissue in vivo selected from the group consisting of estrogen, progesterone, pituitary extract, and prolactin. In some embodiment, the kit includes at least three agents that stimulate development of mammary tissue in vivo selected from the group consisting of estrogen, progesterone, pituitary extract, and prolactin. In some embodiments, the kit includes estrogen, progesterone, pituitary extract, and prolactin.

In some embodiments, the kit includes instructions for adding at least one agent, at least two agents, at least three agents, at least four agents, and/or at least five agents that stimulate development of mammary tissue in vivo to the culture medium or three-dimensional hydrogel for a period of time, e.g., for at least 1 day, at least 2 days, at least 3 days, at least 4 days, at least 5 days, at least 6 days, at least 7 days, at least 10 days, at least 12 days, at least 2 weeks, at least 15 days, at least 18 days, at least 3 weeks, at least 4 weeks, at least 5 weeks, or at least 6 weeks, or more. In some embodiments, the kit includes instructions for adding at least one agent, at least two agents, at least three agents, at least four agents, and/or at least five agents that stimulate development of mammary tissue in vivo to the culture medium or the three-dimensional hydrogel for a period of time sufficient for ducts and lobules to form and hollow in the three-dimensional hydrogels.

In some embodiments, the kit includes instructions for growing physiologically relevant tissue in the three-dimensional hydrogel. In some embodiments, the kit includes instructions for growing physiologically relevant mammary tissue in the three-dimensional hydrogel. In some embodiments, the kit includes instructions for adding at least one agent, at least two agents, at least three agents, at least four agents, and/or at least five agents that stimulate development of mammary tissue in vivo to the culture medium or the three-dimensional hydrogel for a period of time sufficient for physiologically relevant mammary tissue to grow in the three-dimensional hydrogel.

In some embodiments, the kit includes instructions for purification of epithelium. In some embodiments, the kit includes instructions for dissociating epithelium tissue into at least one cell and at least one cluster of cells. In some embodiments, the kit includes instructions for dissociating epithelium tissue into single cells and clusters of cells.

In some embodiments, the kit includes instructions for purification of nervous system tissue. In some embodiments, the kit includes instructions for dissociating nervous system issue into at least one cell and at least one cluster of cells. In some embodiments, the kit includes instructions for dissociating nervous system tissue into single cells and clusters of cells.

### III. Method of Forming Three-Dimensional Hydrogel

Aspects of the presently disclosed subject matter relate to methods of forming three-dimensional hydrogels that support growth of physiologically relevant tissue when at least one cell is cultured in the three-dimensional hydrogel.

The presently disclosed subject matter contemplates forming three-dimensional hydrogels that are capable of supporting growth of physiologically relevant tissue. In general, the presently disclosed three-dimensional hydrogels are formed by selecting a physiologically relevant tissue of interest to be grown in a presently disclosed three-dimensional hydrogel, selecting appropriate hydrogel precursor components for inclusion in the hydrogel precursor composition based at least in part on the in vivo composition of the tissue of interest and optionally agents known to be involved in promoting growth (and/or differentiation) of the tissue of interest in vivo, and then polymerizing the hydrogel precursor composition to form the three-dimensional hydrogel.

It should be appreciated that any hydrogel precursor composition described in Section I or kit described in Section II can be used to form a three-dimensional hydrogel in accordance with the presently disclosed methods. In some embodiments, the method of forming a three-dimensional hydrogel includes determining an elastic modulus of a tissue of interest in vivo to be grown in the three-dimensional hydrogel, and then combining the hydrogel precursor components in appropriate amounts to produce a three-dimensional hydrogel having an elastic modulus that is comparable to an elastic modulus reported for the tissue of interest in vivo. In some embodiments, the three-dimensional hydrogel comprises an elastic modulus that is similar to an elastic modulus of a tissue of interest in vivo.

Accordingly, in some aspects, the presently disclosed subject matter provides a method of forming a three-dimensional hydrogel that supports growth of a physiologically relevant tissue when at least one cell is cultured in the three-dimensional hydrogel, the method comprising: (a) providing a presently disclosed hydrogel precursor composition (e.g., solution) or a presently disclosed kit; and (b) incubating the hydrogel precursor solution at an elevated temperature for a period of time sufficient for the hydrogel precursor composition (e.g., solution) to polymerize and form a three-dimensional hydrogel that supports growth of a physiologically relevant tissue when at least one cell is cultured in the three-dimensional hydrogel.

In some embodiments, a method of forming a three-dimensional hydrogel that supports growth of a physiologically relevant tissue when at least one cell is cultured in the three-dimensional hydrogel comprises: (a) providing a hydrogel precursor solution comprising: (i) a first hydrogel precursor component selected from the group consisting of at least one, at least two, or at least three extracellular matrix proteins selected from the group consisting of collagen, fibronectin and laminin; (ii) a second hydrogel precursor component comprising hyaluronan or a glycosaminoglycan having a water-chelating ability similar to hyaluronan; and (iii) at least one agent, at least two agents, or at least three agents that promote growth of a physiologically relevant tissue selected from the group consisting of EGF, insulin and hydrocortisone, wherein (i), (ii) and (iii) are dissolved in an aqueous medium; (b) adding at least one cell to the aqueous medium; and (c) incubating the hydrogel precursor solution at an elevated temperature for a period of time sufficient to form a three-dimensional hydrogel with the at least one cell embedded therein, wherein the three-dimensional hydrogel supports growth of physiologically relevant tissue from the at least one cell in the three-dimensional hydrogel.

In some embodiments, a method of forming a three-dimensional hydrogel that supports growth of a physiologically relevant tissue when at least one cell is cultured in the three-dimensional hydrogel comprises: (a) providing a hydrogel precursor solution comprising: (i) a first hydrogel precursor component selected from the group consisting of at least one, at least two, or at least three extracellular matrix proteins selected from the group consisting of collagen, fibronectin and laminin; (ii) a second hydrogel precursor component comprising hyaluronan or a glycosaminoglycan having a water-chelating ability similar to hyaluronan; and (iii) at least one neurotrophin, wherein (i), (ii) and (iii) are dissolved in an aqueous medium; (b) adding at least one cell to the aqueous medium; and (c) incubating the hydrogel precursor solution at an elevated temperature for a period of time sufficient to form a three-dimensional hydrogel with the at least one cell embedded therein, wherein the three-dimensional hydrogel supports growth of physiologically relevant tissue from the at least one cell in the three-dimensional hydrogel.

The presently disclosed subject matter contemplates incubating the hydrogel precursor composition (e.g., solution) at any elevated temperature that is sufficient to polymerize the hydrogel precursor composition (e.g., solution), for example, by causing the viscous liquid present in the solution to harden and form a three-dimensional hydrogel. In some embodiments, the elevated temperature is a temperature that is elevated relative to ambient or room temperature. Preferably, the elevated temperature is less than a temperature that perturbs the biological function of cells present in the hydrogel precursor composition or otherwise causes agents present in the hydrogel precursor composition to degrade or become unstable. In some embodiments, the elevated temperature is a temperature of at least 15° C., 16° C., 17° C., 18° C., 19° C., 20° C., 21° C., 22° C., 23° C., 24° C. 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C. In some embodiments, the hydrogel precursor solution is incubated at a temperature of 34° C. In some embodiments, the hydrogel precursor solution is incubated at a temperature of 35° C. In some embodiments, the hydrogel precursor solution is incubated at a temperature of 36° C. In some embodiments, the hydrogel precursor solution is incubated at a temperature of 37° C. In some embodiments, the hydrogel precursor solution is incubated at a temperature of 38° C.

In practice, the hydrogel precursor composition is added in liquid form to a mold that is used to cast the hydrogel into a three-dimensional shape. The presently disclosed subject matter contemplates a variety of shapes and volumes, as long as the resulting three-dimensional hydrogel is not so large that it limits the availability of oxygen and other nutrients, for example, by preventing their diffusion throughout the hydrogel so as to result in limited tissue growth and death. Exemplary shapes include, without limitation, spherical, teardrop shaped, round, rectangular, oblong, and oval shaped. In some embodiments, the shape of the three-dimensional hydrogel is based on a container, a culture plate, or a well that the hydrogel precursor composition (e.g., solution) is placed into prior to polymerization. In some embodiments, a volume of the hydrogel precursor solution used to form the three-dimensional hydrogel is ranges from between about 5 μl to about 50 ml. In some embodiments, the volume of the three-dimensional hydrogel is about 1 μl, about 2 μl, about 3 μl, about 4 μl, about 5 μl, about 6 μl, about 7 μl, about 8 μl, about 9 μl, about 10 μl, about 11 μl, about 12 μl, about 13 μl, about 14 μl, or about 15 μl, or more.

Without wishing to be bound by theory, it is believed that the growth of the physiologically relevant tissue grown in the three-dimensional hydrogels disclosed herein will be limited at least in part by the dimensions of the three-dimensional hydrogel used to grow the tissue. In some embodiments, the dimensions (e.g., size, shape and volume) of the mold used to cast a three-dimensional hydrogel of the presently disclosed subject matter are selected based on the size and morphology of a physiologically relevant tissue of interest.

### IV. Three-Dimensional Hydrogel

Aspects of the presently disclosed subject matter relate to three-dimensional hydrogels that support growth of physiologically relevant tissue when at least one cell is cultured in the three-dimensional hydrogel. In some embodiments, a three-dimensional hydrogel that supports growth of a physiologically relevant tissue when at least one cell is cultured in the three-dimensional hydrogel, the three-dimensional hydrogel consists of, consists essentially of, or comprises: (a) at least one extracellular matrix protein selected from the group consisting of collagen, fibronectin, and laminin; (b) hyaluronan or a glycosaminoglycan having a water-chelating ability that is similar to hyaluronan; (c) at least one agent that promotes growth of a physiologically relevant tissue; and (d) at least one cell, wherein (a) and (b) are polymerized into a three-dimensional hydrogel and (c) and (d) are embedded in the three-dimensional hydrogel; and wherein the three-dimensional hydrogel supports growth of a physiologically relevant tissue when (d) is cultured in the three-dimensional hydrogel in the presence of (c).

In some embodiments, the three-dimensional hydrogel is free of synthetic polymers.

In some embodiments, the three-dimensional hydrogel comprises at least two extracellular matrix proteins selected from the group consisting of collagen, fibronectin, and laminin. In some embodiments, the three-dimensional hydrogel comprises collagen, fibronectin, and laminin.

In some embodiments, the three-dimensional hydrogel comprises at least two agents that promote growth of a physiologically relevant tissue. In some embodiments, the three-dimensional hydrogel comprises at least three agents that promote growth of a physiologically relevant tissue. In some embodiments, the at least one, at least two agents, and/or at least three agents that promote growth of a physiologically relevant tissue are selected from the group consisting of EGF or an agent that activates EGF receptor signaling, insulin or an insulin receptor agonist or insulin mimetic, and hydrocortisone or an analog or derivative thereof. In some embodiments, the at least one, at least two agents, and/or at least three agents that promote growth of a physiologically relevant tissue are selected from the group consisting of NGF, NT-3, NT-4, BDNF, and other NGF receptor agonists.

In some embodiments, the three-dimensional hydrogel comprises at least two extracellular matrix proteins selected from the group consisting of collagen, fibronectin, and laminin and at least two agents that promote growth of a physiologically relevant tissue selected from the group consisting of EGF or an agent that activates EGF receptor signaling, insulin or an insulin receptor agonist or insulin mimetic, and hydrocortisone or an analog or derivative thereof. In some embodiments, the three-dimensional hydrogel comprises collagen, fibronectin, and laminin, and EGF or an agent that activates EGF receptor signaling, insulin or an insulin receptor agonist or insulin mimetic, and hydrocortisone or an analog or derivative thereof. In some embodiments, the three-dimensional hydrogel comprises collagen, fibronectin, and laminin, and NGF, NT-3, NT-4, BDNF, and/or one or more other NGF receptor agonists. In some embodiments, the three-dimensional hydrogel comprises collagen, fibronectin, and laminin, and NGF.

The presently disclosed subject matter contemplates culturing any at least one cell in the three-dimensional hydrogel. It should be appreciated that the at least one cell can be selected for culturing in the three-dimensional hydrogel based at least in part on the physiologically relevant tissue of interest to be grown and/or the types of cells to be expanded in the three-dimensional hydrogel.

Exemplary cells include, without limitation, stem cells, primary cells, transdifferentiated cells, dedifferentiated cells, reprogrammed cells, multipotent cells, and pluripotent cells.

In some embodiments, the at least one cell comprises a single cell. In some embodiments, the at least one cell comprises a single cell selected from the group consisting of a colon cell, a gall bladder cell, an intestine cell, a kidney cell, a liver cell, a lung cell, a mammary cell, an ovarian cell, a cervical cell, a pancreatic cell, a prostate cell, and a stomach cell.

In some embodiments, the at least one cell comprises a neuron. In some embodiments, the at least one cell comprises a ganglion-derived cell. In some embodiments, the at least one cell comprises a neural crest cell. In some embodiments, the at least one cell comprises a melanocyte.

In some embodiments a hydrogel as described herein is used to culture cells of two or more cell types. Such cells may, for example, be present together in a tissue fragment or may be mixed together as individual cells prior to seeding or may be added separately to a hydrogel precursor solution prior to polymerization or may develop from a common precursor within the hydrogel. In some embodiments, for example, a hydrogel is used to culture neurons and glial cells. In some embodiments the neuron is a sensory or motor neuron. In some embodiments the neuron is a sensory or motor neuron and the glial cell is a Schwann cell.

In some embodiments, the at least one cell comprises a cell line, at least one cluster of cells, or at least one tissue fragment. The cell line, cell cluster, or tissue fragments can comprise any number of relevant cells. In some embodiments, the cell line, cell cluster, or tissue fragment comprises at least 2 cells, at least 5 cells, at least 10 cells, at least 25 cells, at least 50 cells, at least 60 cells, at least 70 cells, at least 80 cells, at least 90 cells, at least 100 cells, at least 250 cells, at least 300 cells, at least 400 cells, at least 500 cells, at least 750 cells, at least 1,000 cells, at least 10,000 cells, at least 100,000 cells, or more. In some embodiments, the at least one cluster of cells comprises a cluster of between about 50 and 100 cells. In some embodiments, the at least one cluster of cells comprises a cluster of about 10 cells, about 20 cells, about 30 cells, about 33 cells, about 40 cells, about 50 cells, about 60 cells, about 66 cells, about 70 cells, about 75 cells, about 80 cells, about 88 cells, about 90 cells, or about 99 cells. In some embodiments the at least one cluster of cells comprises a cluster of between 2 and 50 cells, between 50 and 100 cells, between 100 and 1000 cells, between 1000 and 10,000 cells, or between 10,000 and 100,000 cells. In some embodiments the at least one cluster of cells comprises a cluster of between 100,000 and 1 million cells, between 1 million and 10 million, or between 10 million and 100 million cells.

Generally, the at least one cell or at least one cluster of cells can include a marker for visualizing, imaging, and/or staining of the at least one cell or at least one cluster of cells. For example, cells or cell clusters can be labeled e.g., with fluorescent proteins before seeding them into a presently disclosed three-dimensional hydrogel, e.g., via lentiviral vectors at a low multiplicity of infection. In some embodiments, the at least one cluster of cells comprises a detectable label. In some embodiments, the at least one cluster of cells comprises a fluorescent protein. Any suitable fluorescent protein can be used. Examples of fluorescent proteins include, without limitation, mCherry, Venus, and Cerulean fluorescent proteins.

The at least one cell or at least one cluster of cells can be depleted for undesirable components prior to seeding in a presently disclosed three-dimensional hydrogel. In some embodiments, at one cell or at least one cluster of cells is depleted for stromal cells. In some embodiments, at least one cell or at least one cluster of cells is depleted for fibroblasts.

In some embodiments, the at least one cell or at least one cluster of cells comprises epithelial cells. In some embodiments, the epithelial cells are not immortalized by transduction with viral oncogenes. In some embodiments of the presently disclosed solution, kit, hydrogel, physiologically relevant tissue, or method, the epithelial cells are not immortalized by introduction of non-endogenous genetic material. In some embodiments of the presently disclosed solution, kit, hydrogel, physiologically relevant tissue, or method, the epithelial cells are not modified by introduction of non-endogenous genetic material. In some embodiments, the epithelial cells are selected from the group consisting of colon cells, gall bladder cells, intestine cells, kidney cells, liver cells, lung cells, mammary cells, ovarian cells, cervical cells, pancreatic cells, prostate cells and stomach cells. In some embodiments, the epithelial cells comprise mammary epithelial cells. In some embodiments, the epithelial cells comprise a disorganized cluster of mammary epithelial cells comprising intermixed CK14+ basal and CK8/18+ luminal cells. In some embodiments, the at least one cell or at least one cluster of cells comprises cancer cells. In some embodiments, the cancer cells are ER/PR positive breast cancer cells.

In some embodiments the at least one cell or at least one cluster of cells comprises non-epithelial cells. In some embodiments, the non-epithelial cells comprise neural cells, e.g., neurons. In some embodiments the non-epithelial cells are not immortalized by transduction with viral oncogenes. In some embodiments the non-epithelial cells are not immortalized by introduction of non-endogenous genetic material. In some embodiments the non-epithelial cells are not modified by introduction of non-endogenous genetic material.

In some embodiments the at least one cell or at least one cluster of cells comprises melanoma cells,

In some embodiments, the at least one cell or at least one cluster of cells can be obtained from a subject. In some embodiments, the at least one cell or at least one cluster of cells comprise mammary epithelial cells obtained from a subject. In some embodiments, the mammary epithelial cells are obtained from a subject selected from the group consisting of: (i) a subject who underwent, or is about to undergo, a breast reduction mammoplasty; (ii) a subject who underwent, or is about to undergo, a breast reconstruction or breast augmentation surgery; (iii) a subject who has, or is suspected of having, breast cancer; (iv) a subject who has been prescribed, or is taking, an anti-lactogenic medication; (v) a subject for which breastfeeding is contraindicated; (vi) a subject who has, or is suspected of having, lactation failure; and (vii) a subject who has, or is suspected of having, breast hypoplasia, atypical ductal hyperplasia, papillomas, fistulas, inflammation, or other pathological breast conditions. In some embodiments, the breast cancer is selected from the group consisting of ER-positive breast cancer, triple-negative breast cancer, Her2-positive breast cancer, and luminal breast cancer (hormone receptor-positive and -negative). In some embodiments, the breast cancer is ER/PR positive breast cancer. In some embodiments, the subject is a human. In some embodiments, the subject is female.

In some embodiments, the at least one cell or at least one cluster of cells obtained from a subject comprises a tissue of interest obtained from a subject comprising the at least one cell or at least one cluster of cells that are dissociated into single cells, clusters of cells, or tissue fragments. The presently disclosed subject matter contemplates dissociating tissue according to any protocol that is available to the skilled artisan. Those skilled in the art will appreciate that the particular protocol used may depend, for example, on the type of tissue and the extent to which the tissue is to be dissociated (e.g., into single cells, clusters of cells, and/or fragments. In some embodiments, the tissue comprises a tissue of interest that is mechanically dissociated, e.g., using a sterile razor blade. For example, a tissue of interest can be, in some embodiments, dissociated into 3-5 mm³ fragments. In some embodiments, the dissociated tissue (e.g., mechanically) is resuspended in a buffer (e.g., dissociation buffer, e.g., MEGM (Lonza) containing 3 mg/mL collagenase (Roche), 250 units/mL hyaluronidase (Sigma Aldrich), 1× antibacterial-antimycotic (Gibco) at a concentration of 0.2 gm/mL, and incubated under suitable conditions for a period of time (e.g., with rocking at 37° C. overnight). In some embodiments, the tissue of interest is dissociated into 1-3 mm³ fragments. In some embodiments, the tissue of interest is dissociated into 2-4 mm³ fragments. In some embodiments, the tissue of interest is dissociated into 4-6 mm³ fragments. In some embodiments, the tissue of interest is dissociated into 5-7 mm³ fragments. In some embodiments, the tissue of interest is dissociated into 6-8 mm³ fragments. In some embodiments, the tissue of interest is dissociated into 7-9 mm³ fragments. In some embodiments, the tissue of interest is dissociated into 8-10 mm³ fragments. In some embodiments, the tissue of interest is dissociated into single cells. In some embodiments, the tissue of interest is dissociated into single cells. In some embodiments, the tissue of interest is dissociated into at least one cluster of cells comprising at least 2 cells, at least 3 cells, at least 4 cells, or at least 5 cells. In some embodiments, the tissue of interest is dissociated into at least one cluster of cells comprising between 5 cells and 1000 cells. In some embodiments, the tissue of interest is dissociated into at least one cluster of cells comprising between 10 cells and 500 cells. In some embodiments, the tissue of interest is dissociated into at least one cluster of cells comprising between 20 cells and 400 cells. In some embodiments, the tissue of interest is dissociated into at least one cluster of cells comprising between 30 cells and 300 cells. In some embodiments, the tissue of interest is dissociated into at least one cluster of cells comprising between 40 cells and 200 cells. In some embodiments, the tissue of interest is dissociated into at least one cluster of cells comprising between 50 cells and 100 cells.

In some embodiments, the dissociated tissue of interest is allowed to pellet, e.g., by gravity for a period of time (e.g., about 5 minutes). In some embodiments, the tissue of interest is washed for a number of times (e.g., about 1, about 2, about 3, about 4, about 5, or about 6 times) in a washing buffer (e.g., PBS containing 5% FBS (Sigma), in order to remove associated stromal cells. In some embodiments, the dissociated tissue of interest is depleted for fibroblasts, e.g., by plating the tissue in DMEM containing 10% FBS on tissue culture treated dishes for a suitable period of time (e.g., about 30 min, about 45 min, about 60 min, about 75 min, about 90 min, or about 120 min). In some embodiments, the tissues with the fibroblasts removed are washed, e.g., in PBS and resuspended in culture media.

The presently disclosed three-dimensional hydrogels possess mechanical properties, such as bending strength, compression strength, tensile strength, shear strength, bending modulus, compression modulus, elastic modulus, swelling ratio, etc. that make them suitable for various applications. In some embodiments, the mechanical properties of the hydrogels described herein are tunable based on, for example, conditions of formation (e.g., pH, ionic strength, concentration of protein, temperature, etc.) and/or maintained conditions of the hydrogel (e.g., pH, ionic strength, concentration of protein, temperature, etc.).

In some embodiments, the swelling ratio and/or elastic modulus of a three-dimensional hydrogel can be engineered to be comparable to a swelling ratio and/or elastic modulus reported in the literature for a tissue of interest in vivo. As used herein, a “tissue of interest in vivo” refers to a physiologically relevant tissue of interest that is to be grown using a presently disclosed three-dimensional hydrogel, e.g., from at least one cell, e.g., at least one or at least one cluster of cells obtained from a subject, e.g., a human subject). In some embodiments, the three-dimensional hydrogel has a swelling ratio of between 295 and 320. In some embodiments, the three-dimensional hydrogel has a swelling ratio of about 200, 205, 210, 215, 220, 230, 235, 240, 250, 255, 260, 275, 280, 290, 300, 310, 315, 320, 315, 320, 330, 335, 340, or 350 or more. In some embodiments, the three-dimensional hydrogel has an elastic modulus of between 200 Pa and 400 Pa. In some embodiments, the three-dimensional hydrogel has an elastic modulus of about 150, about 160 Pa, about 165 Pa, about 170 Pa, about 175 Pa, about 180 Pa, about 190 Pa, about 205 Pa, about 210 Pa, about 215 Pa, about 220 Pa, about 230 Pa, about 240 Pa, about 245 Pa, about 250 Pa, about 260 Pa, about 275 Pa, about 280 Pa, about 285 Pa, about 290 Pa, about 300 Pa, about 310 Pa, about 325 Pa, about 330 Pa, about 340 Pa, about 345 Pa, about 350 Pa, about 355 Pa, about 360 Pa, about 365 Pa, about 370 Pa, about 380 Pa, about 395 Pa, about 405 Pa, about 410 Pa, about 415 Pa, about 420 Pa, about 430 Pa, about 440 Pa, or about 450 Pa or more.

In some embodiments, the swelling ratio and/or elastic modulus can be engineered to be similar to but distinct (e.g., greater than or less than) from a swelling ratio and/or elastic modulus reported in the literature for a tissue of interest in vivo. In some embodiments, the swelling ratio of the three-dimensional hydrogel is at least 1, at least 2, at least 3, at least 4, at least 5, at least 6, at least 7 at least 8, at least 9, at least 10, or greater than the swelling ratio of a tissue of interest in vivo (e.g., mammary tissue, e.g., human mammary tissue). In some embodiments, the swelling ratio of the three-dimensional hydrogel is at least 1, at least 2, at least 3, at least 4, at least 5, at least 6, at least 7 at least 8, at least 9, at least 10, or less than the swelling ratio of a tissue of interest in vivo (e.g., mammary tissue, e.g., human mammary tissue). In some embodiments, the elastic modulus of the three-dimensional hydrogel is at least 1 Pa, at least 2 Pa, at least 3 Pa, at least 4 Pa, at least 5 Pa, at least 6 Pa, at least 7 Pa at least 8 Pa, at least 9 Pa, at least 10 Pa, or greater than the swelling ratio of a tissue of interest in vivo (e.g., mammary tissue, e.g., human mammary tissue). In some embodiments, the swelling ratio of the three-dimensional hydrogel is at least 1 Pa, at least 2 Pa, at least 3 Pa, at least 4 Pa, at least 5 Pa, at least 6 Pa, at least 7 Pa at least 8 Pa, at least 9 Pa, at least 10 Pa, or less than the swelling ratio of a tissue of interest in vivo (e.g., mammary tissue, e.g., human mammary tissue). In some embodiments, the three-dimensional hydrogel is transparent.

In an exemplary embodiment, a three-dimensional hydrogel that supports growth of a physiologically relevant tissue when at least one cell is cultured in the three-dimensional consists of, consists essentially of, or comprises: (a) extracellular matrix proteins collagen, fibronectin, and laminin; (b) hyaluronan or a glycosaminoglycan having a water-chelating ability that is similar to hyaluronan; (c) EGF, insulin, and hydrocortisone; and(d) at least one cell, wherein (a) and (b) are polymerized into a three-dimensional hydrogel and (c) and (d) are embedded in the three-dimensional hydrogel; and wherein the three-dimensional hydrogel supports growth of a physiologically relevant tissue when (d) is cultured in the three-dimensional hydrogel in the presence of (c).

In an exemplary embodiment, a three-dimensional hydrogel that supports growth of a physiologically relevant mammary tissue when at least one mammary epithelial cell or at least one cluster of mammary epithelial cells is cultured in the three-dimensional consists of, consists essentially of, or comprises: (a) extracellular matrix proteins collagen, fibronectin, and laminin; (b) hyaluronan or a glycosaminoglycan having a water-chelating ability that is similar to hyaluronan; (c) EGF, insulin, and hydrocortisone; and(d) at least one mammary epithelial cell or at least one cluster of mammary epithelial cells (e.g., obtained from a subject, e.g., a human subject), wherein (a) and (b) are polymerized into a three-dimensional hydrogel and (c) and (d) are embedded in the three-dimensional hydrogel; and wherein the three-dimensional hydrogel supports growth of physiologically relevant mammary tissue when (d) is cultured in the three-dimensional hydrogel in the presence of (c).

In an exemplary embodiment, a three-dimensional hydrogel that supports growth of a physiologically relevant tumor tissue when at least one cancerous epithelial cell or at least one cluster of cancerous epithelial cells is cultured in the three-dimensional consists of, consists essentially of, or comprises: (a) extracellular matrix proteins collagen, fibronectin, and laminin; (b) hyaluronan or a glycosaminoglycan having a water-chelating ability that is similar to hyaluronan; (c) EGF, insulin, and hydrocortisone; and (d) at least one cancerous epithelial cell or at least one cluster of cancerous epithelial cells (e.g., obtained from a subject, e.g., a human subject), wherein (a) and (b) are polymerized into a three-dimensional hydrogel and (c) and (d) are embedded in the three-dimensional hydrogel; and wherein the three-dimensional hydrogel supports growth of physiologically relevant tumor tissue when (d) is cultured in the three-dimensional hydrogel in the presence of (c).

### V. Physiologically Relevant Tissue

Aspects of the presently disclosed subject matter relate to methods for growing physiologically relevant tissue from at least one cell, and physiologically relevant tissue or component thereof produced in accordance with the methods.

In some embodiments, a method for growing a physiologically relevant tissue from at least one cell consists of, consists essentially of, or comprises: (a) providing a presently disclosed three-dimensional hydrogel; (b) optionally providing a defined culture medium; and (c) culturing the at least one cell in the three-dimensional hydrogel, in the presence of the defined culture medium if provided, for a period of time sufficient for the at least one cell to grow into a physiologically relevant tissue or physiologically relevant component thereof.

As used herein, a “physiologically relevant tissue” refers to a tissue structure grown in a presently disclosed three-dimensional hydrogel that includes one or more physiologically relevant characteristics. The term “physiologically relevant characteristics” refer to characteristics of tissue systems that are similar both structurally and functionally to those found in in vivo tissues, including human tissues. The methods produce tissue structures with similar cellular organization, morphology, histology to in vivo tissue systems, including expression of cellular marker(s) characteristic of the in vivo tissues. Physiologically relevant characteristics include, but are not limited to, one or more differentiated and functional cells or cell types; production of extracellular matrix components; assembly into relevant three-dimensional aggregates; and physiologically relevant cell type ratios, and expression of cellular marker(s) characteristic of the relevant cell types. Physiologically relevant characteristics can differ depending on the particular tissue system.

In some embodiments, the physiologically relevant tissue comprises epithelium. In some embodiments, the physiologically relevant tissue comprises ductal or glandular epithelium. In some embodiments, the physiologically relevant tissue comprises ductal or glandular epithelium tissue selected from the group consisting of gall bladder, intestine, kidney, liver, lung, mammary, ovary, cervix, pancreas, prostate, and stomach. In some embodiments, the physiologically relevant tissue comprises nervous system tissue. In some embodiments, the physiologically relevant tissue comprises a tumor. In some embodiments, the physiologically relevant tissue comprises at least one cell having a mutation in an oncogene or a tumor suppressor gene. In some embodiments, the mutation comprises a loss of function mutation. In some embodiments, the mutation comprises a gain of function mutation.

In particular embodiments, the physiologically relevant tissue comprises mammary epithelium and at least one mammary epithelial cell or at least one cluster of mammary epithelial cells is cultured in the three-dimensional hydrogel. When at least one mammary epithelial cell, or at least one cluster of mammary epithelial cells, is cultured in the three-dimensional hydrogel, the at least one mammary epithelial cell, or at least one cluster of mammary epithelial cells, grows into physiologically relevant mammary tissue in the three-dimensional hydrogel.

In various embodiments, during growth of the physiologically relevant mammary tissue, the cultured cells and/or growing physiologically relevant mammary tissue exhibits at least one of the following physiologically relevant characteristics: i) ductal initiation and/or ductal elongation; ii) a tip at a leading edge of at least one elongating duct, wherein the tip comprises one or two leader cells polarized in the direction of ductal elongation; iii) leader cells expressing basal cytokeratins, staining positively for filamentous actin, and co-expressing SLUG and SOX9; iv) organization into expanding tissues comprising an outer CK14+ basal layer and interior CK8/18+ luminal cells; v) lobule interiors expressing luminal lineage marker GATA3, and luminal differentiation marker MUCl; vi) cavitation of lobule interiors; vii) secondary and tertiary ductal branching selected from the group consisting of bifurcated elongated ducts and side-branches sprouted from primary ducts; viii) lipid droplets; ix) hormone-responsiveness; x) terminal ductal-lobular units (TDLUs), wherein at least a portion of the cells comprising the TDLUs are SLUG+/SOX9+ mammary stem cells; xi) TDLUs comprising layers of between 5 and 8 cells; xii) expression of hormone receptors selected from the group consisting of estrogen receptors, progesterone receptors, glucocorticoid receptors, and androgen receptors; and expression of cellular marker(s) characteristic of mammary epithelial cells.

Physiologically relevant mammary tissue grown in the presently disclosed three-dimensional hydrogels by culturing at least one mammary epithelial cells or at least one cluster of mammary epithelial cells exhibit, in some embodiments, increased ductal, lobular and ductal-lobular growth compared to mammary epithelial cells cultured in three-dimensional basement membrane scaffolds or three-dimensional collagen scaffolds. In some embodiments, the physiologically relevant mammary tissue exhibits increased ductal branching, increased cross-sectional area, increased cell number (counting nuclei), maintenance of hormone receptor expression (ER and PR), present of multiple cell types and proper orientation of cell types within the tissue structures, and viability of long-term cultures. In some embodiments, in contrast to other systems where viability is only maintained for days, tissue structures grown in the presently disclosed three-dimensional hydrogels maintain viability for at least 6 weeks. In some embodiments, the tissue structures remain viable in the three-dimensional hydrogels for at least 1 week, at least 2 weeks, at least 3 weeks, at least 4 weeks, at least 5 weeks, at least 6 weeks, or more. In some embodiments, the physiologically relevant mammary tissue grown in the three-dimensional hydrogel is viable in the three-dimensional hydrogel for at least six weeks. In some embodiments, the physiologically relevant mammary tissue grown in the three-dimensional hydrogel exhibits ductal-lobular morphologies observed in human breast tissue in vivo. In some embodiments, the mammary tissue structures are produced at a seeding efficiency of at least 5%, at least 10%, at least 15%, or at least 25%. In some embodiments, tissue structures of the physiologically relevant mammary tissue achieve maximum growth and/or expansion after the cells are cultured for at least 2 weeks, at least 3 weeks, at least 4 weeks, at least 5 weeks, or at least 6 weeks. In some embodiments, the tissue structures of the physiologically relevant mammary tissue have a diameter of at least 1 mm, at least 2 mm, or at least 3 mm.

The tissue structures of the physiologically relevant mammary tissue can be passaged by removal of the tissue structures from the three-dimensional hydrogel via enzymatic digestion and reseeding the tissue structures thus removed in fresh hydrogels.

In some embodiments, the physiologically relevant mammary tissue grown in the three-dimensional hydrogel secretes milk. In some embodiments, the milk is human milk.

### VI. Milk Production and Related Methods

Aspects of the presently disclosed subject matter relate to methods for producing hormone-responsive, milk-producing tissue and related methods for producing milk.

In some aspects, a method for producing hormone-responsive, milk-producing mammary tissue comprises culturing at least one mammary epithelial cell or at least one cluster of mammary epithelial cells in a presently disclosed three-dimensional hydrogel in the presence of at least one agent that stimulates development of mammary tissue in vivo for a sufficient amount of time to produce hormone-responsive, milk-producing mammary tissue.

In some aspects, a method for producing milk comprises culturing the hormone-responsive, milk-producing mammary tissue produced according to the methods herein for a sufficient amount of time to produce an amount of milk.

In some aspects, a method for treating a subject in need thereof comprises: (a) obtaining at least one mammary epithelial cell or at least one cluster of mammary epithelial cells; (b) culturing the at least one mammary epithelial cell or the at least one cluster of mammary epithelial cells in a presently disclosed three-dimensional hydrogel according in the presence of at least one agent that stimulates development of mammary tissue in vivo for a sufficient amount of time for outgrowth of the at least one mammary epithelial cell or the at least one cluster of mammary epithelial cells in the three-dimensional hydrogel to occur; and (c) implanting the three-dimensional hydrogel into the subject. In some embodiments, the at least one mammary epithelial cell or at least one cluster of mammary epithelial cells are selected from the group consisting of allogeneic cells and autologous cells. In some embodiments, the subject is in need of breast augmentation or breast reconstruction.

In some embodiments, a method for screening a candidate agent that modulates milk production comprises: (a) administering a candidate agent that modulates milk production to the subject into which the three-dimensional hydrogel was implanted; and (b) measuring milk production in the subject, wherein a change in milk production in said subject as compared to a control identifies said agent as a candidate agent that modulates milk production. In some embodiments, prior to administering the candidate agent to said subject the mammary epithelial cells are allowed to grow for a sufficient amount of time for the mammary tissue to mature and hollow. In some embodiments, said measuring milk production comprises measuring a volume of milk produced prior to and after administering the candidate agent, and wherein said control is the measured volume of milk produced prior to administering the candidate agent.

In some aspects, a method for screening for a candidate agent that modulates milk production comprises: (a) culturing the hormone-responsive, milk-producing mammary tissue produced according to the presently disclosed methods in the presence of a test agent; and (b) measuring an amount of milk produced by the hormone-responsive, milk-producing mammary tissue in the culture in the presence of the test agent as compared to a control amount of milk production, wherein a change in amount of milk produced by the hormone-responsive, milk-producing mammary tissue in the culture in the presence of the test agent as compared to the control amount of milk production indicates that the test agent is a candidate agent that modulates milk production. In some embodiments, the test agent is candidate agent that decreases milk production and a decrease in the amount of milk produced by the hormone-responsive, milk-producing mammary tissue indicates that the test agent is a candidate agent that inhibits milk production. In some embodiments, the test agent is a candidate agent that increases milk production and an increase in the amount of milk produced by the hormone-responsive, milk-producing mammary tissue indicates that the test agent is a candidate agent that increases milk production.

Generally, the amount of milk produced can be determined by any suitable method. In some embodiments, the amount of milk produced is determined by quantifying milk lipids, milk carbohydrates, and/or milk proteins. In some embodiments, lipids are quantified using dyes and stains. In some embodiments, the dyes and stains are selected from the group consisting of oil red o, nile red, and 1,6-Diphenyl-1,3,5-hexatriene. In some embodiments, the lipids are quantified using haematoxylin and eosin staining. In some embodiments, the milk proteins are quantified using antibodies. In some embodiments, the milk proteins are quantified using an analytical technique selected from the group consisting of mass spectrometry, Western blot, enzyme-linked immunosorbent assay (ELISA), immunohistochemistry, and immunofluorescence. In some embodiments, the milk carbohydrates are quantified using colorimetric, mass spectrometric, or fluorescent based assays. In some embodiments, the amount of milk produced is quantified microscopically based on an opacity of the three-dimensional hydrogel cultures.

In some aspects, a method for screening for a candidate agent that modulates milk production and/or lactation comprises: (a) culturing the hormone-responsive, milk-producing mammary tissue produced according to the presently disclosed methods in the presence of a test agent; and (b) measuring expression of one or more milk and/or lactation associated genes in the hormone-responsive, milk-producing mammary tissue in the presence of the test agent as compared to a control, wherein a change in expression of one or more milk and/or lactation associated genes in the hormone-responsive, milk-producing mammary tissue in the presence of the test agent as compared to the control indicates that the test agent is a candidate agent that modulates milk production and/or lactation.

The presently disclosed subject matter contemplates measuring expression of any milk and/or lactation associated gene in the hormone-responsive, milk-producing mammary tissue (or cells comprising the tissue). Exemplary milk and/or lactation associated genes to be measured include, without limitation, LALBA, BCAS, CD36, and SLC5A1, and combinations thereof.

In some embodiments, the test agent is candidate agent that decreases milk production and/or lactation and a decrease in the expression of the one or more milk production and/or lactation associated genes in the hormone-responsive, milk-producing mammary tissue indicates that the test agent is a candidate agent that inhibits milk production. In some embodiments, the test agent is a candidate agent that increases milk production and/or lactation and an increase in the expression of the one or more milk production and/or lactation associated genes in the hormone-responsive, milk-producing mammary tissue indicates that the test agent is a candidate agent that increases milk production. In some embodiments, the test agent increases and/or decreases expression of milk/lactation associated gene LALBA. In some embodiments, the test agent increases and/or decreases expression of milk/lactation associated gene BCAS. In some embodiments, the test agent increases and/or decreases expression of milk/lactation associated gene CD36. In some embodiments, the test agent increases and/or decreases expression of milk/lactation associated gene SLC5A1. In some embodiments, the test agent increases and/or decreases expression of at least two, at least three, or at least four milk/lactation associated genes selected from the group consisting of LALBA, BCAS, CD36, and SLC5A1.

In some embodiments, the method includes comparing the amount of milk produced in the presence of the test agent to an amount of milk produced in the presence of prolactin, wherein an increased amount of milk production in the hormone-responsive, milk-producing tissue in the presence of the test agent relative to the amount of milk production in the hormone-responsive, milk producing tissue in the presence of prolactin indicates that the test agent is a candidate agent that increases milk production.

In some embodiments, the method includes comparing the amount of expression of one or more milk/lactation associated genes in the presence of the test agent to an amount of expression produced in the presence of prolactin, wherein an increased amount of expression in the hormone-responsive, milk-producing tissue in the presence of the test agent relative to the amount of expression in the hormone-responsive, milk producing tissue in the presence of prolactin indicates that the test agent is a candidate agent that increases milk production and/or lactation.

The presently disclosed subject matter contemplates candidate agents that comprise fractionated portions of mixtures to identify active ingredients in the mixtures that modulate milk production. In some embodiments, the mixture comprises an herbal supplement. In some embodiments, the herbal supplement comprises fenugreek.

### VII. Certain Treatment Methods

Aspects of the presently disclosed subject matter relate to methods of treating subjects in need thereof. In some embodiments, a method of treating a subject in need thereof comprises implanting into a subject in need thereof a presently disclosed three dimensional hydrogel, a presently disclosed physiologically relevant tissue or component thereof, or a presently disclosed three-dimensional hydrogel together with the physiologically relevant tissue or component thereof. In some embodiments, the subject is in need of the physiologically relevant tissue or component thereof.

In some embodiments, a method for treating a subject in need of treatment thereof comprises administering to the subject in in need thereof an effective amount of a candidate agent identified in accordance to a presently disclosed method.

In some embodiments, a method for treating a subject in need thereof comprises (a) obtaining at least one mammary epithelial cell or at least one cluster of mammary epithelial cells; (b) culturing at least one mammary epithelial cell or at least one cluster of mammary epithelial cells in a presently disclosed three-dimensional hydrogel optionally in the presence of at least one agent that stimulates development of mammary tissue in vivo for a sufficient amount of time for outgrowth of the at least one mammary epithelial cell or the at least one cluster of mammary epithelial cells in the three-dimensional hydrogel to occur; and (c) implanting the three-dimensional hydrogel into the subject.

In some embodiments, a method for treating a subject in need thereof comprises (a) obtaining at least one neural cell or at least one cluster of neural cells; (b) culturing at least nervous system cell or at least one cluster of nervous system cells in a presently disclosed three-dimensional hydrogel, optionally in the presence of at least one agent that stimulates development of nervous system tissue in vivo for a sufficient amount of time for outgrowth of the at least one nervous system cell or at least one cluster of nervous system cells in the three-dimensional hydrogel to occur; and (c) implanting the three-dimensional hydrogel into the subject. In some embodiments the three-dimensional hydrogel is implanted at a site of nervous system damage. In some embodiments the three-dimensional hydrogel is implanted at a site of nervous system degeneration. In some embodiments, the at least one nervous system cell or at least one cluster of nervous system cells are selected from the group consisting of allogeneic cells and autologous cells.

In some embodiments, a method for personalized treatment of a disease (e.g., cancer) in a patient in need thereof comprises administering to the patient an effective amount of an agent or combination of agents selected in accordance with a presently disclosed screening method.

In some embodiments, the treatment method further comprises monitoring progression of the disease (e.g., cancer). In some embodiments, the method includes selecting a subject for treatment.

In some embodiments, the treatment method comprises administering an effective amount of a conventional cancer treatment to a subject.

In some embodiments the patient is genotyped to determine the presence or absence of one or more mutations or genetic variations known to be associated with a disease to be treated (e.g., cancer). Genotyping may be performed using any method known in the art. In some embodiments genotyping comprises sequencing and/or amplifying at least a portion of one or more human genes, e.g., one or more human genes characterized in that mutations or genetic variations in the gene are known to be associated with the disease. In some embodiments a mutation or genetic variation is one that has been identified in a genome wide association study (GWAS). In some embodiments genotyping comprises exome sequencing, RNA sequencing, or whole genome sequencing. In some embodiments genotyping comprises contacting genomic DNA from a biological sample obtained from the subject with a support, e.g., an array, comprising probes that bind specifically to nucleic acids, e.g., DNA, harboring specific alleles of single nucleotide polymorphisms, mutations, or other genetic variations. In some embodiments cDNA, RNA, mRNA derived from a biological sample obtained from the subject may be used. A probe or primer may comprise a suitable detection reagent and/or a suitable detection reagent may be incorporated during the course of DNA sequencing or amplification. In some embodiments a patient's genome or exome may have been at least partly sequenced and stored (e.g., in a database). The previously determined sequence may be accessed and analyzed to determine whether it harbors one or more genetic variations or mutations in a disease-associated gene or in a genetic modifier associated with a disease (e.g., cancer). Examples of various mutations and genetic variations associated with biological conditions (e.g., diseases, e.g., cancer) are known in the art. A subject may be genotyped with respect to any mutations or genetic variations known in the art that is associated with a disease (e.g., cancer). In some embodiments a therapeutic agent for a subject in need of treatment for a disease (e.g., cancer) may be selected based on the subject's genotype.

### VIII. Certain Screening Methods

Aspects of the presently disclosed subject matter relate to various screening methods (e.g., high throughput screens) for identifying candidate agents.

In some embodiments, a method of screening for a candidate agent that modulates a hormonal response of a hormone-responsive physiologically relevant tissue or component thereof comprises: (a) contacting a hormone-responsive physiologically relevant tissue or component thereof cultured in a presently disclosed three-dimensional hydrogel with a test agent; and (b) assessing a hormonal-response of the hormone-responsive physiologically relevant tissue or component thereof in the presence of the test agent as compared to the hormonal-response of a control hormone-responsive physiologically relevant tissue or component thereof not contacted with the test agent, wherein a change in the hormonal response of the hormone-responsive physiologically relevant tissue or component thereof in the presence of the test agent indicates that the test agent is a candidate agent that modulates the hormonal response of the hormone-responsive physiologically relevant tissue or component thereof.

The presently disclosed subject matter contemplates screening for candidate agents that modulate a hormonal response of any hormone-responsive physiologically relevant tissue or component thereof grown in a presently disclosed three-dimensional hydrogel.

In some embodiments, a method of evaluating the effect of an agent on a biological condition of cells comprises: (a) providing a presently disclosed three-dimensional hydrogel; (b) culturing at least one cell or at least one cluster of cells in the three-dimensional hydrogel for a period of time sufficient for the at least one cell or at least one cluster of cells to expand in the three-dimensional hydrogel; (c) exposing the expanding cells in the three-dimensional hydrogel to a test agent; and (d) evaluating the effect of the test agent on the biological condition of the cells.

In some embodiments, a method of evaluating the effect of an agent on a biological condition of a physiologically relevant tissue comprises: (a) providing a presently disclosed three-dimensional hydrogel; (b) culturing at least one cell or at least one cluster of cells in the three-dimensional hydrogel for a period of time sufficient for a physiologically relevant tissue to grow in the three-dimensional hydrogel; (c) exposing the physiologically relevant tissue growing in the three-dimensional hydrogel to a test agent; and (d) evaluating the effect of the test agent on the biological condition of the physiologically relevant tissue.

The presently disclosed subject matter contemplates evaluating the effect of an agent on any physiologically relevant tissue that can be grown and/or cultured in a presently disclosed three-dimensional hydrogel. In some embodiments, the physiologically relevant tissue comprises epithelium tissue selected from the group consisting of gall bladder, intestine, kidney, liver, lung, mammary, pancreas, prostate, and stomach. In some embodiments, the physiologically relevant tissue comprises non-epithelial tissue, e.g., nervous system tissue.

The presently disclosed subject matter contemplates evaluating the effect of an agent on any cell or cluster of cells that can be grown and/or cultured in a presently disclosed three-dimensional hydrogel. Examples of such at least one cell or at least one cluster of cells include, without limitation, a single cell, a cell line, a stem cell, a primary cell, a transdifferentiated cell, a dedifferentiated cell, a reprogrammed cell, a multipotent cell, and a pluripotent cell. In some embodiments, the at least one cell or at least one cluster of cells is selected from the group consisting of a colon cell, a gall bladder cell, an intestine cell, a kidney cell, a liver cell, a lung cell, a mammary cell, a pancreatic cell, a prostate cell, and a kidney cell. In some embodiments, the at least one cell or at least one cluster of cells is neural crest derived. In some embodiments, the at least one cell comprises a melanocyte. In some embodiments the at least one cell or at least one cluster of cells comprises a neural cell or glial cell. In some embodiments the at least one cell or at least one cluster of cells comprises at least one neuron and at least one glial cell. In some embodiments the neuron is a central nervous system neuron and the glial cell is an oligodendrocyte. In some embodiments the neuron is a peripheral nervous system cell and the glial cell is a Schwann cell. In some embodiments, the at least one cell or at least one cluster of cells comprise cancerous cells, or cells having at least one mutation in an oncogene or a tumor suppressor. In some embodiments, the cancerous cells comprise ER/PR positive breast cancer cells. In some embodiments, the at least one cell or at least one cluster of cells is obtained from a subject. In some embodiments, the subject is a normal healthy subject. In some embodiments, the subject is suffering from a disease, condition, or disorder.

In some aspects, three dimensional culture models described herein may be used for drug development and/or toxicology assays.

The presently disclosed subject matter contemplates evaluating the effect of an agent on cells, tissue, or biological conditions thereof using any method available to the skilled artisan. In some embodiments, evaluating the effect of the agent on the biological condition comprises imaging cells in the three-dimensional hydrogel to determine how the agent affects a phenotype of the cells. In some embodiments, evaluating the effect of the agent on the biological condition identifies at least one of a change in growth rate, cell number, cell shape, viability, function, and morphology of the cells. In some embodiments, evaluating the effect of the agent on the biological condition comprises conducting an omic analysis on the cells selected from the group consisting of genomic analysis, metabolomic analysis, proteomic analysis, and a transcriptomic analysis. In some embodiments, evaluating the effect of the agent on the biological condition comprises conducting an epigenetic analysis on the cells. In some embodiments, evaluating the effect of the agent on the biological condition comprises genotyping cells. At least one cell or at least one cluster of cells can be genotyped before and/or after being cultured in the three-dimensional hydrogel (e.g., after exposure to the agent).

The presently disclosed subject matter contemplates using any agent as a test agent. In some embodiments, the agent is a chemical compound or a biological material. In some embodiments, the agent is electromagnetic radiation, particle radiation, a non-ambient temperature, a non-ambient pressure, acoustic energy, a mechanical force, an electrical field, a magnetic field, and combinations thereof. In some embodiments, the agent is a combination of a chemical compound or a biological material and electromagnetic radiation, particle radiation, a non-ambient temperature, a non-ambient pressure, acoustic energy, a mechanical force, an electrical field, a magnetic field, and combinations thereof.

In some embodiments, the agent is a candidate agent selected from the group consisting of a candidate allergenic agent, a candidate biologic agent, a candidate carcinogenic agent, a candidate estrogenic agent, a candidate immunogenic agent, a candidate lactogenic agent, a candidate mutagenic agent, a candidate nerve agent, a candidate pathogenic agent, a candidate pesticide agent, a candidate radioactive agent, a candidate teratogenic agent, a candidate toxicant agent, and candidate vesicant agent. In some embodiments, the agent (e.g., test agent) is an industrial chemical. In some embodiments, the agent is bisphenol A (BPA) or another industrial chemical suspected of being harmful to human health. In some embodiments, the agent is a candidate therapeutic agent. In some embodiments, the candidate therapeutic agent is a candidate chemotherapeutic agent. In some embodiments, the candidate lactogenic agent comprises prolactin. In some embodiments, the candidate lactogenic agent comprises an agent that mimics prolactin. In some embodiments, the candidate lactogenic agent is an agent that influences milk production and/or lactation comparable to or better than prolactin.

In some embodiments, the biological condition is a biological process. In some embodiments, the biological condition is a biological pathway. In some embodiments, the biological condition is normal unperturbed functioning of a cell, organ or tissue and the agent causes one or more of the cells to become abnormal. In some embodiments, the biological condition is a disease or perturbed functioning of a cell, organ or tissue and the agent causes one or more of the cells to become normal. In some embodiments, the biological condition is selected from the group consisting of cancer, diabetes, a neurodegenerative disease, a cardiovascular disease, and an auto-immune disease. In some embodiments, the biological condition is a cancer. In some embodiments, the biological condition is a cancer, and wherein the cells comprise cancerous epithelial cells from the same tissue or organ. In some embodiments, the cancer is selected from the group consisting of colon cancer, gall bladder cancer, intestine cancer, kidney cancer, liver cancer, lung cancer, mammary cancer, ovarian cancer, cervical cancer, pancreatic cancer, prostate cancer, and stomach cancer. In some embodiments, the cancer is ER/PR positive breast cancer. In some embodiments the cancer is a melanoma. In some embodiments the cancer is a glioma, ganglioglioma, or neuroblastoma.

In some embodiments, a method of screening for a candidate chemotherapeutic agent comprises (a) culturing at least one cancer cell in a presently disclosed three-dimensional hydrogel for a sufficient amount of time for growth of the at least one cancer cell in the three-dimensional hydrogel to occur; (b) exposing the at least one cancer cell in the three-dimensional hydrogel to at least one test agent; and (c) measuring growth of the at least one cancer cell in the three-dimensional hydrogel in the presence of the test agent, wherein a decrease in growth of the at least one cancer cell in the presence of the test agent as compared to a control identifies the agent as a candidate chemotherapeutic agent.

The presently disclosed methods contemplate culturing at least one cancer cell for a sufficient amount of time to expand the at least one cancer cell in the culture by a desired amount. In some embodiments, the at least one cancer cell is cultured for a sufficient amount of time to expand the at least one cancer cell in the culture by at least 2-fold, at least 3-fold, at least 4-fold, at least 5-fold, at least 10-fold, or more. In some embodiments, the at least one cancer cell is cultured for a sufficient amount of time to produce tumor spheroids in the three-dimensional hydrogel. In some embodiments, the at least one cancer cell is cultured for a sufficient amount of time for the at least one cancer cell to exhibit cell invasion in the three-dimensional hydrogel. In some embodiments, the at least one cancer cell is cultured for a period of between about one day, two days, three days, four days, five days, six days, one week, eight days, nine days, 10 days, 11 days, 12 days, 13 days, two weeks, 15 days, 18 days, 21 days, 28 days, or one-month or more.

Generally, the at least one cancer cell is cultured in the three-dimensional hydrogel under conditions suitable for proliferation and/or invasion of the at least one cancer cell to occur. In some embodiments, the at least one cancer cell is cultured in hypoxic oxygen conditions. In some embodiments, the at least one cancer cell is cultured in hypoxic oxygen conditions comprising between 0.1% and 1.0% oxygen.

The at least one cancer cell (or cluster of cancer cells) can be obtained by dissociating tumor tissue obtained from a subject into single cells. In some embodiments, the at least one cancer cell is obtained from an in situ or pre-malignant lesion of the subject. In some embodiments, the subject has, or is suspected of having, breast cancer. In some embodiments, the breast cancer is selected from the group consisting of ER-positive breast cancer, triple-negative breast cancer, Her2-positive breast cancer, and luminal breast cancer (hormone receptor-positive and -negative). In some embodiments, the breast cancer is ER/PR positive breast cancer. In some embodiments, the subject's breast tumor expresses at least one hormone receptor. In some embodiments, the at least one cancer cell retains expression of the at least one hormone receptor in culture in the three-dimensional hydrogel. In some embodiments, the at least one hormone receptor is selected from the group consisting of an epidermal growth factor receptor (EGFR), estrogen receptor, HER2 receptor, a MET receptor, a progesterone receptor, a glucocorticoid receptor, and an androgen receptor. In some embodiments, the subject has, or is suspected of having, melanoma.

In some embodiments, measuring growth of the at least one cancer cell comprises measuring cell proliferation or measuring cell viability of the at least one cancer cell in the three-dimensional hydrogel. In some embodiments, measuring growth of the at least one cancer cell comprises counting surviving cancer cells using microscopy. In some embodiments, the method includes quantifying said surviving cancer cells using dyes and stains that identify living cells. In some embodiments, the method includes quantifying said surviving cancer cells using a plate-reader in combination with dyes and stains that identify living cells. In some embodiments, the method includes quantifying said surviving cancer cells using a plate-reader in combination with a reagent that emits a luminescent signal, a fluorescent signal, or colorimetric signal when contacted with living cells. In some embodiments, the method includes quantifying said surviving cancer cells by barcoding via infection with a pool of retroviruses or lentiviruses, and sequencing DNA to determine the number of said surviving cancer cells. It will be appreciated that any one or more of these methods may be used.

In some embodiments, the method comprises measuring cell death (e.g., apoptosis), DNA damage, invasive phenotype, cell metabolism, secretion, cell mobility, cell differentiation state, and/or genomic, proteomic, epigenomic, and/or transcriptomic changes. In some aspects such changes may serve as indicators of potential anti-cancer activity of the agent. For example, it is contemplated to identify test agents that cause a shift in cell metabolism, secretion, cell mobility, cell differentiation state, and/or genomic, proteomic, epigenomic, and/or transcriptomic properties from a cancer-associated state towards a more normal (non-cancer) state. For example, in some embodiments a decrease in invasive phenotype or an increase in apoptosis in cancer cells exposed to the test agent as compared to a control identifies the agent as a candidate chemotherapeutic agent. In some embodiments a decrease in expression of an oncogene or an increase in expression of a tumor suppressor gene in cancer cells exposed to the test agent as compared to a control identifies the agent as a candidate chemotherapeutic agent.

In some embodiments the method comprises performing a replication labeling assay, a cell membrane integrity assay, a cellular ATP-based viability assay, a mitochondrial reductase activity assay, a caspase activity assay, an Annexin V, a DNA content assay, a DNA degradation assay, or a nuclear fragmentation assay. Exemplary assays include BrdU, EdU, or H3-thymidine incorporation assays; DNA content assays using a nucleic acid dye, such as Hoechst Dye, DAPI, actinomycin D, 7-aminoactinomycin D or propidium iodide; cellular metabolism assays such as AlamarBlue, MTT, XTT, and CellTiter-Glo; nuclear fragmentation assays; cytoplasmic histone associated DNA fragmentation assay; PARP cleavage assay; TUNEL staining; and Annexin staining.

In some embodiments, gene expression analysis (e.g., microarray, cDNA array, quantitative RT-PCR, RNAse protection assay, RNA-Seq) may be used to measure the expression of genes whose products mediate or are correlated with cell cycle, cell survival or cell death (e.g., apoptosis), and/or cell proliferation, as an indication of the effect of an agent on cell viability or proliferation. Alternately or additionally, expression of proteins encoded by such genes may be measured. In some embodiments, cells are modified to comprise an expression vector that includes a regulatory region of a gene whose products mediate or are correlated with cell cycle, cell survival (or cell death), and/or cell proliferation operably linked to a sequence that encodes a reporter gene product (e.g., a luciferase enzyme), wherein expression of the reporter gene is correlated with transcriptional activity of the gene. In such embodiments assessment of reporter gene expression (e.g., luciferase activity) provides an indirect method for assessing cell survival or proliferation. Those of ordinary skill in the art are aware of genes whose products mediate or are correlated with cell cycle, cell survival (or cell death), and/or cell proliferation.

In some embodiments, measuring growth and/or one or more other properties of the at least one cancer cell is performed after exposing the at least one cancer cell in the three-dimensional hydrogel to the candidate chemotherapeutic agent for a period of time. In some embodiments, growth and/or one or more other properties is measured after exposing the at least one cancer cell in the three-dimensional hydrogel to the candidate chemotherapeutic agent for at least one day, at least two days, at least three days, at least four days, at least five days, at least six days, at least seven days, at least eight days, at least nine days, at least 10 days, or more.

In some embodiments, the candidate chemotherapeutic agent is selected from the group consisting of a small organic compound, RNA, DNA, peptide, and an antibody. In some embodiments, the candidate chemotherapeutic agent is selected from the group consisting of RNAi, shRNA, and a genomic editing system. In some embodiments, the genomic editing system is selected from the group consisting of a CRISPR-Cas system, a meganuclease, a zinc finger nuclease, and a transcription activator-like effector-based nuclease (TALEN). In some embodiments, the at least one cancer cell is exposed to multiple test agents in the three-dimensional hydrogel. In some embodiments, the at least one cancer cell is exposed to at least 2, at least 3, at least 4, at least 5, at least 6, at least 7, at least 8, at least 9, at least 10, at least 11, at least 12, at least 15, at least 20, at least 25, at least 33, at least 36, at least 50, or more test agents in the three-dimensional hydrogel. In some embodiments, multiple three-dimensional hydrogels can be used to test multiple different test agents or combinations of agents. In some embodiments, at least 5, 10, 20, 30, 40, 50, 100, 200, 300, 400, 500, or more agents or combinations of agents are tested in multiple individual three-dimensional hydrogels. It should be appreciated that absolute or relative concentrations of agents or combinations of agents could be tested. In some embodiments, at least 2 agents, at least 3 agents, at least 4 agents, or at least 5, or more, agents are selected. In some embodiments, the method includes selecting a combination of agents which when used together results in the greatest decrease in growth of the cancer cells in the three-dimensional hydrogel. In some embodiments, the combination of agents acts synergistically to decrease growth of the cancer cells. In some embodiments, the method comprises selecting an agent or combination of agents that causes a decrease in growth at least as great as that caused by at least 50%, 60%, 70%, 80%, 90% or more of the agents or combinations of agents tested. In some embodiments, the method comprises selecting an agent or combination of agents that causes a decrease in growth by a selected amount relative to growth in the absence of such agent or combination of agents. In some embodiments, the selected amount is at least 50%, 60%, 70%, 80%, 90%, 95%, or more, e.g., 100%. In some embodiments, the method comprises selecting an agent or combination of agents that causes a decrease in growth at least as great as that caused by a reference agent or a reference combination of agents. In some embodiment the reference agent or combination of agents may be agent(s) that are used in a standard, art-accepted treatment regimen for the type of cancer that the patient has. In some embodiments a screen may be performed to identify one or more agents or combinations of agents that, when used in combination with a standard treatment, increases the ability of the standard treatment to decrease growth of the cancer cells.

In some embodiments, a method for personalized treatment of a cancer in a patient in need thereof comprises administering to the patient the agent or combination of agents selected in accordance with the screening method, e.g., wherein the cancer cells cultured in the three-dimensional hydrogel originate from the particular patient's cancer. In some embodiments, the method further includes monitoring growth or survival of cancerous cells in the patient. In some embodiments, a method for personalized treatment of a cancer in a patient in need thereof comprises: (a) providing a sample comprising cells originating from subject in need of treatment of cancer; (b) forming multiple three-dimensional hydrogels each comprising one or more of the cells originating from the subject; (c) contacting the three-dimensional hydrogels with different agents or combinations of agents; and (d) selecting an agent or combination of agents that produces a maximum decrease in growth or a selected decrease in growth. In some embodiments, the method further comprises administering said agent or combination of agents to the subject.

In some aspects, the presently disclosed subject matter provides a method of screening for mechanisms of drug resistance in a patient's own cells. In such aspects, cells (e.g., cancer cells or clusters thereof) can be obtained from a patient, and cultured in a presently disclosed three-dimensional hydrogel. The cultured cells can be exposed to test agents and growth and/or survival of the cells (e.g., rates of change in growth and/or survival of cells) in the three-dimensional hydrogel can be assessed in the presence of the test agent and compared to reference levels of growth and/or survival of cells exposed to the test agent that are not known to exhibit resistance to the test agent. If the rates of change in growth and/or survival of the cells in the presence of the test agent increase or decrease relative to the control, the cells may be exhibiting resistance to the test agent. Once resistance to the test agent is determined, additional tests can be performed to identify the mechanisms by which the cells are resisting the test agent, as will be appreciated by the skilled artisan. In some embodiments, the method includes determining resistance to chemotherapeutic agents. In some embodiments, the method includes methods of determining resistance to antibiotic or antiviral agents and the patient's own cells are cultured in the three-dimensional hydrogels in the presence of a virus or bacteria. and/or evaluating an effect of an agent on a biological condition of a cell or physiologically relevant tissue.

In some aspects, the presently disclosed subject matter comprises a method of screening for a candidate neuromodulatory agent, the method comprising: (a) culturing at least one neural cell (e.g., a neuron) in a presently disclosed three-dimensional hydrogel for a sufficient amount of time for a selected time period; (b) exposing the at least one neural cell in the three-dimensional hydrogel to at least one test agent; and (c) measuring at least one phenotype or activity of the at least one neural cell in the three-dimensional hydrogel in the presence of the test agent, wherein a decrease or increase in the at least one phenotype or activity of the at least one neural cell in the presence of the test agent as compared to a control identifies the agent as a candidate neuromodulatory agent. As used herein, a “neuromodulatory agent” is an agent that modulates the activity, survival, differentiation, and/or one or more phenotypes of a neural cell. In some embodiments the neuron may be of any neuron type. In some embodiments the screening is to identify an agent that inhibits neural activity or an agent that increases neural activity. In some embodiments the screening is to identify an agent that promotes neuron survival and/or differentiation, neurite outgrowth, axon outgrowth, and/or dendrite outgrowth. In some embodiments the screening may determine whether an agent is toxic to neurons, e.g., causes neuron death or degeneration. In some embodiments the cells comprise neurons and glia, and the screening is to identify an agent that promotes myelin production or myelination. In some embodiments the neuron is a peripheral nervous system neuron and the glial cell is a Schwann cell. In some embodiments the neuron is a central nervous system neuron and the glial cell is an oligodendrocyte.

A wide range of neurological disorders may be treated using agents identified using the screening approach described herein, which include any disorders in which neurons are overactive (chronic pain, Parkinson's disease (e.g., tremors associated with Parkinson's disease), tremors associated with other disorders, epilepsy) or underactive (these disorders include various forms of palsy, sclerosis, and paralysis, e.g., motor neuron disorders such as amyotrophic lateral sclerosis, progressive muscular atrophy, spinal muscular atrophy). In some embodiments a neural cell is obtained from a patient suffering from such a disorder or is engineered to harbor a mutation or other genetic abnormality associated with such a disorder. In some embodiments such a neural cell is used in screening to identify a candidate agent for treating the disorder.

In some embodiments, a screen may comprise growing the relevant neurons in the hydrogels and measuring their activity, then applying candidate drug compounds (test agent) to determine whether they increase or decrease the activity of the neurons. This could additionally or alternately be done to measure the effect of a test agent on the response of the neuron to an additional external stimulus, e.g., a physical or chemical stimulus. For instance, heat, pressure, or changes in the salt concentrations of the extracellular medium will cause sensory neurons to fire more rapidly, so one could screen the ability of candidate drugs to inhibit or increase this response to a stimulus. In some embodiments, the stimulus may be application of a neurotransmitter. In some embodiments, the stimulus may be application of a compound that binds to an ion channel, e.g., a transient receptor potential (TRP) channel. In some embodiments, a stimulus may be an electrical stimulus, which may be delivered using an electrode inserted into the hydrogel or placed in contact with the surface of the hydrogel. In some embodiments the stimulus is delivered using a multi-electrode array in contact with the hydrogel, e.g., positioned such that the hydrogel is on top of the array. In some embodiments, neural activity may be measured using an electrode inserted into the hydrogel, either making contact with the neurons, or very close to the neurons. The electrodes can record an electrical impulse up to 200 um away. In some embodiments, neural activity may be measured by setting the entire hydrogel down onto a multi-electrode array and recording from the surface of the hydrogel. In such embodiments it may be desirable that the tissue is growing close to the surface (within 200 um) of the hydrogel. In some embodiments, neural activity may be measured using calcium imaging (see, e.g., Grienberger and Konnerth, Neuron 73: 862-885 (2012)). Chemical fluorescent calcium indicators and/or protein-based genetically encoded calcium indicators may be used. In some aspects, an agent that increases neural activity may be used to treat a disorder associated with abnormally low neural activity. In some aspects, an agent that decreases neural activity may be used to treat a disorder associated with abnormally high neural activity.

In some aspects an agent that increases neuron survival, neuron differentiation, neurite outgrowth, axon or dendrite growth may be used in regenerative medicine applications. For example, such agents may be useful in treatment of traumatic brain injury, spinal cord injury, other injury or damage affecting the central or peripheral nervous system, neurodegenerative diseases, stroke, etc. In some aspects an agent that increases neuron survival, neuron differentiation, neurite outgrowth, axon or dendrite growth may be used in treatment of a disorder associated with neuron loss, such as Parkinson's disease or ALS (associated with loss of dopaminergic neurons and motor neurons, respectively)

In some aspects an agent that increases myelin production may be useful in treatment of disorders associated with demyelination (e.g., disorders in which myelin is damaged or not properly produced), such as multiple sclerosis or Guillain-Barré syndrome.

### IX. Patient Derived Xenografts as Animal Models for Human Disease

Aspects of the presently disclosed subject matter relate to patient-derived xenografts produced using physiologically relevant tissue (e.g., cells, tissues, or organs) cultured in or grown using the three-dimensional hydrogels, or hydrogel precursor solutions thereof, of the presently disclosed subject matter as an animal model for human disease. The term “animal model” as used herein refers to any non-human animals directly or indirectly manipulated (e.g., genetically modified, or grafted with cells or tissue) to include one or more cells bearing altered or exogenous genetic information (e.g., that is exogenous to the animal). In a particular aspect of this invention, the animal model is an immuno-compromised non-human animal capable of receiving and supporting a xenograft without mounting a graft-rejection immune response. An “immuno-compromised” animal can either be an immuno-deficient animal which is genetically deprived of endogenous T cells, B cells, NK cells or a combination thereof. Alternatively or additionally, an animal can be immuno-suppressed by biological or chemical means. Such biological or chemical means include, without limitation, immuno-suppression by repeated treatment with irradiation, cyclosporine, anti-Asialo GM1 antibody, or other immuno-suppressive agents or treatments well known in the art. In some embodiments, the animal models herein emulate or mimic a human disease, for example, proliferative diseases which involve uncontrolled cell growth. In some embodiments, the human disease is a tumor or cancer.

In some embodiments, the human disease is associated with a mutation of a target gene, such as a tumor suppressor gene or oncogene. “Mutation” as used herein includes substitution, deletion, and/or insertion of one or more nucleotides. “Target gene” as used herein refers to a gene of interest. Examples of target genes include, without limitation, PI3K, EGFR, p53, RAS e.g., N-RAS, K-RAS, B-RAF, C-KIT, PDGFRA, BCR-ABL, JAK2K, BRCA1, BRCA2, HER2, and MET. In certain embodiments, mutations of these target genes are associated with a tumor or cancer. In some embodiments, the mutations comprise a gain of function mutation. In some embodiments, the mutations comprise a loss of function mutation.

The term “xenograft” as used herein refers to a graft of tissue or cells taken from a donor which is a species different from the animal model, and grafted into the animal model. In some embodiments, the donor of the xenograft is human. In some embodiments, the xenograft tissue or cells are tumor tissue or cells, or cancerous tissue or cells. In some embodiments, the xenograft is pre-treated before grafting into the animal model. The term “pre-treated” when refers to tissue, generally relates to any processing methods known in the art to treat a tissue before its engraftment, such as washing, homogenization, re-suspension and mixing with a solution (e.g., saline, PBS etc.). The term “pre-treated” when refers to cells, includes any processing methods known in the art to treat cells before its engraftment, such as culture, sub-culture, activating, treatment with an agent, centrifugation, re-suspension, filtration, and mixing with a solution (e.g., saline, PBS etc.). After grafted with xenograft, the animal model is allowed sufficient time to develop a lesion of the human disease for further use.

Generally, the animal model described herein can be used to test or select candidate drug (or combination of candidate drugs) for efficacy on disease development and progression, or to test the efficacy of a conventional drug for a disease in the treatment of individuals (e.g., with specific mutations of gene). In some embodiments, the test or selection are carried out in samples or specimens (e.g., blood, a biopsy) from the animals. In some embodiments, the test or selection are carried out by observing the physical changes (e.g. weight loss/gain, size of disease related lesion, decrease in growth and/or survival of cancerous cells) of the animal and/or the xenograft, or by detecting presence or level of a biomarker of interest in the body fluid (e.g. blood) of the animal. In some embodiments, the patient derived xenograft tumor models disclosed herein are useful for preclinical testing of novel anticancer compounds (as well as novel therapeutic and/or synergistic combinations of anticancer compounds) in vivo due to the preservation of key features, which includes invasiveness, stromal reaction, tumor vasculature and cellular diversity of human carcinomas.

In some embodiments, the patient-derived xenograft tumors disclosed herein are established from the transplantation of tumor tissue from a cancer patient cultured and/or expanded in a three-dimensional hydrogel of the presently disclosed subject matter and then transplanted into an immunodeficient animal.

Accordingly, in some aspects, the presently disclosed subject matter provides an immunodeficient animal comprising at least one cancer cell or at least one cluster of cancer cells cultured in a three-dimensional hydrogel of the presently disclosed subject matter, or a hydrogel precursor solution thereof, implanted into it.

The at least one cancer cell or at least one cluster of cancer cells can be obtained from a cancer patient (e.g., via surgical resection) and then dissociated into single cells or clusters. The single cells or clusters can then be cultured in a three-dimensional hydrogel disclosed herein, or added to a hydrogel precursor solution thereof. In some embodiments the hydrogel precursor solution containing the at least one cancer cell or at least one cluster of cancer cells can be injected in aqueous form and allowed to polymerize in the immunocompromised animal. In some embodiments, the hydrogel precursor solution containing the at least one cancer cell or at least one cluster of cancer cells can be polymerized to form a three-dimensional hydrogel before implantation into the immunocompromised animal.

The at least one cancer cell or at least one cluster of cancer cells can be cultured in the three-dimensional hydrogel for a period of time before implantation of the three-dimensional hydrogel (or hydrogel precursor solution thereof) into the immunocompromised animal to form the patient tumor xenograft. In some embodiments, the at least one cancer cell or at least one cluster of cancer cells is cultured in the three-dimensional hydrogel for at least one minute, at least 10 minutes, at least 20 minutes, at least 30 minutes, at least 45 minutes, at least 60 minutes, at least 2 hours, at least 3 hours, at least 4 hours, at least 6 hours, at least 8 hours, at least 10 hours, at least 12 hours, at least 15 hours, at least 18 hours, at least 20 hours, at least 24 hours, at least 30 hours, at least 36 hours, at least 40 hours, at least 42 hours, at least 48 hours, at least 60 hours, at least 3 days, at least 4 days, at least 5 days, at least 6 days, at least 7 days, at least 8 days, at least 9 days, at least 10 days, at least 12 days, at least 15 days, at least 18 days, at least 20 days, at least 21 days, at least 25 days, at least 28 days, at least 30 days, or at least one month or more, before implanting the hydrogel into the animal. In some embodiments, the at least one cancer cell or at least one cluster of cancer cells is cultured in the three-dimensional hydrogel for at least 5 weeks, at least 6 weeks, at least 7 weeks, or at least 8 weeks before implanting the hydrogel into the animal.

Patient-derived xenografts disclosed herein can be made from any immunocompromised animal. In some embodiments, the animal is a mammal. In some embodiments, the mammal is a rodent. In some embodiments, the rodent is a mouse. In some embodiments, the mouse comprises an immunocompromised strain selected from the group consisting of nude, Rag, NOD/SCID or gamma2-null. In some embodiments, the rodent is a rat. In some embodiments, the rodent is a guinea pig. In some embodiments, the rodent is a hamster.

The patient-derived xenograft comprising three-dimensional hydrogel, or hydrogel precursor solution thereof, can be transplanted into the animal in any suitable location. In some embodiments, the three-dimensional hydrogel, or hydrogel precursor solution thereof, is implanted into the mammary gland of the immunocompromised animal. In some embodiments, the three-dimensional hydrogel, or hydrogel precursor solution thereof, is implanted subcutaneously into the immunocompromised animal. In some embodiments, the three-dimensional hydrogel, or hydrogel precursor solution thereof, is implanted intraperitoneally into the immunocompromised animal. In some embodiments, the three-dimensional hydrogel, or hydrogel precursor solution thereof, is transplanted under the kidney capsule of the immunocompromised animal. In some embodiments, the three-dimensional hydrogel, or hydrogel precursor solution thereof, is implanted into a tissue or organ of the same type as that from which the cells originated.

The presently disclosed subject matter contemplates using any type of cancer cell for the at least one cancer cell or at least one cluster of cancer cells. In some embodiments, the at least one cancer cell or at least one cluster of cancer cells comprises epithelial cancer cells. In some embodiments, the at least one cancer cell or at least one cluster of cancer cells comprise epithelial cells obtained from a patient suffering from a cancer of epithelial origin. In some embodiments, the at least one cancer cell or at least one cluster of cancer cells comprise cancerous epithelial cells obtained from a patient suffering from colon cancer, gall bladder cancer, intestine cancer, kidney cancer, liver cancer, lung cancer, mammary cancer, ovarian cancer, cervical cancer, pancreatic cancer, prostate cancer and stomach cancer. In some embodiments, the at least one cancer cell or at least one cluster of cancer cells comprise cells obtained from a patient suffering from a cancer of non-epithelial origin. In some embodiments the at least one cancer cell or at least one cluster of cancer cells comprise cells obtained from a patient suffering from a melanoma, a cancer of the peripheral nervous system, or a cancer of the central nervous system. In some embodiments the tumor of the central or peripheral nervous system is a glioma, ganglioglioma, or neuroblastoma. In some embodiments a glioma is an astrocytoma. In some embodiments a glioma is glioblastoma multiforme (a malignant astrocytoma). In some embodiments, the at least one cancer cell or at least one cluster of cancer cells comprises cells harboring a mutation, such as a mutation in an oncogene or tumor suppressor gene.

In some aspects, the presently disclosed subject matter provides a method of screening for a personalized candidate chemotherapeutic agent or candidate chemotherapeutic treatment regimen for a patient in need thereof, the method comprising: (a) administering a test chemotherapeutic agent or a combination of test chemotherapeutic agents to an immunocompromised animal comprising at least one cancer cell or at least one cluster of cancer cells from the patient cultured in a presently disclosed three-dimensional hydrogel (or hydrogel precursor solution thereof) implanted into the immunocompromised animal; (b) measuring growth and/or survival of cancer cells in the immunocompromised animal; and (c) selecting the test chemotherapeutic agent or the combination of test chemotherapeutic agents resulting in the greatest decrease in growth and/or survival of the cancer cells in the immunocompromised animal or in a selected decrease in growth and/or survival of the cancer cells as a personalized candidate chemotherapeutic agent or candidate chemotherapeutic treatment regimen for the patient.

In some embodiments, the method of screening for a personalized candidate chemotherapeutic agent or candidate chemotherapeutic treatment regimen for a patient includes a step of measuring survival of the immunocompromised animal after administration of the test chemotherapeutic agent or combination of test chemotherapeutic agents as compared to a control survival measurement. In such embodiments, the method further includes a step of selecting the test chemotherapeutic agent or combination of test chemotherapeutic agents resulting in the greatest increase in survival of the immunocompromised animal as compared to the control. In some embodiments, median survival is measured. In some embodiments, overall survival is measured. In some embodiments, disease-free survival is measured. In some embodiments, event-free survival is measured. In some embodiments, progression-free survival is measured.

In some aspects, the presently disclosed subject matter provides a method for the personalized treatment of a cancer patient in need of such treatment, the method comprising administering the personalized candidate chemotherapeutic agent or candidate chemotherapeutic treatment regimen selected in step (c) to the patient. In some embodiments, the method includes administering a conventional cancer treatment selected from the group consisting of surgery, radiation therapy, photodynamic therapy, proton therapy, and combinations thereof.

A “chemotherapeutic agent” is used to connote a compound or composition that is administered in the treatment of cancer. Chemotherapeutic agents include, but are not limited to, alkylating agents, such as thiotepa and cyclophosphamide; alkyl sulfonates, such as busulfan, improsulfan and piposulfan; aziridines, such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas, such as carmustine, chlorozotocin, fotemustine, lomustine, nimustine, ranimustine; antibiotics, such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, such as methotrexate and 5-fluorouracil (5-FU); folic acid analogues, such as denopterin, methotrexate, pteropterin, trimetrexate; purine analogs, such as fludarabine, 6-mercaptopurine, thiamiprine, thioguanine; pyrimidine analogs, such as ancitabine, azacitidine, 6-azauridine, carmofur, cytosine arabinoside, dideoxyuridine, doxifluridine, enocitabine, floxuridine, 5-FU; androgens, such as calusterone, dromostanolone propionate, epitiostanol, mepitiostane, testolactone; anti-adrenals, such as aminoglutethimide, mitotane, trilostane; folic acid replenishers, such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g., paclitaxel and docetaxel; chlorambucil; gemcitabine; 6-thioguanine; mercaptopurine; platinum analogs, such as cisplatin and carboplatin; vinblastine; platinum; etoposide; ifosfamide; mitomycin C; mitoxantrone; vincristine; vinorelbine; navelbine; novantrone; teniposide; daunomycin; aminopterin; xeloda; ibandronate; CPT11; topoisomerase inhibitor RFS 2000; difluoromethylornithine; retinoic acid; esperamicins; capecitabine; and pharmaceutically acceptable salts, acids or derivatives of any of the above.

Chemotherapeutic agents also include anti-hormonal agents that act to regulate or inhibit hormone action on tumors, such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens, such as flutamide, nilutamide, bicalutamide, leuprolide, and goserelin; and pharmaceutically acceptable salts, acids or derivatives of any of the above.

In some embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide, and irinotecan, as well as pharmaceutically acceptable salts, acids, or derivatives of any of these.

In some embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain alternative embodiments, the antimitotic agent comprises a vinca alkaloid, such as vincristine, vinblastine, vinorelbine, or vindesine, or pharmaceutically acceptable salts, acids, or derivatives thereof.

The term “test agent” as used herein refers to any substance, molecule, element, compound, or a combination thereof used for treating the disease. The term “test agent” is intended to include both known therapeutic agents and potential therapeutic agents. A test agent can be in any form including, but not limited to, protein, polypeptide, polynucleotide, small organic/inorganic molecule and the like. A test agent can be a natural product, extracts of a natural product, a synthetic compound or a combination of two or more substances. In some embodiments, the test agent includes antisense compounds. In other embodiments, the test agent includes antibodies. In some embodiments, the test agent is an anti-cancer agent. A “test chemotherapeutic agent” is an agent that is being evaluated for its safety, potency, and/or efficacy in the treatment of cancer. Similarly, a “combination of test chemotherapeutic agents” means any combination of two or more of those agents. In some embodiments, the combination of test chemotherapeutic agents comprises two, three, four, or five or more test chemotherapeutic agents.

In some embodiments, the test chemotherapeutic agent or combination thereof is a known chemotherapeutic agent. In some embodiments, the test chemotherapeutic agent or combination thereof is a chemotherapeutic agent approved for use as such by a regulatory authority (e.g., FDA or EMA). In some embodiments, the test chemotherapeutic agent or combination thereof is an agent that that has not previously been shown to be useful as a chemotherapeutic agent, but has been approved for use by a regulatory authority for a non-cancer indication (e.g., antibiotic agent, antidiabetic agent, antihypertensive agent, anti-inflammatory agent, etc.).

In some embodiments, the test chemotherapeutic agent or combination thereof is an immunotherapeutic agent. As used herein, the term “immunotherapeutic agent” refers to a molecule that can aid in the treatment of a disease by inducing, enhancing, or suppressing an immune response in a cell, tissue, organ or subject. Examples of immunotherapeutic agents include, but are not limited to, immune checkpoint molecules (e.g., antibodies to immune checkpoint proteins), interleukins (e.g., IL-2, IL-7, IL-12, IL-15), cytokines (e.g., interferons, G-CSF, imiquimod), chemokines (e.g., CCL3, CCL26, CXCL7), vaccines (e.g., peptide vaccines, dendritic cell (DC) vaccines, EGFRvIII vaccines, mesothelin vaccine, G-VAX, listeria vaccines), and adoptive T cell therapy including chimeric antigen receptor T cells (CAR T cells).

In some embodiment, the test chemotherapeutic agent or combination thereof is a radiotherapeutic agent. As used herein, a “radiotherapeutic agent” refers to those agents conventionally adopted in the therapeutic field of cancer treatment and includes photons having enough energy for chemical bond ionization such as, for instance, alpha (α), beta (β), and gamma (γ) rays from radioactive nuclei as well as x-rays. The radiation may be high-LET (linear energy transfer) or low-LET. LET is the energy transferred per unit length of the distance. High LET is said to be densely ionizing radiation and Low LET is said to be sparsely ionizing radiation. Representative examples of high-LET are neutrons and alpha particles. Representative examples of low-LET are x-ray and gamma rays. Low LET radiation including both x-rays and γ-rays is most commonly used for radiotherapy of cancer patients. The radiation may be used for external radiation therapy that is usually given on an outpatient basis or for internal radiation therapy that uses radiation that is placed very close to or inside the tumor. In case of internal radiation therapy, the radiation source is usually sealed in a small holder called an implant. Implants may be in the form of thin wires, plastic tubes called catheters, ribbons, capsules, or seeds. The implant is put directly into the body. Internal radiation therapy may require a hospital stay. The ionizing radiation source is provided as a unit dose of radiation and is preferably an x-ray tube since it provides many advantages, such as convenient adjustable dosing where the source may be easily turned on and off, minimal disposal problems, and the like. A unit dose of radiation is generally measured in gray (Gy). The ionizing radiation source may also comprise a radioisotope, such as a solid radioisotopic source (e.g., wire, strip, pellet, seed, bead, or the like), or a liquid radioisotopic filled balloon. In the latter case, the balloon has been specially configured to prevent leakage of the radioisotopic material from the balloon into the body lumen or blood stream. Still further, the ionizing radiation source may comprise a receptacle in the catheter body for receiving radioisotopic materials like pellets or liquids. The radioisotopic material may be selected to emit α, β and γ. Usually, α and β radiations are preferred since they may be quickly absorbed by the surrounding tissue and will not penetrate substantially beyond the wall of the body lumen being treated. Accordingly, incidental irradiation of the heart and other organs adjacent to the treatment region can be substantially eliminated. The total number of units provided will be an amount determined to be therapeutically effective by one skilled in treatment using ionizing radiation. This amount will vary with the subject and the type of malignancy or neoplasm being treated. The amount may vary but a patient may receive a dosage of about 30-75 Gy over several weeks.

Radiotherapeutic agents include factors that cause DNA damage, such as γ-rays, X-rays, and/or the directed delivery of radioisotopes to tumor cells. Other forms of DNA damaging factors are also contemplated such as microwaves and UV-irradiation. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the target cell. In some embodiments, the radiotherapeutic agent is selected from the group consisting of 47Sc, 67Cu, 90Y, 109Pd, 1231, 1251, 1311, 186Re, 188Re, 199Au, 211At, 212Pb, 212B, 32P and 33P, 71Ge, 77As, 103Pb, 105Rh, 111Ag, 119Sb, 121Sn, 131Cs, 143Pr, 161Tb, 177Lu, 1910s, 193MPt, 197H, 43K, 52Fe, 57Co, 67Cu, 67Ga, 68Ga, 77Br, 81Rb/.81MKr, 87MSr, 99MTc, 111In, 113MIn, 127Cs, 129Cs, 1321, 197Hg, 203Pb and 206Bi, as described in U.S. Pat. No. 8,946,168, the entirety of which is incorporated herein by reference.

In some embodiments, the test chemotherapeutic agent or combination thereof comprises an anti-inflammatory agent. As used herein, “anti-inflammatory agent” refers to an agent that may be used to prevent or reduce an inflammatory response or inflammation in a cell, tissue, organ, or subject. Exemplary anti-inflammatory agents contemplated for use include, without limitation, steroidal anti-inflammatory agents, a nonsteroidal anti-inflammatory agent, or a combination thereof. In some embodiments, anti-inflammatory agents include clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone, dexamethasone acetate, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecrolimus, prodrugs thereof, co-drugs thereof, and combinations thereof. The anti-inflammatory agent may also be a biological inhibitor of proinflammatory signaling molecules including antibodies to such biological inflammatory signaling molecules.

Radiation methods suitable for use with the presently disclosed methods include, but are not limited to, stereotactic radiosurgery, fractionated stereotactic radiosurgery, and intensity-modulated radiation therapy (IMRT). It will be understood by those of ordinary skill in the art that stereotactic radiosurgery involves the precise delivery of radiation to a tumorous tissue, for example, a mammary tumor, while avoiding the surrounding non-tumorous, normal tissue.

Because stereotactic radiosurgery is so precise, it allows a higher dose of radiation to be given with more sparing of normal tissue than can be achieved with conventional radiotherapy techniques. To achieve this precision, specific procedures for identifying the position of the tumorous tissue are used. For example, information from magnetic resonance imaging (MRI) and/or computed tomography (CT) scans can be transferred directly to a treatment-planning computer system to create a three-dimensional (3-D) model of the tumor and surrounding normal tissue. The 3-D image allows the position of the abnormality to be treated to be identified and targeted. A complex radiation delivery planning system is used to target a high dose of radiation at the tumor while greatly limiting the dose to nearby normal tissue. Special devices are used to keep the subject still so that the radiation will be aimed with great accuracy at the targeted tumor.

Because of noninvasive fixation devices, stereotactic radiation need not be delivered in a single treatment. The treatment plan can be reliably duplicated day-to-day, thereby allowing multiple fractionated doses of radiation to be delivered. When used to treat a tumor over time, the radiosurgery is referred to as “fractionated stereotactic radiosurgery” or FSR. In contrast, stereotactic radiosurgery refers to a one-session treatment.

The main advantage of fractionation is that it allows higher doses to be delivered to tumorous tissue because of an increased tolerance of the surrounding normal tissue to these smaller fractionated doses. Accordingly, while single-dose stereotactic radiation takes advantage of the pattern of radiation given, fractionated stereotactic radiation takes advantage of not only the pattern of radiation, but also of the differing radiosensitivities of normal and surrounding tumorous tissues. Another advantage of fractionated stereotactic radiation is so-called “iterative” treatment, in which the shape and intensity of the treatment plan can be modified during the course of therapy.

Fractionated stereotactic radiosurgery can result in a high therapeutic ratio, i.e., a high rate of killing of tumor cells and a low effect on normal tissue. The tumor and the normal tissue respond differently to high single doses of radiation vs. multiple smaller doses of radiation. Single large doses of radiation can kill more normal tissue than several smaller doses of radiation can. Accordingly, multiple smaller doses of radiation can kill more tumor cells while sparing normal tissue. In some embodiments, multiple smaller doses are administered every day over weeks, such as for 1, 2, 3, 4, 5, 6, 7 or more weeks. In some embodiments, multiple smaller doses are administered several times a day, several times a week, weekly, bimonthly, or monthly, for example. In some embodiments, the frequency of administration of the fractionated radiotherapy varies depending on the size of the tumor, the location of the tumor, the aggressiveness of the tumor, the intensity of the radiation, and the like.

Another advance in stereotactic radiation treatment is the development of three-dimensional images of the tumor and surrounding tissues. Sophisticated software can take small, e.g., 2-mm, cuts from either CT or MRI scans and converts them into three-dimensional images. Three-dimensional treatment planning delivers a high-precision dose to the tumor, while sparing normal tissue, and can achieve more efficacious results than can be achieved with two-dimensional planning.

It will be understood by those of ordinary skill in the art that stereotactic radiosurgery can be characterized by the source of radiation used, including particle beam (proton), cobalt-60, and linear accelerator (x-ray). A linear accelerator produces high-energy X-ray radiation and is capable of delivering precise and accurate doses of radiation required for radiosurgery. Radiosurgery using a linear accelerator is typically carried out in multi-session, smaller dose treatments so that healthy surrounding tissue is not damaged from too high a dose of radiation. Radiosurgery using linear accelerator technology also is able to target larger brain cancers with less damage to healthy tissues.

As used with the presently disclosed methods provided herein, a “gamma knife” uses multiple, e.g., 192 or 201, highly-focused x-ray beams to make up the “knife” that cuts through diseased tissue. The gamma knife uses precisely targeted beams of radiation that converge on a single point to painlessly “cut” through brain tumors. A gamma knife makes it possible to reach the deepest recesses of the brain and correct disorders not treatable with conventional surgery.

As used with the presently disclosed methods, use of proton beam radiation offers certain theoretical advantages over other modalities of stereotactic radiosurgery (e.g., gamma knife and linear accelerators), because it makes use of the quantum wave properties of protons to reduce doses of radiation to surrounding tissue beyond the target tissue. In practice, the proton beam radiation offers advantages for treating unusually shaped brain tumors. The homogeneous doses of radiation delivered by a proton beam source also make fractionated therapy possible. Proton beam radiosurgery also has the ability to treat tumors outside of the cranial cavity. These properties make proton beam radiosurgery efficacious for post-resection therapy for many chordomas and certain chondrosarchomas of the spine and skull base.

In some embodiments, intensity-modulated radiation therapy (IMRT) can be used. IMRT is an advanced mode of high-precision three-dimensional conformal radiation therapy (3DCRT), which uses computer-controlled linear accelerators to deliver precise radiation doses to a malignant tumor or specific areas within the tumor. In 3DCRT, the profile of each radiation beam is shaped to fit the profile of the target from a beam's eye view (BEV) using a multileaf collimator (MLC), thereby producing a number of beams. More particularly, IMRT allows the radiation dose to conform more precisely to the three-dimensional (3-D) shape of the tumor by modulating the intensity of the radiation beam in multiple small volumes. Accordingly, IMRT allows higher radiation doses to be focused to regions within the tumor while minimizing the dose to surrounding normal critical structures. IMRT improves the ability to conform the treatment volume to concave tumor shapes, for example, when the tumor is wrapped around a vulnerable structure, such as the spinal cord.

Treatment with IMRT is planned by using 3-D computed tomography (CT) or magnetic resonance (MM) images of the patient in conjunction with computerized dose calculations to determine the dose intensity pattern that will best conform to the tumor shape. Typically, combinations of multiple intensity-modulated fields coming from different beam directions produce a custom tailored radiation dose that maximizes tumor dose while also minimizing the dose to adjacent normal tissues. Because the ratio of normal tissue dose to tumor dose is reduced to a minimum with the IMRT approach, higher and more effective radiation doses can safely be delivered to tumors with fewer side effects compared with conventional radiotherapy techniques. IMRT typically is used to treat cancers of the prostate, head and neck, and central nervous system.

In some embodiments, the dosage of radiation applied can vary. In some embodiments, the dosage can range from 1 Gy to about 30 Gy, and can encompass intermediate ranges including, for example, from 1 to 5, 10, 15, 20, 25, up to 30 Gy in dose. In some embodiments, the dosage of radiotherapy is about 8 Gy to about 16 Gy.

As used herein, “photodynamic therapy”, also known as photoradiation therapy, phototherapy, and photochemotherapy, refers to a treatment that uses photosensitizing agents in combination with light to kill cancer cells. The photosensitizing agents kill cancer cells upon light activation.

As used herein, “proton therapy”, also known as proton beam therapy, refers to a treatment that uses a beam of protons to irradiate and kill cancer cells.

In some embodiments, the methods are computer-aided.

In some aspects, the invention provides methods for the identification of druggable targets for drug discovery for treating biological conditions (e.g., cancer or a neurodegenerative disease) and the matching of those targets with particular patient populations who are likely to benefit from compounds that modulate those targets. In some embodiments targets are identified in chemical screens using patient-derived cells cultured in the presently disclosed three-dimensional hydrogels. In some embodiments, targets are identified in chemical screens using a presently disclosed patient-derived xenograft as an animal model for human disease. In some embodiments the relevance of the targets is confirmed by testing the effect of compounds, e.g., small molecules, identified in patient-derived cells (e.g., human cells obtained from patients suffering from a disease (e.g., cancer or a neurodegenerative disease)) that are cultured in a presently disclosed three-dimensional hydrogel and serve as models for a patient-specific diseases and confirming that the compound modulates a phenotype associated with the disease. In some embodiments the relevance of the targets is confirmed by testing the effect of compounds, e.g., small molecules, identified in vivo using the patient-derived xenograft as a model for human disease and confirming that the compound modulates a phenotype associated with the disease. In some embodiments a biological pathway or process which is modulated by the compound is identified in patient-derived cells cultured in a presently disclosed three-dimensional hydrogel. In some embodiments a biological pathway or process which is modulated by the compound is identified in a presently disclosed patient-derived xenograft. In some embodiments a molecular target of a compound is identified in patient-derived cells cultured in the presently disclosed three-dimensional hydrogels, e.g., using genetic approaches, chemical genetic approaches, biochemical approaches, or a combination thereof. In some embodiments one or more analogs of the compound are synthesized. In some embodiments the compound or an analog of the compound serves as a candidate therapeutic agent for treating a specific patient.

In some aspects, methods patient-derived xenografts may comprise neural cells and/or tissues derived from a patient suffering from a neurodegenerative disease or other disorder affecting the nervous system. In some embodiments they may be used in identifying druggable targets and/or testing candidate therapeutic agents for treating such disorders, as described herein.

### X. Certain Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

The following abbreviations contained herein are defined as follows: 3D means three-dimensional; AFM means atomic force microscopy; BPE means bovine pituitary extract; ECM means extracellular matrix; H&E means hematoxylin and eosin; IF means immunofluorescence; IHC means immunohistochemistry; MaSC means mammary stem cell; and, TDLU means terminal ductal-lobular unit.

“Agent” is used herein to refer to any substance, compound (e.g., molecule), supramolecular complex, material, or combination or mixture thereof. A compound may be any agent that can be represented by a chemical formula, chemical structure, or sequence. Examples of agents, include, e.g., small molecules, polypeptides, nucleic acids (e.g., RNAi agents, antisense oligonucleotide, aptamers), lipids, polysaccharides, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

An “analog” of a first agent refers to a second agent that is structurally and/or functionally similar to the first agent. A “structural analog” of a first agent is an analog that is structurally similar to the first agent. A structural analog of an agent may have substantially similar physical, chemical, biological, and/or pharmacological propert(ies) as the agent or may differ in at least one physical, chemical, biological, or pharmacological property. In some embodiments at least one such property may be altered in a manner that renders the analog more suitable for a purpose of interest. In some embodiments a structural analog of an agent differs from the agent in that at least one atom, functional group, or substructure of the agent is replaced by a different atom, functional group, or substructure in the analog. In some embodiments, a structural analog of an agent differs from the agent in that at least one hydrogen or substituent present in the agent is replaced by a different moiety (e.g., a different substituent) in the analog. In some embodiments an analog may comprise a moiety that reacts with a target to form a covalent bond.

The terms “assessing”, “determining”, “evaluating”, “assaying” are used interchangeably herein to refer to any form of detection or measurement, and include determining whether a substance, signal, disease, condition, etc., is present or not. The result of an assessment may be expressed in qualitative and/or quantitative terms. Assessing may be relative or absolute. “Assessing the presence of” includes determining the amount of something that is present or determining whether it is present or absent.

A “biological process”, may be any set of operations or molecular events, with a defined beginning and end, pertinent to the functioning of integrated living units, e.g., cells, tissues, organs, and organisms. Typically it is a series of events accomplished by one or more ordered assemblies of molecular functions. A “biological pathway” may be any series of actions and/or interactions by and among molecules in a cell that leads to a certain product or a change in a cell. Typically a biological process encompasses or is carried out via one or more biological pathways. Biological pathways include, for example, pathways pertaining to metabolism, genetic information processing (e.g., transcription, translation, RNA transport, RNA degradation; protein folding, sorting, degradation, post-translational modification; DNA replication and repair), environmental information processing (e.g., membrane transport, signal transduction), and cellular processes (e.g., cell cycle, endocytosis, vesicle trafficking), etc. It will be appreciated that the various afore-mentioned biological processes encompass multiple specific pathways). In some embodiments a biological pathway or process is conserved in that the pathway or process is recognizably present in both yeast and mammalian cells).

“Cellular marker” refers to a molecule (e.g., a protein, RNA, DNA, lipid, carbohydrate), complex, or portion thereof, the presence, absence, or level of which in or on a cell (e.g., at least partly exposed at the cell surface) characterizes, indicates, or identifies one or more cell type(s), cell lineage(s), or tissue type(s) or characterizes, indicates, or identifies a particular state (e.g., a diseased or physiological state such as apoptotic or non-apoptotic, a differentiation state, a stem cell state). In some embodiments a cellular marker comprises the presence, absence, or level of a particular modification of a molecule or complex, e.g., a co- or post-translational modification of a protein. A level may be reported in a variety of different ways, e.g., high/low; +/−; numerically, etc. The presence, absence, or level of certain cellular marker(s) may indicate a particular physiological or diseased state of a patient, organ, tissue, or cell. It will be understood that multiple cellular markers may be assessed to, e.g., identify or isolate a cell type of interest, diagnose a disease, etc. In some embodiments between 2 and 10 cellular markers may be assessed. A cellular marker present on or at the surface of cells may be referred to as a “cell surface marker” (CSM). It will be understood that a CSM may be only partially exposed at the cell surface. In some embodiments a CSM or portion thereof is accessible to a specific binding agent present in the environment in which such cell is located, so that the binding agent may be used to, e.g., identify, label, isolate, or target the cell. In some embodiments a CSM is a protein at least part of which is located outside the plasma membrane of a cell. Examples of CSMs include receptors with an extracellular domain, channels, and cell adhesion molecules. In some embodiments, a receptor is a growth factor receptor, hormone receptor (e.g., estrogen receptor, progesterone receptor, a glucocorticoid receptor, and/or an androgen receptor), integrin receptor, folate receptor, or transferrin receptor. A cellular marker may be cell type specific. A cell type specific marker is generally expressed or present at a higher level in or on (at the surface of) a particular cell type or cell types than in or on many or most other cell types (e.g., other cell types in the body or in an artificial environment). In some cases a cell type specific marker is present at detectable levels only in or on a particular cell type of interest and not on other cell types. However, useful cell type specific markers may not be and often are not absolutely specific for the cell type of interest. A cellular marker, e.g., a cell type specific marker, may be present at levels at least 1.5-fold, at least 2-fold or at least 3-fold greater in or on the surface of a particular cell type than in a reference population of cells which may consist, for example, of a mixture containing cells from multiple (e.g., 5-10; 10-20, or more) of different tissues or organs in approximately equal amounts. In some embodiments a cellular marker, e.g., a cell type specific marker, may be present at levels at least 4-5 fold, between 5-10 fold, between 10-fold and 20-fold, between 20-fold and 50-fold, between 50-fold and 100-fold, or more than 100-fold greater than its average expression in a reference population. It will be understood that a cellular marker, e.g., a CSM, may be present in a cell fraction, organelle, cell fragment, or other material originating from a cell in which it is present and may be used to identify, detect, or isolate such material. In general, the level of a cellular marker may be determined using standard techniques such as Northern blotting, in situ hybridization, RT-PCR, sequencing, immunological methods such as immunoblotting, immunohistochemistry, fluorescence detection following staining with fluorescently labeled antibodies (e.g., flow cytometry, fluorescence microscopy), similar methods using non-antibody ligands that specifically bind to the marker, oligonucleotide or cDNA microarray, protein microarray analysis, mass spectrometry, etc. A CSM, e.g., a cell type specific CSM, may be used to detect or isolate cells or as a target in order to deliver an agent to cells. For example, the agent may be linked to a moiety that binds to a CSM. Suitable binding moieties include, e.g., antibodies or ligands, e.g., small molecules, aptamers, or polypeptides. Methods known in the art can be used to separate cells that express a cellular marker, e.g., a CSM, from cells that do not, if desired. In some embodiments a specific binding agent can be used to physically separate cells that express a CSM from cells that do not. In some embodiments, flow cytometry is used to quantify cells that express a cellular marker, e.g., a CSM, or to separate cells that express a cellular marker, e.g., a CSM, from cells that do not. For example, in some embodiments cells are contacted with a fluorescently labeled antibody that binds to the CSM. Fluorescence activated cell sorting (FACS) is then used to separate cells based on fluorescence.

A nucleotide or amino acid residue in a first nucleic acid or protein “corresponds to” a residue in a second nucleic acid or protein if the two residues perform one or more corresponding functions and/or are located at corresponding positions in the first and second nucleic acids or proteins. Corresponding functions are typically the same, equivalent, or substantially equivalent functions, taking into account differences in the environments of the two nucleic acids or proteins as appropriate. Residues at corresponding positions typically align with each other when the sequences of the two nucleic acids or proteins are aligned to maximize identity (allowing the introduction of gaps) using a sequence alignment algorithm or computer program such as those referred to below (see “Identity”) and/or are located at positions such that when the 3-dimensional structures of the proteins is superimposed the residues overlap or occupy structurally equivalent positions and/or form the same, equivalent, or substantially equivalent intramolecular and/or intermolecular contacts or bonds (e.g., hydrogen bonds). The structures may be experimentally determined, e.g., by X-ray crystallography or NMR or predicted, e.g., using structure prediction or molecular modeling software. An alignment may be over the entire length of one or more of the aligned nucleic acid or polypeptide sequences or over at least one protein domain (or nucleotide sequence encoding a protein domain). A “domain” of a protein is a distinct functional and/or structural unit of a protein, e.g., an independently folding unit of a polypeptide chain. In some embodiments a domain is a portion of a protein sequence identified as a domain in the Conserved Domain Database of the NCBI (Marchler-Bauer A et al. (2013), “CDD: conserved domains and protein three-dimensional structure”, Nucleic Acids Res. 41(D1):D384-52). In some embodiments corresponding amino acids are the same in two sequences (e.g., a lysine residue, a threonine residue) or would be considered conservative substitutions for each other. Examples of corresponding residues include (i) the catalytic residues of two homologous enzymes and (ii) sites for post-translational modification of a particular type (e.g., phosphorylation) within corresponding structural or functional domains that have similar effects on the structure or function of homologous proteins.

Computer-aided” as used herein encompasses methods in which a computer system is used to gather, process, manipulate, display, visualize, receive, transmit, store, or otherwise handle information (e.g., data, results, structures, sequences, etc.). A method may comprise causing the processor of a computer to execute instructions to gather, process, manipulate, display, receive, transmit, or store data or other information. The instructions may be embodied in a computer program product comprising a computer-readable medium.

“Detection reagent” refers to an agent that is useful to specifically detect a gene product or other analyte of interest, e.g., an agent that specifically binds to the gene product or other analyte. Examples of agents useful as detection reagents include, e.g., nucleic acid probes or primers that hybridize to RNA or DNA to be detected, antibodies, aptamers, or small molecule ligands that bind to polypeptides to be detected, and the like. In some embodiments a detection reagent comprises a label. In some embodiments a detection reagent is attached to a support. Such attachment may be covalent or noncovalent in various embodiments. Methods suitable for attaching detection reagents or analytes to supports will be apparent to those of ordinary skill in the art. A support may be a substantially planar or flat support or may be a particulate support, e.g., an approximately spherical support such as a microparticle (also referred to as a “bead”, “microsphere”), nanoparticle (or like terms), or population of microparticles. In some embodiments a support is a slide, chip, or filter. In some embodiments a support is at least a portion of an inner surface of a well or other vessel, channel, flow cell, or the like. A support may be rigid, flexible, solid, or semi-solid (e.g., gel). A support may be comprised of a variety of materials such as, for example, glass, quartz, plastic, metal, silicon, agarose, nylon, or paper. A support may be at least in part coated, e.g., with a polymer or substance comprising a reactive functional group suitable for attaching a detection reagent or analyte thereto.

“Druggable target” refers to a biological molecule, e.g., a protein or RNA, the level or activity of which is modulatable (capable of being modulated) by a small molecule. In certain embodiments a druggable target is a biological molecule for which at least one small molecule modulator has been identified. In certain embodiments such modulation is detectable in a cell-free assay, e.g., a protein activity assay. I n certain embodiments such modulation is detectable in a cell-based assay using a cell that expresses the target. Any suitable assay may be used. One of ordinary skill in the art will be aware of many suitable assays for measuring protein activity and will be able to select an appropriate assay taking into account the known or predicted activit(ies) of the protein. The activity may, for example, be a binding activity, catalytic activity, transporter activity, or any other biological activity. In some embodiments modulation of a target may be detected by at least partial reversal of a phenotype induced by overexpression of the target or by deletion of the gene that encodes the target. In certain embodiments a druggable target is a biological molecule such as a protein or RNA that is known to or is predicted to bind with high affinity to at least one small molecule. In certain embodiments a protein is predicted to be “druggable” if it is a member of a protein family for which other members of the family are known to be modulated by or bind to one or more small molecules. In certain embodiments a protein is predicted to be “druggable” if it has an enzymatic activity that is amenable to the identification of modulators using a cell-free assay. In some embodiments the protein can be produced or purified in active form and has at least one known substrate that can be used to measure its activity.

An “effective amount” or “effective dose” of an agent (or composition containing such agent) refers to the amount sufficient to achieve a desired biological and/or pharmacological effect, e.g., when delivered to a cell or organism according to a selected administration form, route, and/or schedule. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered to a subject in a single dose, or through use of multiple doses, in various embodiments.

The term “expression” encompasses the processes by which nucleic acids (e.g., DNA) are transcribed to produce RNA, and (where applicable) RNA transcripts are processed and translated into polypeptides.

The term “gene product” (also referred to herein as “gene expression product” or “expression product”) encompasses products resulting from expression of a gene, such as RNA transcribed from a gene and polypeptides arising from translation of such RNA. It will be appreciated that certain gene products may undergo processing or modification, e.g., in a cell. For example, RNA transcripts may be spliced, polyadenylated, etc., prior to mRNA translation, and/or polypeptides may undergo co-translational or post-translational processing such as removal of secretion signal sequences, removal of organelle targeting sequences, or modifications such as phosphorylation, fatty acylation, etc. The term “gene product” encompasses such processed or modified forms. Genomic, mRNA, polypeptide sequences from a variety of species, including human, are known in the art and are available in publicly accessible databases such as those available at the National Center for Biotechnology Information (www.ncbi.nih.gov) or Universal Protein Resource (www.uniprot.org). Databases include, e.g., GenBank, RefSeq, Gene, UniProtKB/SwissProt, UniProtKB/Trembl, and the like. In general, sequences, e.g., mRNA and polypeptide sequences, in the NCBI Reference Sequence database may be used as gene product sequences for a gene of interest. It will be appreciated that multiple alleles of a gene may exist among individuals of the same species. For example, differences in one or more nucleotides (e.g., up to about 1%, 2%, 3-5% of the nucleotides) of the nucleic acids encoding a particular protein may exist among individuals of a given species. Due to the degeneracy of the genetic code, such variations often do not alter the encoded amino acid sequence, although DNA polymorphisms that lead to changes in the sequence of the encoded proteins can exist. Examples of polymorphic variants can be found in, e.g., the Single Nucleotide Polymorphism Database (dbSNP), available at the NCBI website at www.ncbi.nlm.nih.gov/projects/SNP/. (Sherry S T, et al. (2001). “dbSNP: the NCBI database of genetic variation”. Nucleic Acids Res. 29 (1): 308-311; Kitts A, and Sherry S, (2009). The single nucleotide polymorphism database (dbSNP) of nucleotide sequence variation in The NCBI Handbook [Internet]. McEntyre J, Ostell J, editors. Bethesda (Md.): National Center for Biotechnology Information (US); 2002 (www.ncbi.nlm.nih.gov/bookshelf/br.fcgi?book=handbook&part=ch5). Multiple isoforms of certain proteins may exist, e.g., as a result of alternative RNA splicing or editing. In general, where aspects of this disclosure pertain to a gene or gene product, embodiments pertaining to allelic variants or isoforms are encompassed, if applicable, unless indicated otherwise. Certain embodiments may be directed to particular sequence(s), e.g., particular allele(s) or isoform(s).

“Identity” or “percent identity” is a measure of the extent to which the sequence of two or more nucleic acids or polypeptides is the same. The percent identity between a sequence of interest A and a second sequence B may be computed by aligning the sequences, allowing the introduction of gaps to maximize identity, determining the number of residues (nucleotides or amino acids) that are opposite an identical residue, dividing by the minimum of TGA and TGB (here TGA and TGB are the sum of the number of residues and internal gap positions in sequences A and B in the alignment), and multiplying by 100. When computing the number of identical residues needed to achieve a particular percent identity, fractions are to be rounded to the nearest whole number. Sequences can be aligned with the use of a variety of computer programs known in the art. For example, computer programs such as BLAST2, BLASTN, BLASTP, Gapped BLAST, etc., may be used to generate alignments and/or to obtain a percent identity. The algorithm of Karlin and Altschul (Karlin and Altschul, Proc. Natl. Acad. Sci. USA 87:22264-2268, 1990) modified as in Karlin and Altschul, Proc. Natl. Acad Sci. USA 90:5873-5877, 1993 is incorporated into the NBLAST and XBLAST programs of Altschul et al. (Altschul, et al., J. Mol. Biol. 215:403-410, 1990). In some embodiments, to obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. See the Web site having URL www.ncbi.nlm.nih.gov and/or McGinnis, S. and Madden, T L, W20-W25 Nucleic Acids Research, 2004, Vol. 32, Web server issue. Other suitable programs include CLUSTALW (Thompson J D, Higgins D G, Gibson T J, Nuc Ac Res, 22:4673-4680, 1994) and GAP (GCG Version 9.1; which implements the Needleman & Wunsch, 1970 algorithm (Needleman S B, Wunsch C D, J Mol Biol, 48:443-453, 1970.) Percent identity may be evaluated over a window of evaluation. In some embodiments a window of evaluation may have a length of at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, e.g., 100%, of the length of the shortest of the sequences being compared. In some embodiments a window of evaluation is at least 100; 200; 300; 400; 500; 600; 700; 800; 900; 1,000; 1,200; 1,500; 2,000; 2,500; 3,000; 3,500; 4,000; 4,500; or 5,000 amino acids. In some embodiments no more than 20%, 10%, 5%, or 1% of positions in either sequence or in both sequences over a window of evaluation are occupied by a gap. In some embodiments no more than 20%, 10%, 5%, or 1% of positions in either sequence or in both sequences are occupied by a gap.

“Isolated” means 1) separated from at least some of the components with which it is usually associated in nature; 2) prepared or purified by a process that involves the hand of man; and/or 3) not occurring in nature, e.g., present in an artificial environment. In some embodiments an isolated cell is a cell that has been removed from a subject, generated in vitro, separated from at least some other cells in a cell cluster or sample, or that remains after at least some other cells in a cell cluster or sample have been removed or eliminated.

The term “label” (also referred to as “detectable label”) refers to any moiety that facilitates detection and, optionally, quantification, of an entity that comprises it or to which it is attached. In general, a label may be detectable by, e.g., spectroscopic, photochemical, biochemical, immunochemical, electrical, optical, chemical or other means. In some embodiments a detectable label produces an optically detectable signal (e.g., emission and/or absorption of light), which can be detected e.g., visually or using suitable instrumentation such as a light microscope, a spectrophotometer, a fluorescence microscope, a fluorescent sample reader, a fluorescence activated cell sorter, a camera, or any device containing a photodetector. Labels that may be used in various embodiments include, e.g., organic materials (including organic small molecule fluorophores (sometimes termed “dyes”), quenchers (e.g., dark quenchers), polymers, fluorescent proteins); enzymes; inorganic materials such as metal chelates, metal particles, colloidal metal, metal and semiconductor nanocrystals (e.g., quantum dots); compounds that exhibit luminescensce upon enzyme-catalyzed oxidation such as naturally occurring or synthetic luciferins (e.g., firefly luciferin or coelenterazine and structurally related compounds); haptens (e.g., biotin, dinitrophenyl, digoxigenin); radioactive atoms (e.g., radioisotopes such as 3H, 14 C, 32P, 33P, 35S, 1251), stable isotopes (e.g., 13 C, 2H); magnetic or paramagnetic molecules or particles, etc. Fluorescent dyes include, e.g., acridine dyes; BODIPY, coumarins, cyanine dyes, napthalenes (e.g., dansyl chloride, dansyl amide), xanthene dyes (e.g., fluorescein, rhodamines), and derivatives of any of the foregoing. Examples of fluorescent dyes include Cy3, Cy3.5, Cy5, Cy5.5, Cy7, Alexa® Fluor dyes, DyLight® Fluor dyes, FITC, TAMRA, Oregon Green dyes, Texas Red, to name but a few. Fluorescent proteins include green fluorescent protein (GFP), blue, sapphire, yellow, red, orange, and cyan fluorescent proteins and fluorescent variants such as enhanced GFP (eGFP), mFruits such as mCherry, mTomato, mStrawberry; R-Phycoerythrin, etc. Enzymes useful as labels include, e.g., enzymes that act on a substrate to produce a colored, fluorescent, or luminescent substance. Examples include luciferases, beta-galactosidase, horseradish peroxidase, and alkaline phosphatase. Luciferases include those from various insects (e.g., fireflies, beetles) and marine organisms (e.g., cnidaria such as Renilla (e.g., Renilla reniformis, copepods such as Gaussia (e.g., Gaussia princeps) or Metridia (e.g., Metridia longa, Metridia pacifica), and modified versions of the naturally occurring proteins. A wide variety of systems for labeling and/or detecting labels or labeled entities are known in the art. Numerous detectable labels and methods for their use, detection, modification, and/or incorporation into or conjugation (e.g., covalent or noncovalent attachment) to biomolecules such as nucleic acids or proteins, etc., are described in Iain Johnson, I., and Spence, M. T. Z. (Eds.), The Molecular Probes® Handbook—A Guide to Fluorescent Probes and Labeling Technologies. 11th edition (Life Technologies/Invitrogen Corp.) available online on the Life Technologies website at http://www.invitrogen.com/site/us/en/home/References/Molecular-Probes-The-Handbook.html and Hermanson, G T., Bioconjugate Techniques, 2nd ed., Academic Press (2008). Many labels are available as derivatives that are attached to or incorporate a reactive functional group so that the label can be conveniently conjugated to a biomolecule or other entity of interest that comprises an appropriate second functional group (which second functional group may either occur naturally in the biomolecule or may be introduced during or after synthesis). For example, an active ester (e.g., a succinimidyl ester), carboxylate, isothiocyanate, or hydrazine group can be reacted with an amino group; a carbodiimide can be reacted with a carboxyl group; a maleimide, iodoacetamide, or alkyl bromide (e.g., methyl bromide) can be reacted with a thiol (sulfhydryl); an alkyne can be reacted with an azide (via a click chemistry reaction such as a copper-catalyzed or copper-free azide-alkyne cycloaddition). Thus, for example, an N-hydroxysuccinide (NHS)-functionalized derivative of a fluorophore or hapten (such as biotin) can be reacted with a primary amine such as that present in a lysine side chain in a protein or in an aminoallyl-modified nucleotide incorporated into a nucleic acid during synthesis. A label may be directly attached to an entity or may be attached to an entity via a spacer or linking group, e.g., an alkyl, alkylene, aminoallyl, aminoalkynyl, or oligoethylene glycol spacer or linking group, which may have a length of, e.g., between 1 and 4, 4-8, 8-12, 12-20 atoms, or more in various embodiments. A label or labeled entity may be directly detectable or indirectly detectable in various embodiments. A label or labeling moiety may be directly detectable (i.e., it does not require any further reaction or reagent to be detectable, e.g., a fluorophore is directly detectable) or it may be indirectly detectable (e.g., it is rendered detectable through reaction or binding with another entity that is detectable, e.g., a hapten is detectable by immunostaining after reaction with an appropriate antibody comprising a reporter such as a fluorophore or enzyme; an enzyme acts on a substrate to generate a directly detectable signal). A label may be used for a variety of purposes in addition to or instead of detecting a label or labeled entity. For example, a label can be used to isolate or purify a substance comprising the label or having the label attached thereto. The term “labeled” is used herein to indicate that an entity (e.g., a molecule, probe, cell, tissue, etc.) comprises or is physically associated with (e.g., via a covalent bond or noncovalent association) a label, such that the entity can be detected. In some embodiments a detectable label is selected such that it generates a signal that can be measured and whose intensity is related to (e.g., proportional to) the amount of the label. In some embodiments two or more different labels or labeled entities are used or present in a composition. In some embodiments the labels may be selected to be distinguishable from each other. For example, they may absorb or emit light of different wavelengths. In some embodiments the labels may be selected to interact with each other. For example, a first label may be a donor molecule that transfers energy to a second label, which serves as an acceptor molecule through nonradiative dipole—dipole coupling as in resonance energy transfer (RET), e.g., Förster resonance energy transfer (FRET, also commonly called fluorescence resonance energy transfer).

“Gain of function” generally refers to acquisition of a new, altered, and/or abnormal function or increased function as compared with a reference. The reference may be, e.g., a level or average level of function possessed by a normal gene product (e.g., a gene product whose sequence is the same as a reference sequence) or found in healthy cell(s) or subject(s). An average may be taken across any number of values. In certain embodiments the reference level may be the upper limit of a reference range. In certain embodiments the function may be increased by at least 10%, 20%, 30%, 40%, 50%, 60%, 70%, 80%, 90%, 95%, 99%, or 100% of the reference level. In certain embodiments the function may be increased by between 1 to 2-fold, 2 to 5-fold, 5 to 10-fold, 10 to 20-fold, 20 to 50-fold, 50 to 100-fold, or more, of the reference level. In certain embodiments the function may be increased to a level or within a range that has a statistically significant correlation with or demonstrated causative relationship with a disease (e.g., cancer, e.g., cancers of epithelial origin, e.g., mammary cancer). A “gain of function” mutation in a gene results in a change in a gene product of the gene or increases the expression level of the gene product, such that it gains a new and abnormal function or an abnormally increased function as compared with a gene product of a normal gene. The function may be new in that it is distinct from the activit(ies) of the normal gene product or may result from an increase in or dysregulation of a normal activity of the gene product. The altered gene product encoded by a gene harboring a gain of function mutation may, for example, have one or more altered residues that causes the gene product to have the ability to interact with different cellular molecules or structures than does the normal gene product or causes the gene product to be mislocalized or dysregulated. For purposes hereof, gain of function mutations encompass dominant negative mutations. Dominant negative mutations result in an altered gene product that lacks a function of the normal gene product and acts antagonistically to the normal gene product by, for example, competing with the normal gene product in a context such as a binding partner, ligand, component of a multimolecular complex (e.g., an oligomer), or substrate but failing to fulfill the normal function of the gene product in that context. The altered gene product encoded by a gene harboring a dominant negative mutation may, for example, be a truncated or otherwise altered form of the normal gene product that retains sufficient structure to compete with the normal gene product. In some embodiments a phenotype or disease resulting from a gain of function mutation in a diploid cell or organism has an autosomal dominant inheritance pattern. A “function” may be any biological activity of a gene product. A biological activity may be, for example, catalyzing a particular reaction, binding to or transporting a particular molecule or complex, participating in or interfering with a biological process carried out by a cell or cells or within a subject, etc. The particular function(s) resulting from a gain of function mutation or lost due to a loss of function mutation may or may not be known.

“Loss of function” generally refers to reduction of function or absence of function as compared with a reference level. The reference level may be, e.g., a normal or average level of function possessed by a normal gene product or found in a healthy cell or subject. In certain embodiments the reference level may be the lower limit of a reference range. In certain embodiments the function may be reduced by at least 10%, 20%, 30%, 40%, 50%, 60%, 70%, 80%, 90%, 95%, 99%, or 100% of the reference level. A “loss of function” mutation in a gene refers to a mutation that causes loss (reduction or absence) of at least one function normally provided by a gene product of the gene. A loss of function mutation in a gene may result in a reduced total level of a gene product of the gene in a cell or subject that has the mutation (e.g., due to reduced expression of the gene, reduced stability of the gene product, or both), reduced activity per molecule of the gene product encoded by the mutant gene, or both. The reduction in expression, level, activity per molecule, or total function may be partial or complete. A mutation that confers a complete loss of function, or an allele harboring such a mutation, may be referred to as a null mutation or null allele, respectively. In some embodiments a loss of function mutation in a gene results in a reduction of at least 10%, 20%, 30%, 40%, 50%, 60%, 70%, 80%, 90%, 95%, 99%, or 100% in the level or activity of a gene product of the mutant gene, as compared with level or activity of a gene product encoded by a normal allele of the gene. A loss of function mutation may be an insertion, deletion, or point mutation. For example, a point mutation may introduce a premature stop codon, resulting in a truncated version of the normal gene product that lacks at least a portion of a domain that contributes to or is essential for activity, such as a catalytic domain or binding domain, or may alter an amino acid that contributes to or is essential for activity, such as a catalytic residue, site of post-translational modification, etc. In some embodiments a phenotype or disease resulting from a loss of function mutation in a diploid cell or organism has an autosomal recessive inheritance pattern.

“Modulate” as used herein means to decrease (e.g., inhibit, reduce, suppress) or increase (e.g., stimulate, activate, enhance) a level, response, property, activity, pathway, or process. A “modulator” is an agent capable of modulating a level, response, property, activity, pathway, or process. A modulator may be an inhibitor, antagonist, activator, or agonist. In some embodiments modulation may refer to an alteration, e.g., inhibition or increase, of the relevant level, response, property, activity, pathway, or process by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%.

“Nucleic acid” is used interchangeably with “polynucleotide” and encompasses polymers of nucleotides. “Oligonucleotide” refers to a relatively short nucleic acid, e.g., typically between about 4 and about 100 nucleotides (nt) long, e.g., between 8-60 nt or between 10-40 nt long. Nucleotides include, e.g., ribonucleotides or deoxyribonucleotides. In some embodiments a nucleic acid comprises or consists of DNA or RNA. In some embodiments a nucleic acid comprises or includes only standard nucleobases (often referred to as “bases”). The standard bases are cytosine, guanine, adenine (which are found in DNA and RNA), thymine (which is found in DNA) and uracil (which is found in RNA), abbreviated as C, G, A, T, and U, respectively. In some embodiments a nucleic acid may comprise one or more non-standard nucleobases, which may be naturally occurring or non-naturally occurring (i.e., artificial; not found in nature) in various embodiments. In some embodiments a nucleic acid may comprise one or more chemically or biologically modified bases (e.g., alkylated (e.g., methylated) bases), modified sugars (e.g., 2′-O-alkyribose (e.g., 2′-O methylribose), 2′-fluororibose, arabinose, or hexose), modified phosphate groups or modified internucleoside linkages (i.e., a linkage other than a phosphodiester linkage between consecutive nucleosides, e.g., between the 3′ carbon atom of one sugar molecule and the 5′ carbon atom of another), such as phosphorothioates, 5′-N-phosphoramidites, alkylphosphonates, phosphorodithioates, phosphate esters, alkylphosphonothioates, phosphoramidates, carbamates, carbonates, phosphate triesters, acetamidates, carboxymethyl esters and peptide bonds). In some embodiments a modified base has a label (e.g., a small organic molecule such as a fluorophore dye) covalently attached thereto. In some embodiments the label or a functional group to which a label can be attached is incorporated or attached at a position that is not involved in Watson-Crick base pairing such that a modification at that position will not significantly interfere with hybridization. For example the C-5 position of UTP and dUTP is not involved in Watson-Crick base-pairing and is a useful site for modification or attachment of a label. In some embodiments a “modified nucleic acid” is a nucleic acid characterized in that (1) at least two of its nucleosides are covalently linked via a non-standard internucleoside linkage (i.e., a linkage other than a phosphodiester linkage between the 5′ end of one nucleotide and the 3′ end of another nucleotide); (2) it incorporates one or more modified nucleotides (which may comprise a modified base, sugar, or phosphate); and/or (3) a chemical group not normally associated with nucleic acids in nature has been covalently attached to the nucleic acid. Modified nucleic acids include, e.g., locked nucleic acids (in which one or more nucleotides is modified with an extra bridge connecting the 2′ oxygen and 4′ carbon i.e., at least one 2′-O,4′-C-methylene-β-D-ribofuranosyl nucleotide), morpholinos (nucleic acids in which at least some of the nucleobases are bound to morpholine rings instead of deoxyribose or ribose rings and linked through phosphorodiamidate groups instead of phosphates), and peptide nucleic acids (in which the backbone is composed of repeating N-(2-aminoethyl)-glycine units linked by peptide bonds and the nucleobases are linked to the backbone by methylene carbonyl bonds). Modifications may occur anywhere in a nucleic acid. A modified nucleic acid may be modified throughout part or all of its length, may contain alternating modified and unmodified nucleotides or internucleoside linkages, or may contain one or more segments of unmodified nucleic acid and one or more segments of modified nucleic acid. A modified nucleic acid may contain multiple different modifications, which may be of different types. A modified nucleic acid may have increased stability (e.g., decreased susceptibility to spontaneous or nuclease-catalyzed hydrolysis) or altered hybridization properties (e.g., increased affinity or specificity for a target, e.g., a complementary nucleic acid), relative to an unmodified counterpart having the same nucleobase sequence. In some embodiments a modified nucleic acid comprises a modified nucleobase having a label covalently attached thereto. Non-standard nucleotides and other nucleic acid modifications known in the art as being useful in the context of nucleic acid detection reagents, RNA interference (RNAi), aptamer, or antisense-based molecules for research or therapeutic purposes are contemplated for use in various embodiments of the instant invention. See, e.g., The Molecular Probes® Handbook—A Guide to Fluorescent Probes and Labeling Technologies (cited above), Bioconjugate Techniques (cited above), Crooke, S T (ed.) Antisense drug technology: principles, strategies, and applications, Boca Raton: CRC Press, 2008; Kurrcek. J. (ed.) Therapeutic oligonucleotides, RSC biomolecular sciences. Cambridge: Royal Society of Chemistry, 2008. A nucleic acid can be single-stranded, double-stranded, or partially double-stranded. An at least partially double-stranded nucleic acid can have one or more overhangs, e.g., 5′ and/or 3′ overhang(s). Where a nucleic acid sequence is disclosed herein, it should be understood that its complement and double-stranded form is also disclosed.

A “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. In some embodiments, a non-standard, naturally occurring amino acid is found in mammals. For example, ornithine, citrulline, and homocysteine are naturally occurring non-standard amino acids that have important roles in mammalian metabolism. Examples of non-standard amino acids include, e.g., singly or multiply halogenated (e.g., fluorinated) amino acids, D-amino acids, homo-amino acids, N-alkyl amino acids (other than proline), dehydroamino acids, aromatic amino acids (other than histidine, phenylalanine, tyrosine and tryptophan), and α,α disubstituted amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, etc. Modifications may occur anywhere in a polypeptide, e.g., the peptide backbone, the amino acid side-chains and the amino or carboxyl termini. A given polypeptide may contain many types of modifications. Polypeptides may be branched or they may be cyclic, with or without branching. Polypeptides may be conjugated with, encapsulated by, or embedded within a polymer or polymeric matrix, dendrimer, nanoparticle, microparticle, liposome, or the like. Modification may occur prior to or after an amino acid is incorporated into a polypeptide in various embodiments. Polypeptides may, for example, be purified from natural sources, produced in vitro or in vivo in suitable expression systems using recombinant DNA technology (e.g., by recombinant host cells or in transgenic animals or plants), synthesized through chemical means such as conventional solid phase peptide synthesis, and/or methods involving chemical ligation of synthesized peptides (see, e.g., Kent, S., J Pept Sci., 9(9):574-93, 2003 or U.S. Pub. No. 20040115774), or any combination of the foregoing.

As used herein, the term “toxic agent” refers to a substance that causes damage to cell function or structure or is metabolized or otherwise converted to such a substance when present in a cell or in the environment of a cell. Toxic agents include, e.g., oxidative stressors, nitrosative stressors, proteasome inhibitors, inhibitors of mitochondrial function, ionophores, inhibitors of vacuolar ATPases, inducers of endoplasmic reticulum (ER) stress, and inhibitors of endoplasmic reticulum associated degradation (ERAD). In some embodiments a toxic agent selectively causes damage to nervous system tissue. Toxic agents include agents that are directly toxic and agents that are metabolized to or give rise to substances that are directly toxic. It will be understood that the term “toxic agent” typically refers to agents that are not ordinarily present in a cell's normal environment at sufficient levels to exert detectable damaging effects. Typically they exert damaging effects when present at a relatively low concentration, e.g., at or below 1 mM, e.g., at or below 500 μM, e.g., at or below 100 μM. It will be understood that a toxic agent typically has a threshold concentration below which it does not exert detectable damaging effects. The particular threshold concentration will vary depending on the agent and, potentially, other factors such as cell type, other agents present in the environment, etc. Exemplary threshold concentrations may be in the range of 1 nM to 100 nM, 100 nM to 1 μm, 1 μm to 10 μm, or 10 μm to 100 μm. “Oxidative stressor” refers to an agent that causes an increase in the level of reactive oxygen species and/or a decrease in a biological system's ability to detoxify the reactive species or intermediates generated through their activity or to repair the resulting damage (e.g., damage to DNA or other biomolecules), resulting in impairment to the structure and/or function of the system. “Nitrosative stressor” refers to an agent that causes an increase in the level of reactive nitrogen species and/or a decrease in a biological system's ability to detoxify the reactive species or intermediates generated through their activity or to repair the resulting damage (e.g., damage to DNA or other biomolecules), resulting in impairment to the structure and/or function of the system. Proteasome inhibitors include, e.g., MG-132 (CAS number 133407-82-6) and bortezomib. Inhibitors of mitochondrial function include, e.g., inhibitors of mitochondrial oxidative phosphorylation such as compounds that inhibit any of mitochondrial complexes I-V, e.g., complex I inhibitors. Inhibitors of vacuolar ATPases include, e.g., bafilomycins and concanamycins. Inhibitors of ERAD include, e.g., eeyarestatin I or eeyarestatin II (Fiebiger, E., et al. (2004) Dissection of the dislocation pathway for type I membrane proteins with a new small molecule inhibitor, eeyarestatin. Mol. Biol. Cell 15, 1635-1646).

As used herein, the term “purified” refers to agents that have been separated from most of the components with which they are associated in nature or when originally generated or with which they were associated prior to purification. In general, such purification involves action of the hand of man. Purified agents may be partially purified, substantially purified, or pure. Such agents may be, for example, at least 50%, 60%, 70%, 75%, 80%, 85%, 90%, 95%, 96%, 97%, 98%, 99%, or more than 99% pure. In some embodiments, a nucleic acid, polypeptide, or small molecule is purified such that it constitutes at least 75%, 80%, 85%, 90%, 95%, 96%, 97%, 98%, 99%, or more, of the total nucleic acid, polypeptide, or small molecule material, respectively, present in a preparation. In some embodiments, an organic substance, e.g., a nucleic acid, polypeptide, or small molecule, is purified such that it constitutes at least 75%, 80%, 85%, 90%, 95%, 96%, 97%, 98%, 99%, or more, of the total organic material present in a preparation. Purity may be based on, e.g., dry weight, size of peaks on a chromatography tracing (GC, HPLC, etc.), molecular abundance, electrophoretic methods, intensity of bands on a gel, spectroscopic data (e.g., NMR), elemental analysis, high throughput sequencing, mass spectrometry, or any art-accepted quantification method. In some embodiments, water, buffer substances, ions, and/or small molecules (e.g., synthetic precursors such as nucleotides or amino acids), can optionally be present in a purified preparation. A purified agent may be prepared by separating it from other substances (e.g., other cellular materials), or by producing it in such a manner to achieve a desired degree of purity. In some embodiments “partially purified” with respect to a molecule produced by a cell means that a molecule produced by a cell is no longer present within the cell, e.g., the cell has been lysed and, optionally, at least some of the cellular material (e.g., cell wall, cell membrane(s), cell organelle(s)) has been removed and/or the molecule has been separated or segregated from at least some molecules of the same type (protein, RNA, DNA, etc.) that were present in the lysate.

A “reference range” for a value, e.g., a reference range for a value associated with a gene product, biological activity, cell, or subject, refers to the range into which 95%, or in some embodiments 90%, of the values measured from normal or control gene products or healthy cells or subjects fall, or a range that encompasses only values that do not have a statistically significant correlation with diseases in general (e.g., cancer, e.g. cancers of epithelial origin, e.g., mammary cancer) or with a particular disease of interest (e.g., cancer, e.g., cancers of epithelial origin, e.g., mammary cancer) as compared to the average value in healthy cells or subjects. A reference range may be established from a representative sample of a population. In some embodiments a reference range may be established by performing measurements on gene products or healthy cells obtained from multiple subjects who are apparently healthy or at least free of a particular disease of interest (e.g., cancer, e.g., cancers of epithelial origin, e.g., mammary cancer) and not known to be at increased risk of developing the disease.

The term “sample” may be used to generally refer to an amount or portion of something. A sample may be a smaller quantity taken from a larger amount or entity; however, a complete specimen may also be referred to as a sample where appropriate. A sample is often intended to be similar to and representative of a larger amount of the entity of which it is a sample. In some embodiments a sample is a quantity of a substance that is or has been or is to be provided for assessment (e.g., testing, analysis, measurement) or use. A sample may be any biological specimen. In some embodiments a sample comprises a body fluid such as blood, cerebrospinal fluid, (C SF), sputum, lymph, mucus, saliva, a glandular secretion, or urine. In some embodiments a sample comprises cells, tissue, or cellular material (e.g., material derived from cells, such as a cell lysate or fraction thereof). A sample may be obtained from (i.e., originates from, was initially removed from) a subject. Methods of obtaining biological samples from subjects are known in the art and include, e.g., tissue biopsy, such as excisional biopsy, incisional biopsy, core biopsy; fine needle aspiration biopsy; surgical excision, brushings; lavage; or collecting body fluids that may contain cells, such as blood, sputum, lymph, mucus, saliva, or urine. A sample is often intended to be similar to and representative of a larger amount of the entity of which it is a sample. A sample of a cell line comprises a limited number of cells of that cell line. In some embodiments a sample may be obtained from an individual who has been diagnosed with or is suspected of having a disease (e.g., cancer, e.g., cancer of epithelial origin, e.g., mammary cancer). In some embodiments a sample is obtained from skin or blood. In some embodiments a sample contains at least some intact cells. In some embodiments a sample retains at least some of the microarchitecture of a tissue from which it was removed. A sample may be subjected to one or more processing steps, e.g., after having been obtained from a subject, and/or may be split into one or more portions. The term sample encompasses processed samples, portions of samples, etc., and such samples are, where applicable, considered to have been obtained from the subject from whom the initial sample was removed. A sample may be procured directly from a subject, or indirectly, e.g., by receiving the sample from one or more persons who procured the sample directly from the subject, e.g., by performing a biopsy, surgery, or other procedure on the subject. In some embodiments a sample may be assigned an identifier (ID), which may be used to identify the sample as it is transported, processed, analyzed, and/or stored. In some embodiments the sample ID corresponds to the subject from whom the sample originated and allows the sample and/or results obtained by assessing the sample to be matched with the subject. In some embodiments the sample has an identifier affixed thereto.

A “small molecule” as used herein, is an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

“Specific binding” generally refers to a physical association between a target molecule (e.g., a polypeptide) or complex and a binding agent such as an antibody, aptamer or ligand. The association is typically dependent upon the presence of a particular structural feature of the target such as an antigenic determinant, epitope, binding pocket or cleft, recognized by the binding agent. For example, if an antibody is specific for epitope A, the presence of a polypeptide containing epitope A or the presence of free unlabeled A in a reaction containing both free labeled A and the binding agent that binds thereto, will typically reduce the amount of labeled A that binds to the binding agent. It is to be understood that specificity need not be absolute but generally refers to the context in which the binding occurs. For example, it is well known in the art that antibodies may in some instances cross-react with other epitopes in addition to those present in the target. Such cross-reactivity may be acceptable depending upon the application for which the antibody is to be used. One of ordinary skill in the art will be able to select binding agents, e.g., antibodies, aptamers, or ligands, having a sufficient degree of specificity to perform appropriately in any given application (e.g., for detection of a target molecule). It is also to be understood that specificity may be evaluated in the context of additional factors such as the affinity of the binding agent for the target versus the affinity of the binding agent for other targets, e.g., competitors. If a binding agent exhibits a high affinity for a target molecule that it is desired to detect and low affinity for non-target molecules, the binding agent will likely be an acceptable reagent. Once the specificity of a binding agent is established in one or more contexts, it may be employed in other contexts, e.g., similar contexts such as similar assays or assay conditions, without necessarily re-evaluating its specificity. In some embodiments specificity of a binding agent can be tested by performing an appropriate assay on a sample expected to lack the target (e.g., a sample from cells in which the gene encoding the target has been disabled or effectively inhibited) and showing that the assay does not result in a signal significantly different to background. In some embodiments, a first entity (e.g., molecule, complex) is said to “specifically bind” to a second entity if it binds to the second entity with substantially greater affinity than to most or all other entities present in the environment where such binding takes place and/or if the two entities bind with an equilibrium dissociation constant, Kd, of 10-4 or less, e.g., 10-5 M or less, e.g., 10-6 M or less, 10-7 M or less, 10-8 M or less, 10-9 M or less, or 10-10 M or less. Kd can be measured using any suitable method known in the art, e.g., surface plasmon resonance-based methods, isothermal titration calorimetry, differential scanning calorimetry, spectroscopy-based methods, etc. “Specific binding agent” refers to an entity that specifically binds to another entity, e.g., a molecule or molecular complex, which may be referred to as a “target”. “Specific binding pair” refers to two entities (e.g., molecules or molecular complexes) that specifically bind to one another. Examples are biotin-avidin, antibody-antigen, complementary nucleic acids, receptor-ligand, etc.

A “subject” may be any vertebrate organism in various embodiments. A subject may be individual to whom an agent is administered, e.g., for experimental, diagnostic, and/or therapeutic purposes or from whom a sample is obtained or on whom a procedure is performed. In some embodiments a subject is a mammal, e.g. a human, non-human primate, or rodent (e.g., mouse, rat, rabbit). In some embodiments a human subject is at least 50, 60, 70, 80, or 90 years old.

“Treat”, “treating” and similar terms as used herein in the context of treating a subject refer to providing medical and/or surgical management of a subject. Treatment may include, but is not limited to, administering an agent or composition (e.g., a pharmaceutical composition) to a subject. Treatment is typically undertaken in an effort to alter the course of a disease (which term is used to indicate any disease, disorder, syndrome or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. A therapeutic agent may be administered to a subject who has a disease or is at increased risk of developing a disease relative to a member of the general population. In some embodiments a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur or to reduce the severity of the disease should it occur. The subject may have been identified as being at risk of developing the disease (e.g., at increased risk relative to the general population or as having a risk factor that increases the likelihood of developing the disease.

A “variant” of a particular polypeptide or polynucleotide has one or more additions, substitutions, and/or deletions with respect to the polypeptide or polynucleotide, which may be referred to as the “original polypeptide” or “original polynucleotide”, respectively. An addition may be an insertion or may be at either terminus. A variant may be shorter or longer than the original polypeptide or polynucleotide. The term “variant” encompasses “fragments”. A “fragment” is a continuous portion of a polypeptide or polynucleotide that is shorter than the original polypeptide. In some embodiments a variant comprises or consists of a fragment. In some embodiments a fragment or variant is at least 20%, 30%, 40%, 50%, 60%, 70%, 80%, 90%, 95%, 96%, 97%, 98%, 99%, or more as long as the original polypeptide or polynucleotide. A fragment may be an N-terminal, C-terminal, or internal fragment. In some embodiments a variant polypeptide comprises or consists of at least one domain of an original polypeptide. In some embodiments a variant polypeptide or polynucleotide comprises or consists of a polypeptide or polynucleotide that is at least 70%, 80%, 90%, 95%, 96%, 97%, 98%, 99%, or more identical in sequence to the original polypeptide or polynucleotide over at least 50%, 60%, 70%, 80%, 90%, 95%, 96%, 97%, 98%, 99%, or 100% of the original polypeptide or polynucleotide. In some embodiments the sequence of a variant polypeptide comprises or consists of a sequence that has N amino acid differences with respect to an original sequence, wherein N is any integer up to 1%, 2%, 5%, or 10% of the number of amino acids in the original polypeptide, where an “amino acid difference” refers to a substitution, insertion, or deletion of an amino acid. In some embodiments a substitution is a conservative substitution. Conservative substitutions may be made, e.g., on the basis of similarity in side chain size, polarity, charge, solubility, hydrophobicity, hydrophilicity and/or the amphipathic nature of the residues involved. In some embodiments, conservative substitutions may be made according to Table A, wherein amino acids in the same block in the second column and in the same line in the third column may be substituted for one another other in a conservative substitution. Certain conservative substitutions are substituting an amino acid in one row of the third column corresponding to a block in the second column with an amino acid from another row of the third column within the same block in the second column.

TABLE A

| | | |
|---|---|---|
| Aliphatic | Non-polar | G A P |
| | | I L V |
| | Polar - uncharged | C S T M |
| | | N Q |
| | Polar - charged | D E |
| | | K R |
| Aromatic | | H F W Y |

In some embodiments, proline (P), cysteine (C), or both are each considered to be in an individual group. Within a particular group, certain substitutions may be of particular interest in certain embodiments, e.g., replacements of leucine by isoleucine (or vice versa), serine by threonine (or vice versa), or alanine by glycine (or vice versa).

In some embodiments a variant is a biologically active variant, i.e., the variant at least in part retains at least one activity of the original polypeptide or polynucleotide. In some embodiments a variant at least in part retains more than one or substantially all known biologically significant activities of the original polypeptide or polynucleotide. An activity may be, e.g., a catalytic activity, binding activity, ability to perform or participate in a biological structure or process, etc. In some embodiments an activity of a variant may be at least 10%, 20%, 30%, 40%, 50%, 60%, 70%, 80%, 90%, 95%, or more, of the activity of the original polypeptide or polynucleotide, up to approximately 100%, approximately 125%, or approximately 150% of the activity of the original polypeptide or polynucleotide, in various embodiments. In some embodiments a variant, e.g., a biologically active variant, comprises or consists of a polypeptide at least 95%, 96%, 97%, 98%, 99%, 99.5% or 100% identical to an original polypeptide over at least 70%, 75%, 80%, 85%, 90%, 95%, 96%, 97%, 98%, or 99% or 100% of the original polypeptide. In some embodiments an alteration, e.g., a substitution or deletion, e.g., in a functional variant, does not alter or delete an amino acid or nucleotide that is known or predicted to be important for an activity, e.g., a known or predicted catalytic residue or residue involved in binding a substrate or cofactor. Variants may be tested in one or more suitable assays to assess activity.

A “vector” may be any of a number of nucleic acid molecules or viruses or portions thereof that are capable of mediating entry of, e.g., transferring, transporting, etc., a nucleic acid of interest between different genetic environments or into a cell. The nucleic acid of interest may be linked to, e.g., inserted into, the vector using, e.g., restriction and ligation. Vectors include, for example, DNA or RNA plasmids, cosmids, naturally occurring or modified viral genomes or portions thereof, nucleic acids that can be packaged into viral capsids, mini-chromosomes, artificial chromosomes, etc. Plasmid vectors typically include an origin of replication (e.g., for replication in prokaryotic cells). A plasmid may include part or all of a viral genome (e.g., a viral promoter, enhancer, processing or packaging signals, and/or sequences sufficient to give rise to a nucleic acid that can be integrated into the host cell genome and/or to give rise to infectious virus). Viruses or portions thereof that can be used to introduce nucleic acids into cells may be referred to as viral vectors. Viral vectors include, e.g., adenoviruses, adeno-associated viruses, retroviruses (e.g., lentiviruses), vaccinia virus and other poxviruses, herpes viruses (e.g., herpes simplex virus), and others. Viral vectors may or may not contain sufficient viral genetic information for production of infectious virus when introduced into host cells, i.e., viral vectors may be replication-competent or replication-defective. In some embodiments, e.g., where sufficient information for production of infectious virus is lacking, it may be supplied by a host cell or by another vector introduced into the cell, e.g., if production of virus is desired. In some embodiments such information is not supplied, e.g., if production of virus is not desired. A nucleic acid to be transferred may be incorporated into a naturally occurring or modified viral genome or a portion thereof or may be present within a viral capsid as a separate nucleic acid molecule. A vector may contain one or more nucleic acids encoding a marker suitable for identifying and/or selecting cells that have taken up the vector. Markers include, for example, various proteins that increase or decrease either resistance or sensitivity to antibiotics or other agents (e.g., a protein that confers resistance to an antibiotic such as puromycin, hygromycin or blasticidin), enzymes whose activities are detectable by assays known in the art (e.g., β-galactosidase or alkaline phosphatase), and proteins or RNAs that detectably affect the phenotype of cells that express them (e.g., fluorescent proteins). Vectors often include one or more appropriately positioned sites for restriction enzymes, which may be used to facilitate insertion into the vector of a nucleic acid, e.g., a nucleic acid to be expressed. An expression vector is a vector into which a desired nucleic acid has been inserted or may be inserted such that it is operably linked to regulatory elements (also termed “regulatory sequences”, “expression control elements”, or “expression control sequences”) and may be expressed as an RNA transcript (e.g., an mRNA that can be translated into protein or a noncoding RNA such as an shRNA or miRNA precursor). Expression vectors include regulatory sequence(s), e.g., expression control sequences, sufficient to direct transcription of an operably linked nucleic acid under at least some conditions; other elements required or helpful for expression may be supplied by, e.g., the host cell or by an in vitro expression system. Such regulatory sequences typically include a promoter and may include enhancer sequences or upstream activator sequences. In some embodiments a vector may include sequences that encode a 5′ untranslated region and/or a 3′ untranslated region, which may comprise a cleavage and/or polyadenylation signal. In general, regulatory elements may be contained in a vector prior to insertion of a nucleic acid whose expression is desired or may be contained in an inserted nucleic acid or may be inserted into a vector following insertion of a nucleic acid whose expression is desired. As used herein, a nucleic acid and regulatory element(s) are said to be “operably linked” when they are covalently linked so as to place the expression or transcription of the nucleic acid under the influence or control of the regulatory element(s). For example, a promoter region would be operably linked to a nucleic acid if the promoter region were capable of effecting transcription of that nucleic acid. One of ordinary skill in the art will be aware that the precise nature of the regulatory sequences useful for gene expression may vary between species or cell types, but may in general include, as appropriate, sequences involved with the initiation of transcription, RNA processing, or initiation of translation. The choice and design of an appropriate vector and regulatory element(s) is within the ability and discretion of one of ordinary skill in the art. For example, one of skill in the art will select an appropriate promoter (or other expression control sequences) for expression in a desired species (e.g., a mammalian species) or cell type. A vector may contain a promoter capable of directing expression in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as EFlalpha, ubiquitin (e.g., ubiquitin B or C), globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, a promoter that ordinarily directs transcription by a eukaryotic RNA polymerase II (a “pol II promoter”) or a functional variant thereof is used. In some embodiments, a promoter that ordinarily directs transcription by a eukaryotic RNA polymerase I promoter, e.g., a promoter for transcription of ribosomal RNA (other than 5S rRNA) or a functional variant thereof is used. In some embodiments, a promoter that ordinarily directs transcription by a eukaryotic RNA polymerase III (a “pol III promoter”), e.g., (a U6, H1, 7SK or tRNA promoter or a functional variant thereof) may be used. One of ordinary skill in the art will select an appropriate promoter for directing transcription of a sequence of interest. Examples of expression vectors that may be used in mammalian cells include, e.g., the pcDNA vector series, pSV2 vector series, pCMV vector series, pRSV vector series, pEF1 vector series, Gateway® vectors, etc. Examples of virus vectors that may be used in mammalian cells include, e.g., adenoviruses, adeno-associated viruses, poxviruses such as vaccinia viruses and attenuated poxviruses, retroviruses (e.g., lentiviruses), Semliki Forest virus, Sindbis virus, etc. In some embodiments, regulatable (e.g., inducible or repressible) expression control element(s), e.g., a regulatable promoter, is/are used so that expression can be regulated, e.g., turned on or increased or turned off or decreased. For example, the tetracycline-regulatable gene expression system (Gossen & Bujard, Proc. Natl. Acad. Sci. 89:5547-5551, 1992) or variants thereof (see, e.g., Allen, N, et al. (2000) Mouse Genetics and Transgenics: 259-263; Urlinger, S, et al. (2000). Proc. Natl. Acad. Sci. U.S.A. 97 (14): 7963-8; Zhou, X., et al (2006). Gene Ther. 13 (19): 1382-1390 for examples) can be employed to provide inducible or repressible expression. Other inducible/repressible systems may be used in various embodiments. For example, expression control elements that can be regulated by small molecules such as artificial or naturally occurring hormone receptor ligands (e.g., steroid receptor ligands such as naturally occurring or synthetic estrogen receptor or glucocorticoid receptor ligands), tetracycline or analogs thereof, metal-regulated systems (e.g., metallothionein promoter) may be used in certain embodiments. In some embodiments, tissue-specific or cell type specific regulatory element(s) may be used, e.g., in order to direct expression in one or more selected tissues or cell types. In some embodiments a vector capable of being stably maintained and inherited as an episome in mammalian cells (e.g., an Epstein-Ban virus-based episomal vector) may be used. In some embodiments a vector may comprise a polynucleotide sequence that encodes a polypeptide, wherein the polynucleotide sequence is positioned in frame with a nucleic acid inserted into the vector so that an N- or C-terminal fusion is created. In some embodiments the polypeptide encoded by the polynucleotide sequence may be a targeting peptide. A targeting peptide may comprise a signal sequence (which directs secretion of a protein) or a sequence that directs the expressed protein to a specific organelle or location in the cell such as the nucleus or mitochondria. In some embodiments the polypeptide comprises a tag. A tag may be useful to facilitate detection and/or purification of a protein that contains it. Examples of tags include polyhistidine-tag (e.g., 6×-His tag), glutathione-S-transferase, maltose binding protein, NUS tag, SNUT tag, Strep tag, epitope tags such as V5, HA, Myc, or FLAG. In some embodiments a protease cleavage site is located in the region between the protein encoded by the inserted nucleic acid and the polypeptide, allowing the polypeptide to be removed by exposure to the protease.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

Growth of Human Breast Tissues from Patient Cells in 3D Hydrogel Scaffolds Introduction The ability to grow human tissues in three-dimensional (3D) cultures has proven immensely useful for regenerative medicine as well as for studies of tissue development. Such ‘organoid’ culture systems have been developed for several types of human tissues, including intestine, stomach, kidney, and brain (Lancaster et al., 2013; McCracken et al., 2014; Sato et al., 2009; Takasato et al., 2014). For mammary tissue, 3D cultures were first introduced nearly four decades ago by Emerman and Pitelka, who described a floating collagen matrix that supported the growth of mammary spheroids from primary mouse epithelial cells (Emerman et al., 1977; Emerman and Pitelka, 1977). Subsequently, Bissell and colleagues developed an improved basement membrane (Matrigel) culture, in which mouse epithelial cells generated ducts and lobules, enabling, for the first time, the in vitro study of mammary morphogenesis (Barcellos-Hoff et al., 1989).

While these and similar 3D cultures have contributed valuable insights into murine mammary gland biology (Chen et al., 2014; Ewald et al., 2008; Lee et al., 1985; Lee et al., 1984; Simian et al., 2001; Sternlicht et al., 2005), the mammary tissue of mice is known to differ in important ways from mammary tissue in humans (Cardiff and Wellings, 1999; Visvader, 2009). In an effort to address this problem, several investigators have successfully grown tissues from human mammary cell lines immortalized by transduction with viral oncogenes (Berdichevsky et al., 1994; Debnath et al., 2003; Gudjonsson et al., 2002). However, growing tissues from primary human mammary cells has proven to be more challenging. Tanos and colleagues showed that they could maintain viable primary human mammary tissue fragments in liquid cultures for up to 6 days (Tanos et al., 2013), but this system did not support the initiation or elongation of ducts (likely due to the absence of extra-cellular matrix). Ductal growth is also limited in collagen or basement membrane (Matrigel) 3D cultures of primary human mammary tissue (Pasic et al., 2011; Yang et al., 1987).

Extracellular matrix (ECM) composition exerts a significant influence on the growth of epithelial tissues. The human breast is a hydrated matrix of protein fibrils interwoven within a network of glycosaminoglycan carbohydrate chains. From a structural perspective, the protein components of the ECM—which include laminins, fibronectin, and collagens—provide resistance to tensile forces, while carbodydrates—composed primarily of hyaluronan chains—chelate water to provide resistance to compressive forces. To more fully reflect this complexity, we engineered a novel scaffold that incorporated both protein (collagen, laminins, fibronectin) and carbohydrate components (hyaluronan) of human breast tissue.

When seeded into these scaffolds, primary human mammary cells from patient reduction mammoplasties are able to self-organize, grow and differentiate into mature breast tissues. We anticipate that these cultures will prove useful in future investigations of human mammary tissue morphogenesis and biology.

### Methods

I. Preparation of Primary Patient-Derived Tissue

Reduction mammoplasty tissue samples were mechanically dissociated and then incubated with 3 mg/mL collagenase (Roche) and 0.7 mg/mL hyaluronidase (Sigma Aldrich) at 37 C overnight. Epithelial clusters were disrupted by trituration, washed, and depleted for fibroblasts. Mouse mammary epithelial tissues were prepared using the same protocol.

II. Preparation of Hydrogels

Hydrogels were composed of 1.7 mg/mL collagen I, 10 μg/mL hyaluronan 150 and 500 kDa (Sigma Aldrich), 40 μg/mL laminin isolated from Engelbreth-Holm-Swarm sarcoma cells (Thermo Fisher), and 20 μg/mL fibronectin (Life Technologies), pH 7.3, to which tissue fragments (e.g., single cells and/or clusters of cells) and growth factors were added (see Supporting Methods for details). Hydrogels were produced in a 4-chamber slide (Corning) as a mold, and incubated at 37 C for polymerization. These gels partially polymerized within five minutes and fully solidified within an hour at which time they were detached from the mold. Structures were passaged from one hydrogel to another by dissolving the pad with collagenase and reseeding the structure as if it were a primary tissue fragment. All experiments were performed with at least four independent replicates (n) using samples from at least three patients (k) unless otherwise specified (N=n, k).

III. Lentiviral Production

Lentivirus production was performed as previously described (Gupta et al., 2005). LeGO lentiviral vectors were kindly provided by Kristoffer Riecken (Weber et al., 2011).

IV. Immunofluorescence/Immunohistochemistry

Immunofluorescent (IF) staining was performed as previously described (Sokol et al., 2015). Immunohistochemistry (IHC) staining was performed at the Koch Institute Histology Core using the ThermoScientific IHC Autostainer 360.

IV. Microscopy

Images were captured using a Zeiss LSM 700 (IF), Zeiss Axiophot (IHC), and Nikon TE2000 with a heated stage and 5% CO2 (Time-lapse).

### Results

I. Design of Hydrogels with Features of Human Breast Tissue

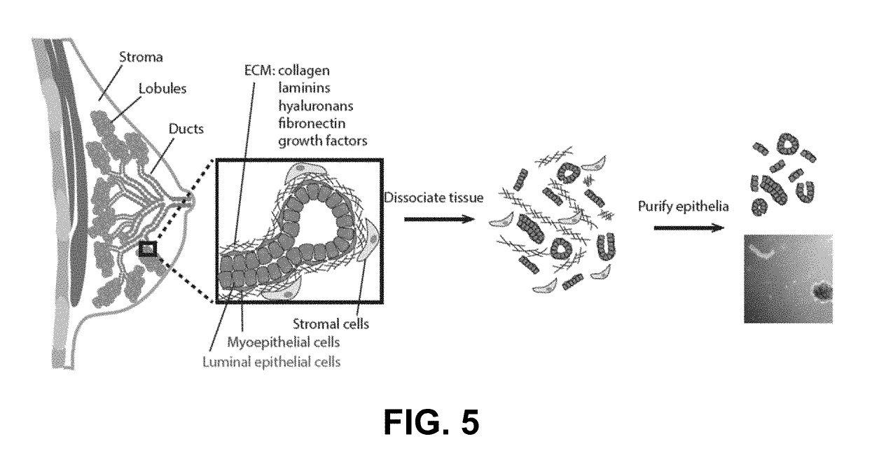

We were interested in engineering a three-dimensional scaffold that could stimulate the growth of human breast tissues, when seeded with cells from patient reduction mammoplasties. We therefore explored hydrogel formulations that contained mammary ECM protein and glycosaminoglycan components found in human breast tissue. We focused our efforts on hybrid hydrogels with defined components, since the presence of serum—which is highly variable and not well defined—would limit their usefulness in regenerative and basic research applications. We evaluated hydrogel formulations by assessing their ability to support the growth of breast tissue fragments containing 50-100 cells, dissociated from patient-derived reduction mammoplasty tissues (FIG. 5).

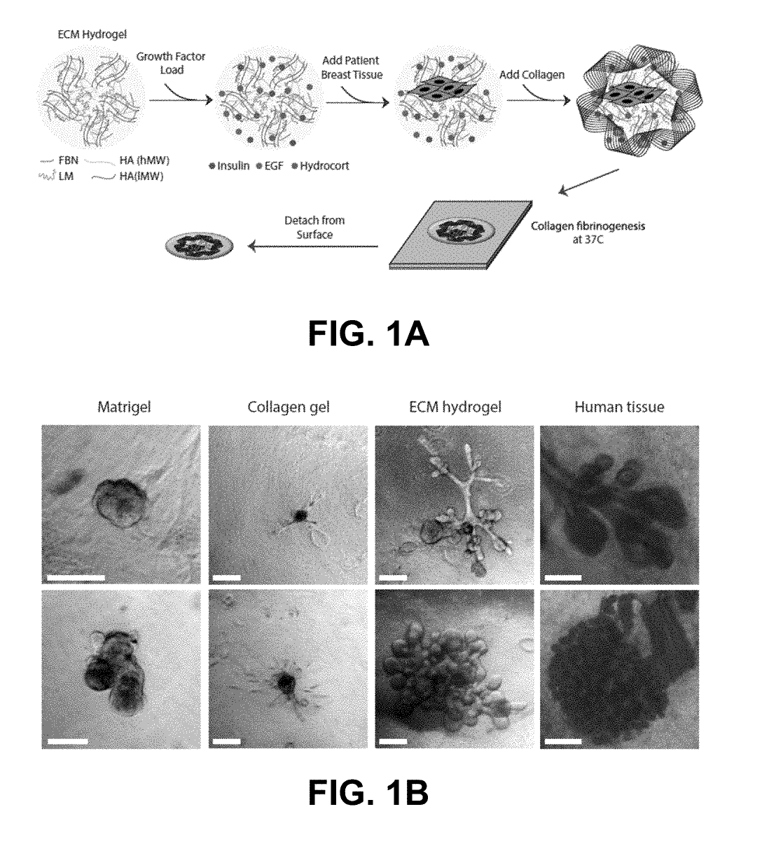

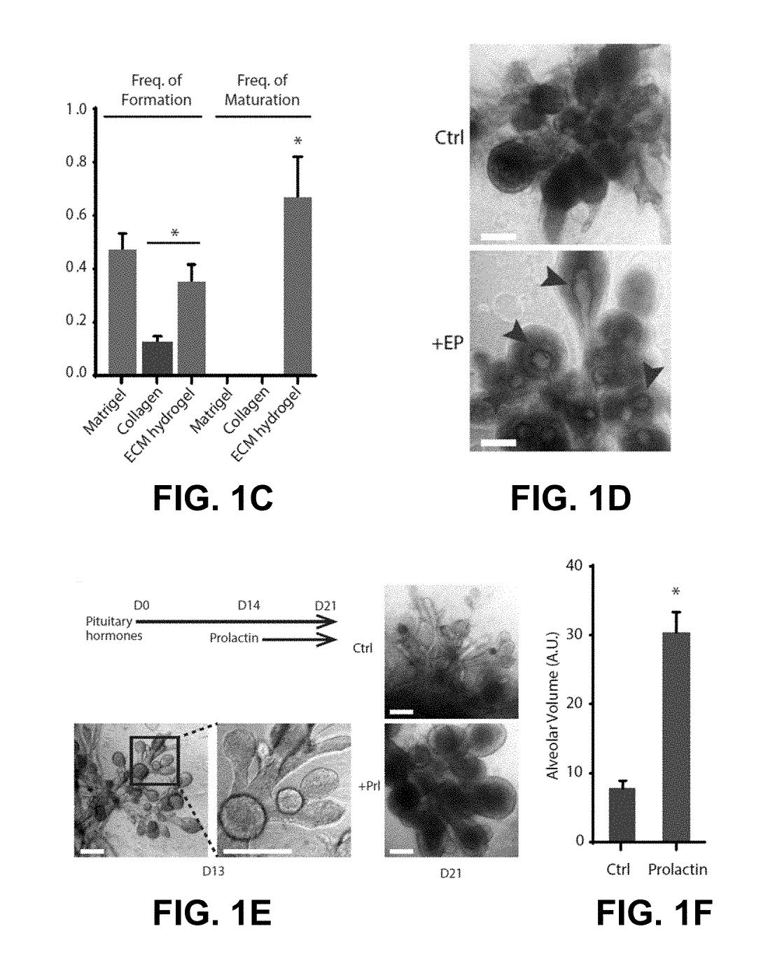

Using this approach, we established a three-dimensional hydrogel that was fabricated using the procedure depicted in FIG. 1A. These ECM hydrogels had several key features that were necessary to support breast tissue growth: (i) the hydrogels were fabricated with collagen, fibronectin and laminin, three ECM proteins present in human breast tissue in vivo (Schedin and Keely, 2011); (ii) the hydrogels incorporated hyaluronic acid, a glycosaminoglycan polysaccharide present in many human tissues, including the breast; (iii) the hydrogels were loaded with three growth factors during their fabrication—insulin, epidermal growth factor, and hydrocortisone—which have been shown to support the growth and differentiation of mammary epithelial cells (Hennighausen and Robinson, 2001; Mills and Topper, 1969; Yang et al., 1987); and (iv) after their creation, the hydrogels were detached from molds and cultured in suspension (Dhimolea et al., 2010; Emerman and Pitelka, 1977; Wozniak et al., 2003).

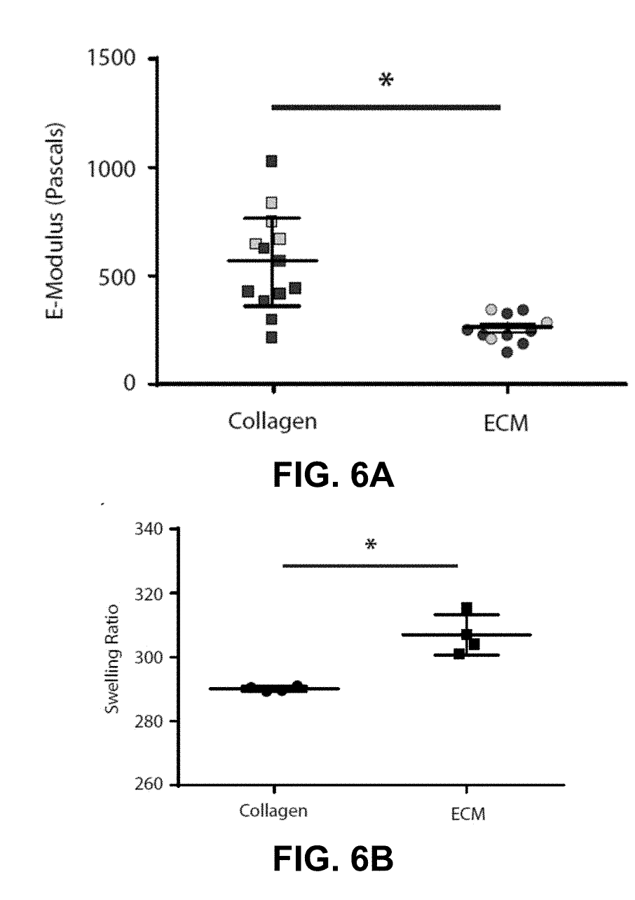

To understand the physical properties of these gels, we measured the swelling ratio and Young's modulus (elastic modulus) of collagen gels and our ECM hydrogels. Our ECM hydrogels exhibited a significantly larger swelling ratio than collagen gels (306.94+/−6.29 [mean+/−standard deviation] v 290.10+/−0.81 for collagen only gels; p<0.01), likely due to the presence of highly polar hyaluronans, which hydrogen bond extensively with water. Using atomic force microscopy (AFM), we found that collagen gels have an elastic modulus of 559.2 Pa+/−204.0, which was significantly larger than the elastic modulus of our ECM hydrogels, E=256.7 Pa+/−20.0 (p<0.05, FIG. 6A and FIG. 6B). The additional components in our ECM hydrogels, in addition to binding water, may partially disrupt collagen polymerization, resulting in a softer gel with increased water content; this would result in a smaller elastic modulus and higher swelling ratio. Importantly, the elastic modulus of our ECM hydrogels is in line with values previously reported for breast tissue in vivo (Paszek et al., 2005).

II. ECM Hydrogels Support the Growth of Complex Breast Tissues

When seeded into the ECM hydrogels, primary mammary epithelial cell clusters isolated from reduction mammoplasties rapidly grew into complex breast tissues with a seeding efficiency of 33%+/−6.3% (mean+/−SEM, FIG. 1B and FIG. 1C). The breast tissues that expanded in these hydrogels had complex ductal-lobular morphologies that closely resembled the epithelial structures present in the human breast (FIG. 1B, right). Breast tissue outgrowths with similar morphologies were observed from all of the patient samples that we assessed (7/7), indicating that the hydrogel scaffolds were consistently capable of expanding human breast tissue. In contrast, and consistent with prior findings (Yang et al., 1980), there was minimal or no outgrowth when primary mammary cells were seeded into polymerized collagen that lacked additional ECM components, or into basement membranes (Matrigel) with or without additional ECM components (FIG. 1B, FIG. 1C and FIG. \). The few outgrowths that did form in Matrigel were spherical with some ruffling at the edges, while the outgrowths that formed in collagen alone were primarily either thin ducts or spheres.

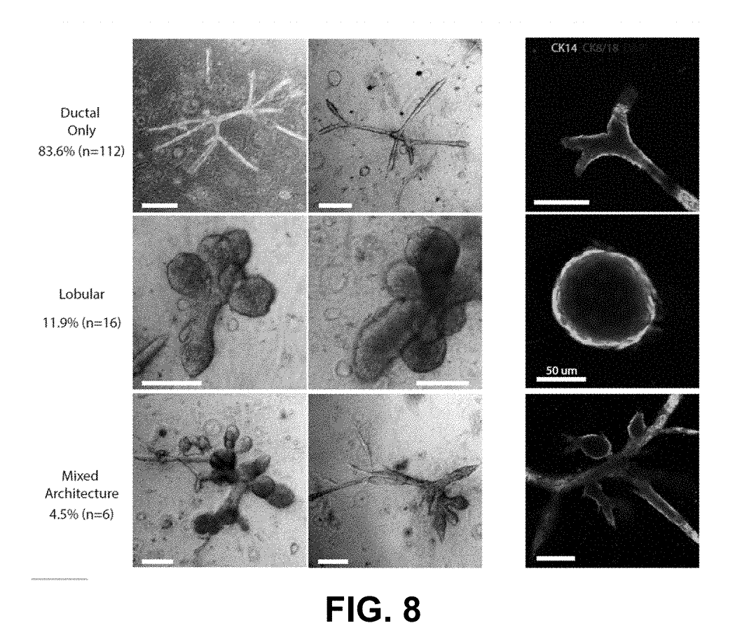

When single primary epithelial cells were seeded into the hydrogels, the tissue structure formation efficiency was much lower (0.16%) than that observed with primary cell clusters (33%). Moreover, only 4.5% of the tissue structures derived from single cells exhibited the complex mixed ductal-lobular morphologies that were exhibited in the majority of tissue structures derived from primary cell clusters (67%); thus, on an absolute scale, 0.0075% of single cells gave rise to tissue structures with complex ductal-lobular morphologies, whereas 26% of primary cell clusters gave rise to tissue structures with complex morphologies (FIG. 8). The remainder of structures formed from single cells were primarily thin, ductal (83.6%) or simple, lobular (11.9%) tissue structures. Importantly, even single cell-derived tissue structures with complex morphologies only stained positively for the basal marker CK14, and did not contain cells expressing the luminal marker CK8/18 (FIG. 8). These observations indicated that while single cells can form topologically complex structures with low frequency, the resulting structures fail to recapitulate the cell type complexity found in the mammary gland and in the tissue structures that were derived from primary cell clusters. Given these findings, we focused our further experiments on growing tissues from primary cell clusters.

III. Tissue Structures Exhibit Morphological Response to Hormones

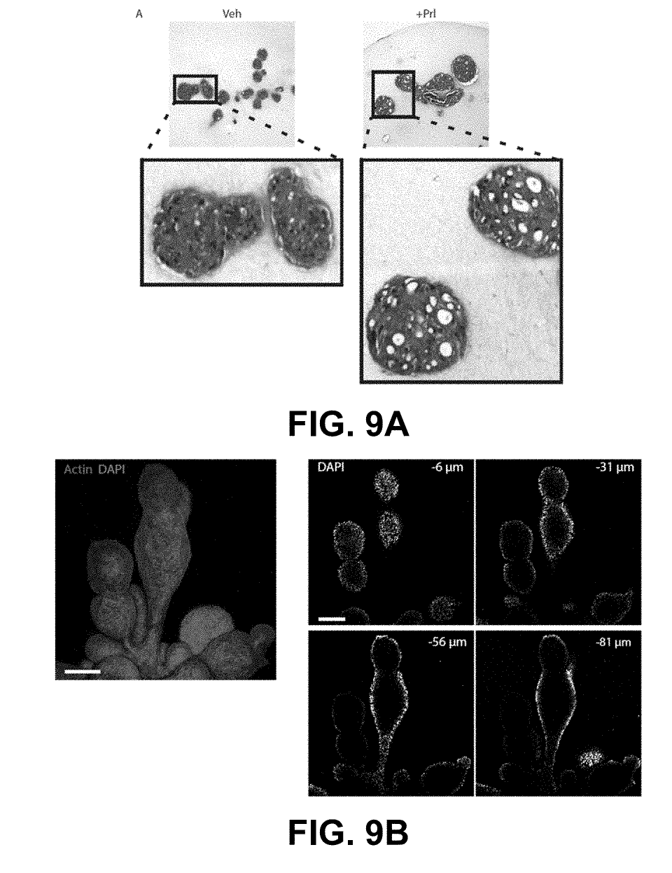

We next assessed if breast tissues cultured in hydrogel scaffolds respond to steroid, pituitary, or lactogenic hormones, which are known to stimulate the development of mammary epithelial tissue in vivo. Treatment of the ECM hydrogels with estrogen and progesterone stimulated mammary tissue structures to hollow, resulting in the formation of ducts and lobules with evident lumens (FIG. 1D, FIG. 9A and FIG. 9B); this observation suggested that these steroid hormones were promoting tissue structure maturation. When the hydrogels were supplemented with pituitary gland extracts, which contain several hormones important for mammary development, such as growth hormone, fibroblast growth factors, and follicle stimulating hormone (Hadden et al., 1989; Perez-Castro et al., 2012), there was a significant increase in both secondary and tertiary ductal branching of the expanded breast tissues (FIG. 1E). Treatment with prolactin further stimulated lobular expansion and caused a 4-fold increase in lobular volume accompanied by the formation of large lipid droplets, visible upon hematoxylin and eosin (H&E) staining (FIG. 1E, FIG. 1F, FIG. 9A and FIG. 9B).

IV. Kinetics of Tissue Growth and Maturation in Hydrogels

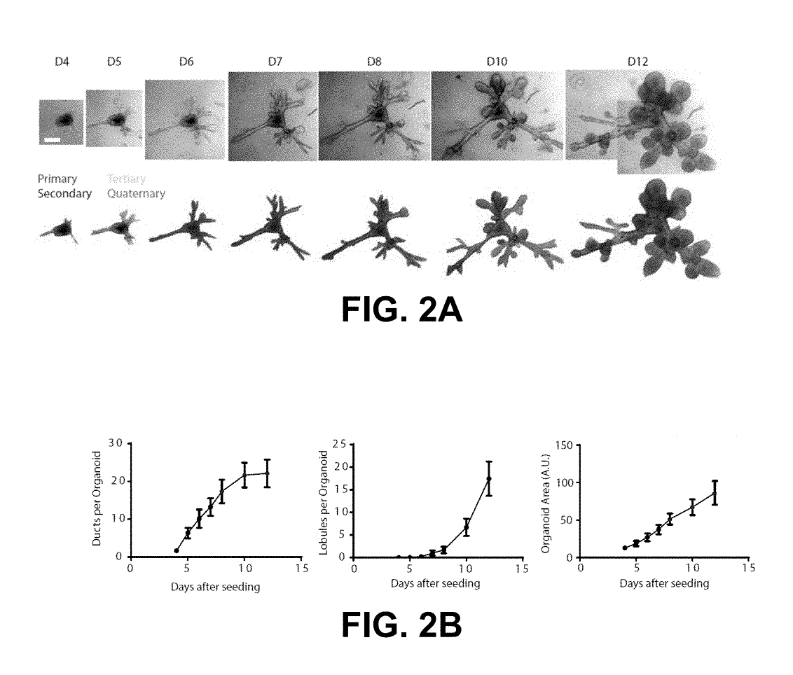

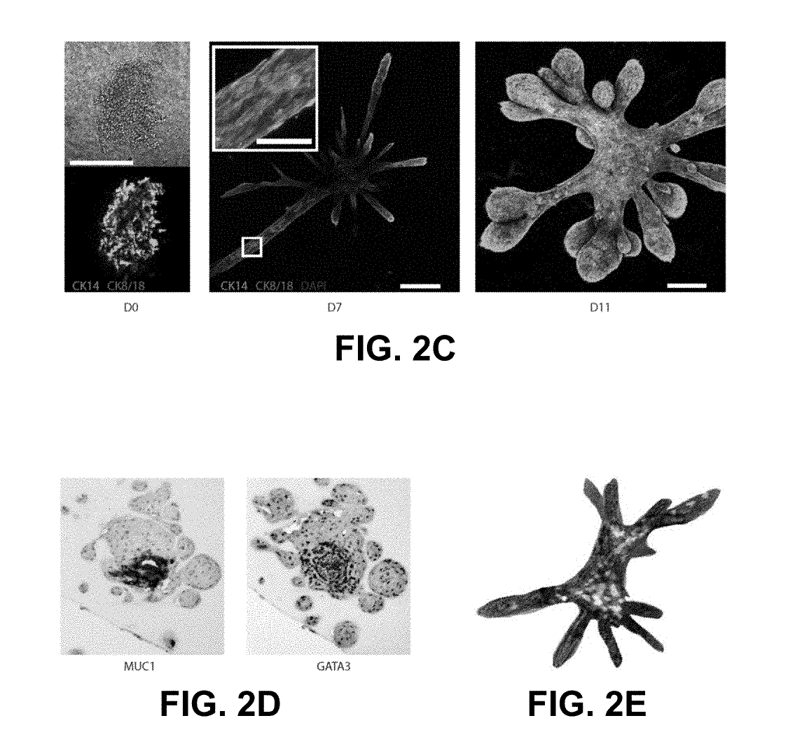

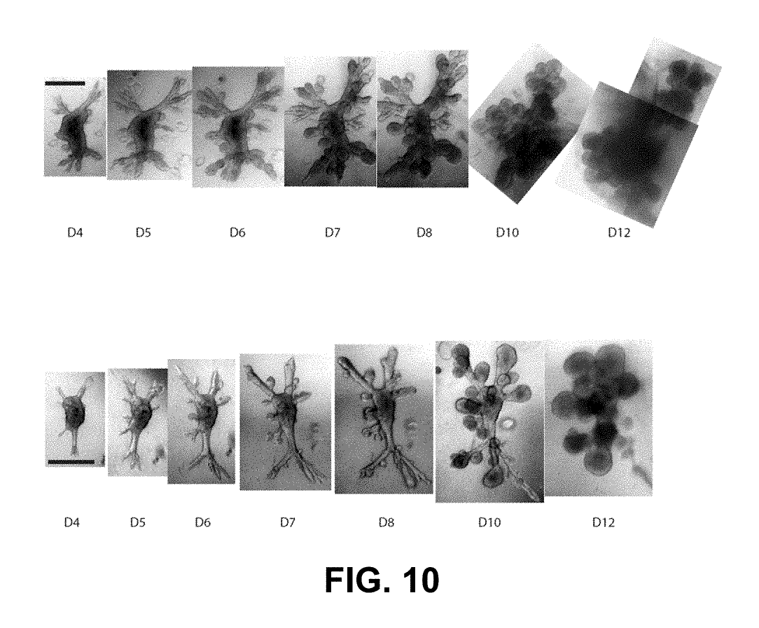

To examine the kinetics with which these structures matured, we captured bright-field images of structures over a span of 8 days, beginning at 4 days after seeding, the earliest time point at which we observed ductal outgrowths (FIG. 2A and FIG. 10). Analysis of these images revealed that the tissue growths had already sprouted primary ducts by 4 days, which gave rise to secondary and tertiary ducts over the next week. These secondary and tertiary ducts arose either through bifurcation of elongating ducts, or through side-branches that sprouted from ducts. After 8-12 days of tissue growth, there was a rapid increase in the number and size of lobules (FIG. 2A and FIG. 2B).

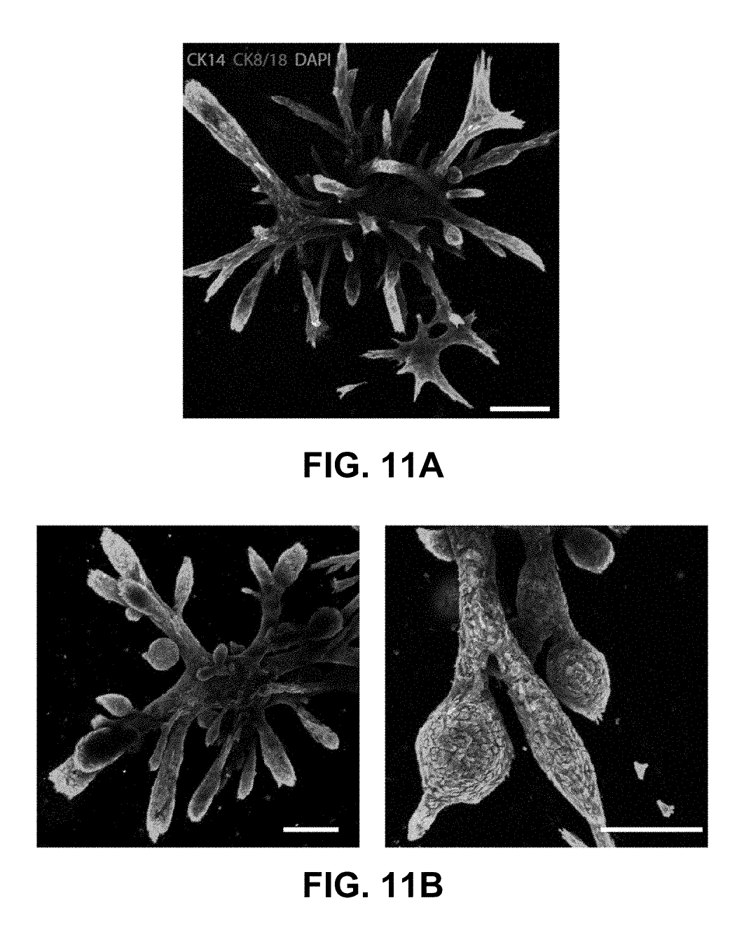

The primary cells seeded into hydrogels were initially disorganized clusters with intermixed basal (CK14⁺) and luminal (CK8/18⁺) cells (FIG. 2C). However, by 7 days, the cells had self-organized into an outer CK14⁺ basal layer with some CK8/18⁺ luminal cells in the interior of the expanding tissues (FIG. 2C center and FIG. 11A). At this early time point, the majority of newly initiated ducts were small and exclusively composed of CK14⁺ basal cells. However, as the outgrowths expand and mature, CK8/18⁺ luminal cells can be seen lining their interior (FIG. 2C right, FIG. 11A and FIG. 11B). In all patients, at least 60% of the mature tissue structure contained distinct luminal and basal layers.

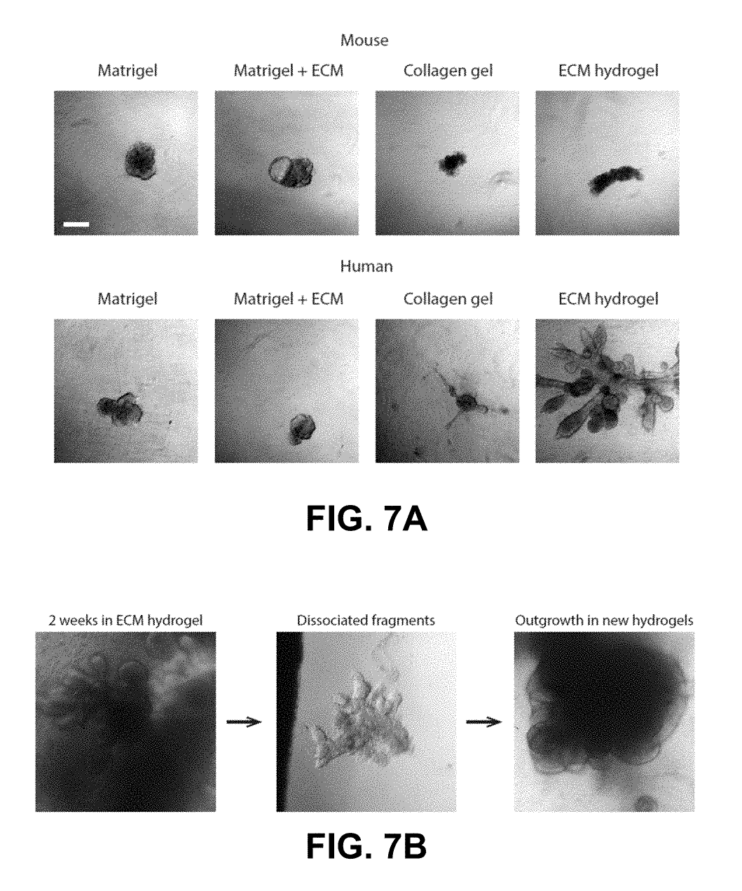

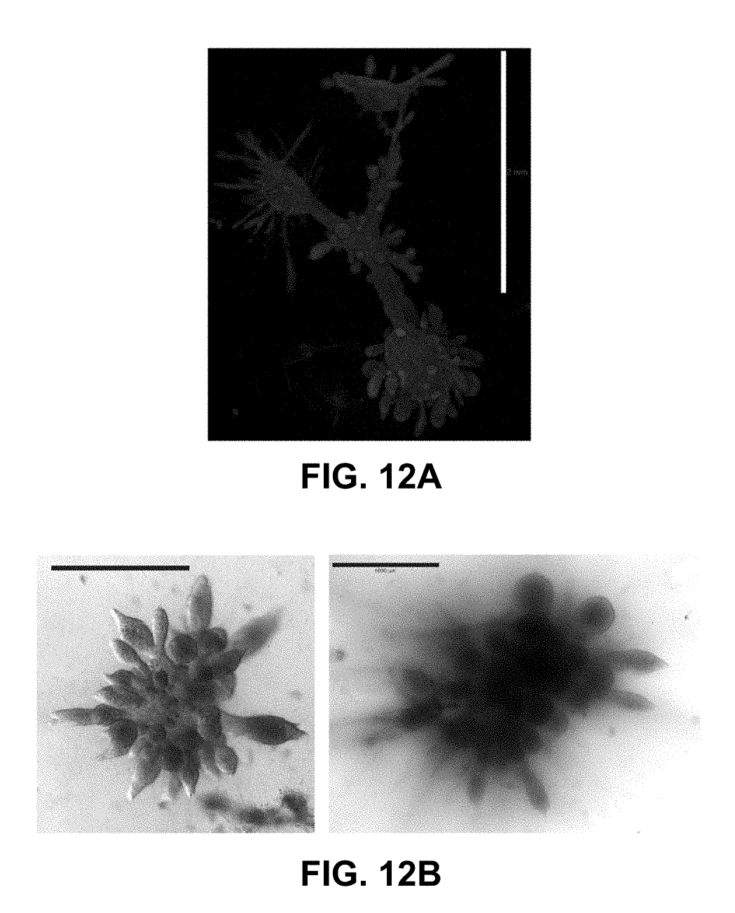

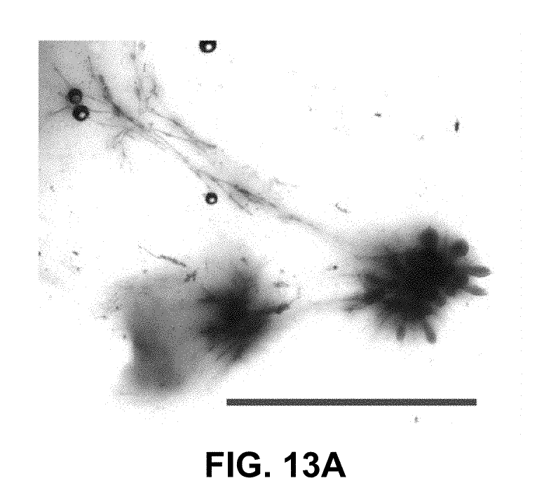

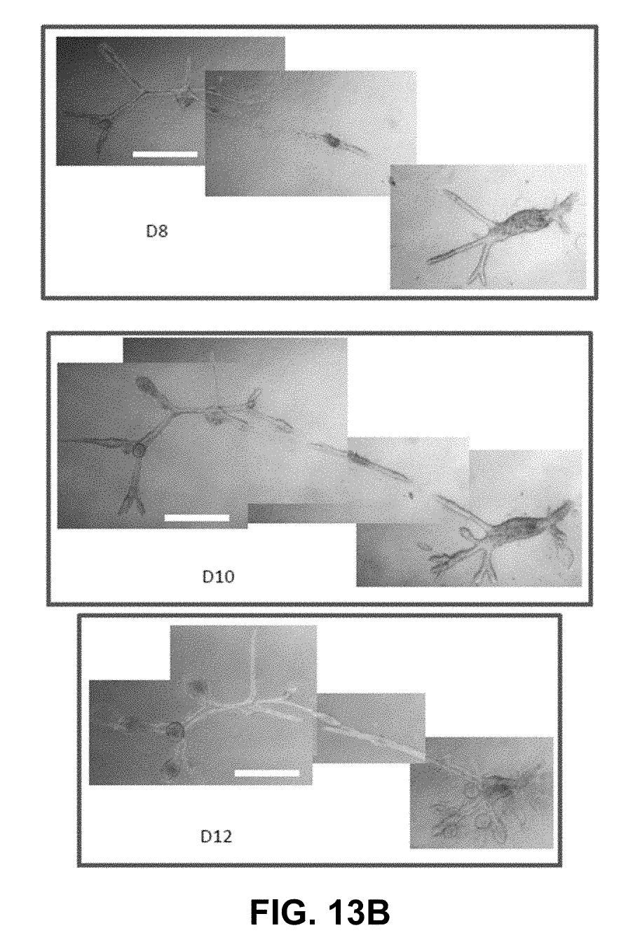

By 14 days, there was clear evidence of tissue maturation, with the lobule interiors staining strongly for both the luminal lineage marker GATA3, and the luminal differentiation marker, MUC1 (FIG. 2D); at this time, some of the lobule interiors also showed evidence of cavitation (FIG. 2D). Fully mature structures expanded to sizes of up to 3 mm in diameter (FIG. 12A and FIG. 12B), and remained viable for at least 8 weeks in culture in the same hydrogel. During this time, the developing and expanding tissues radically remodeled and condensed the hydrogels in which they were cultured, with evidence of this condensation up to 2 mm away (FIG. 2A, FIG. 11A, FIG. 11B, FIG. 13A and FIG. 13B). After 3-6 weeks, the tissue structures fully expanded to the size of the condensed pad and were unable to grow further. However, these structures could be removed from the hydrogels by enzymatic digestion and reseeded into new hydrogels, which support their continued growth (FIG. 7B).

Prior studies of the morphogenesis of mouse mammary tissue structures have indicated that the process of ductal initiation and elongation involves a dynamic reorganization of cells within 3D cultures (Ewald et al., 2008). To assess if this was also occurring in our primary human tissue structures, we stably labeled the primary cell clusters with fluorescent proteins before seeding them into the hydrogel scaffolds. Because the fluorescent proteins were delivered by lentivirus at a low multiplicity of infection, it was possible to assess the contributions of individual clones and their progeny to the formed mammary tissues. Using this approach, we found that the progeny of individual clones were dispersed throughout the tissue structures, rather than being localized to clonal patches (FIG. 2E). This suggested that cells underwent dynamic rearrangements as they proliferated to grow tissues. Time-lapse movies also showed dynamic rearrangements: mass cell migrations could be seen in the tissue structure cores, along ducts, and also within terminal ductal-lobular units (TDLUs).

V. Mammary Stem Cell Behavior in Ductal Initiation and Maturation

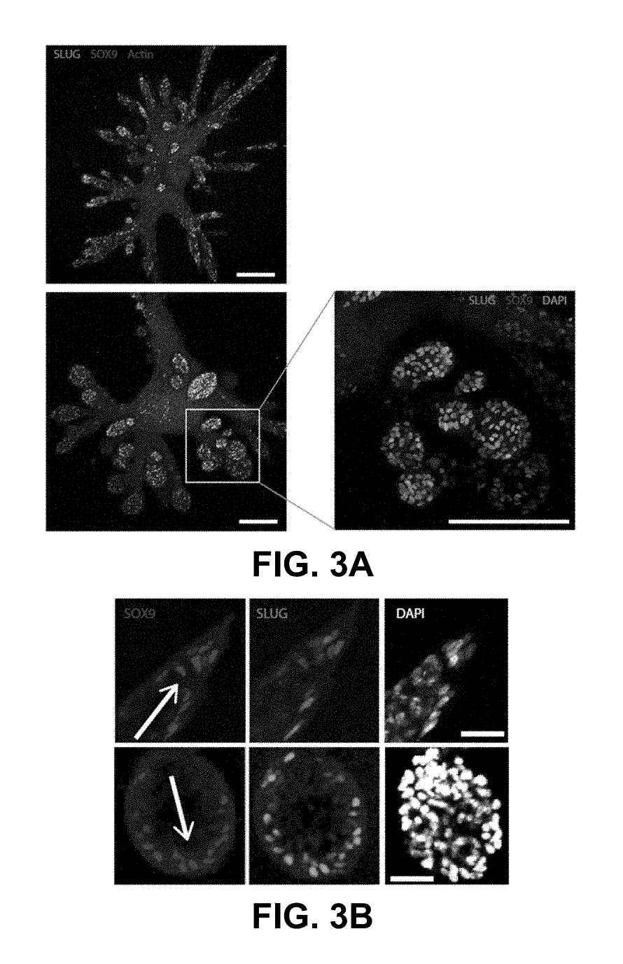

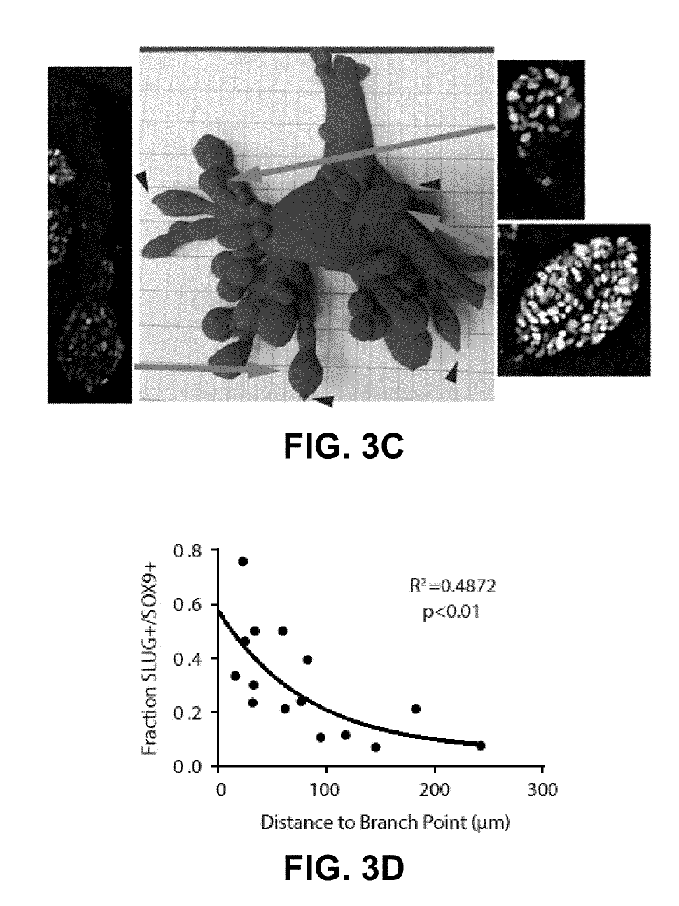

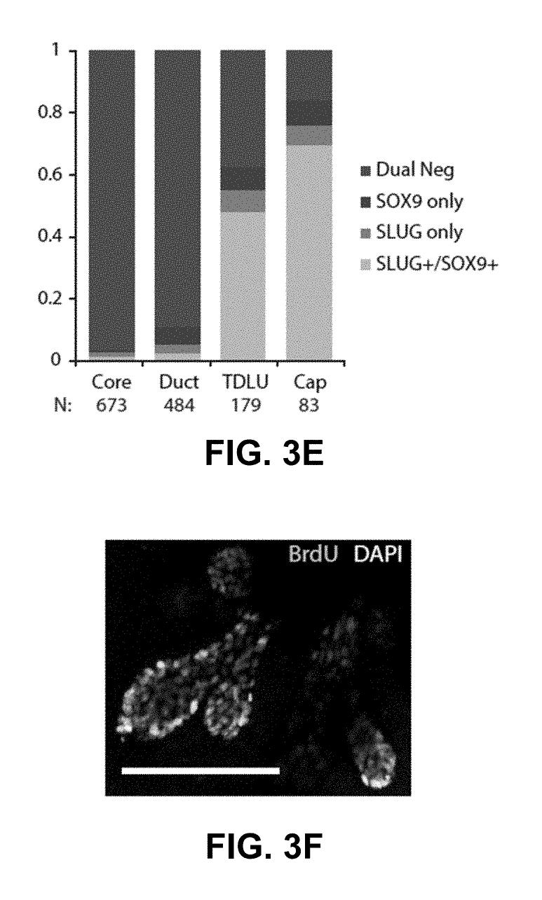

A unique strength of our three-dimensional hydrogel system is the ability to observe the behavior of mammary stem cells (MaSCs) and their contribution to the initiation and maturation of structural outgrowths. To identify putative MaSCs, we performed IF staining against the transcription factors SLUG and SOX9, which, when co-expressed, mark MaSCs in the murine mammary gland (Guo et al., 2012). SLUG⁺/SOX9⁺ cells were rarely seen within the core and ducts of tissue structures, but made up roughly half of the cells in the TDLUs. These TDLUs were typically 5-8 cells thick, and the layer of cells in direct contact with the ECM (termed the “cap” region) was most enriched for the dual-positive cells, with roughly two-thirds of cells co-expressing SLUG and SOX9 (FIG. 3A and FIG. 3E). In both ducts and lobules, the dual-positive cells were enriched in the cap region of the expanding outgrowth, in direct contact with the extracellular matrix, suggesting that this contact could be involved in maintaining stem cells in an undifferentiated state (FIG. 3B and FIG. 3E).

To assess the topological properties of these tissue structures, we rendered a surface model from three-dimensional confocal microscopy images and used a Dimension Elite 3D printer to fabricate a high-resolution 1500× scale physical model of an tissue structure stained for filamentous actin (FIG. 3C). Examination of this physical model revealed that the outgrowths containing the highest fraction of SLUG⁺/SOX9⁺ cells were also the shortest: when side-branches started to form, nearly all of the cells were dual-positive, but, as the ducts elongated, there was a gradual decrease in the fraction of dual-positive cells (FIG. 3D). This suggested that side-branches were initiated by the proliferation of SLUG⁺/SOX9⁺ cells, which subsequently differentiated to give rise to interior cells, concurrent with ductal elongation.

VI. SLUG⁺/SOX9⁺ Leader Cells Direct Ductal Elongation

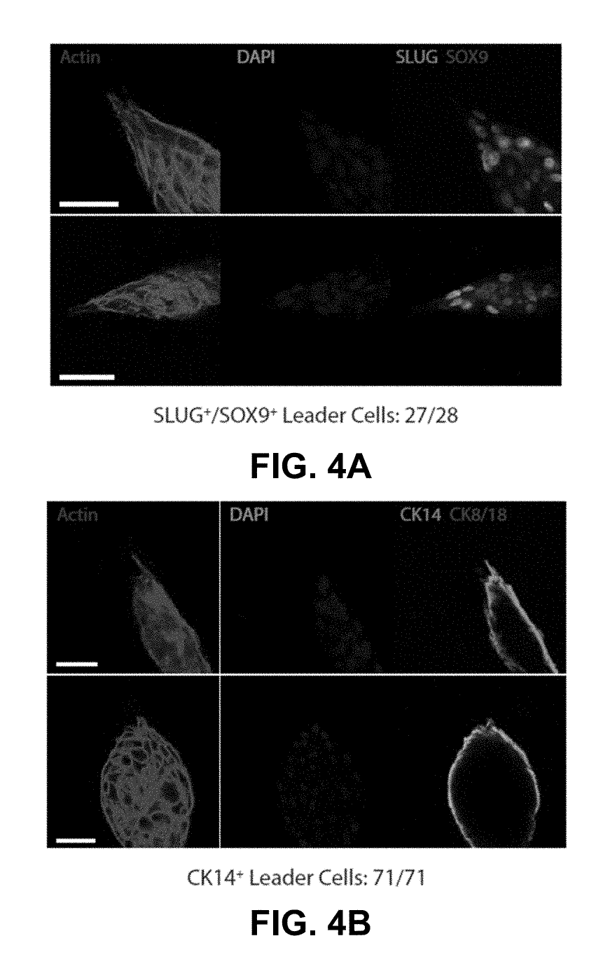

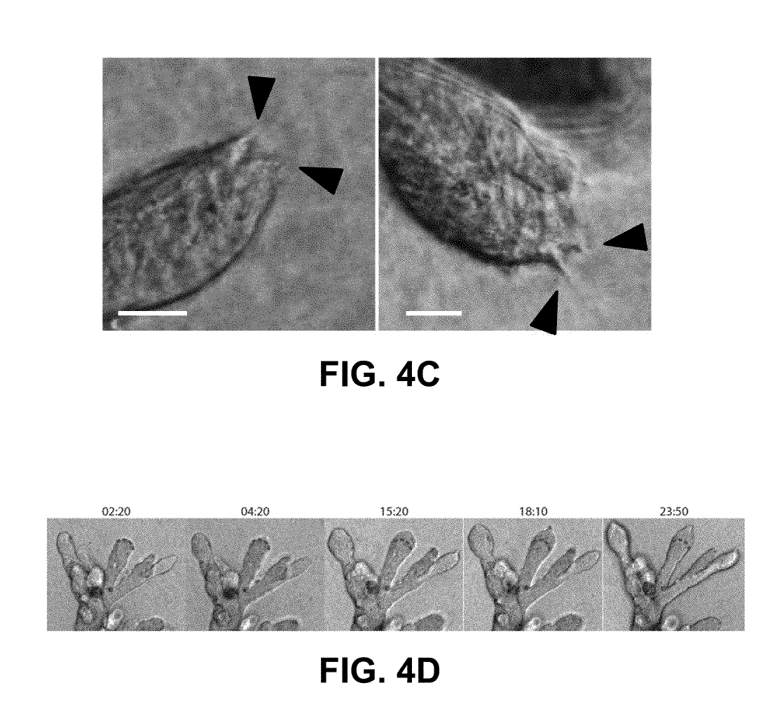

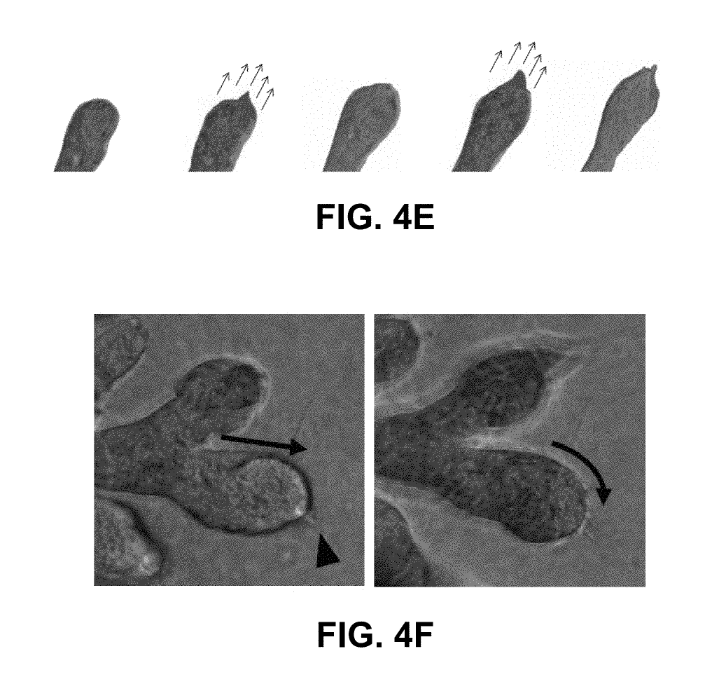

Examination of the printed 3D model also revealed the presence of small tips at the leading edges of elongating ducts. Confocal microscopy showed that these tips contained one or two leader cells that were polarized in the direction of ductal elongation. The leader cells stained positively for filamentous actin and protruded from the structures in the direction of ductal elongation (FIG. 4A and FIG. 4B). The leader cells expressed basal cytokeratins (FIG. 4B) and co-expressed SLUG and SOX9 (FIG. 4A). While the majority of outgrowths contained one leader cell, occasionally outgrowths contained multiple leader cells in different orientations (FIG. 4C).

Time-lapse microscopy provided additional insights into the relationship between these leader cells and ductal elongation. Ductal elongation was always preceded by a transient extension of leader cells that physically engaged with and deformed the extracellular matrix (FIG. 4D and FIG. 4E,). At times, the force of this interaction between leader cells and the matrix caused them to break away from the ducts and become isolated in the matrix. The direction in which the leader cells extended was always the direction of the next wave of ductal elongation. When the direction in which the leader cells emanated was different from the previous direction of elongation, the ducts re-oriented in the new direction specified by the leader cells prior to the next wave of elongation (FIG. 4F). This ductal re-orientation appeared to be induced by the collective rotation of cells in the lobule, which occurred prior to ductal elongation. After the ducts re-oriented, they elongated for a period of time, after which the elongation ceased. After ductal elongation ceased, new leader cells emanated from the ductal tips to initiate the next cycle of elongation.

Previous studies of murine mammary organogenesis, performed in Matrigel, have indicated that ductal elongation is not driven by leader cells, but rather through the collective expansion and migration of luminal cells (Ewald et al., 2008). Consistent with that study, when mammary tissue fragments from C57BL/6J mice were seeded into our ECM hydrogels, they grew and ruffled as previously described, but did not exhibit any leader cell activity (FIG. 7A and FIG. 7B). These findings suggest that leader cells may play different roles in human and mouse mammary morphogenesis.

### DISCUSSION

We have described ECM hydrogels with defined components that support the growth and differentiation of mammary tissues from patient-derived cells. The tissues that form in these hydrogels consist of multiple cell lineages and respond to steroid, pituitary, and lactogenic hormones. While stromal cells are essential for making ECM, our findings indicate that their active participation is not required for human mammary morphogenesis. Although somewhat unexpected given the instructive role that stromal cells appear to play in mammary development (Wiseman and Werb, 2002), this finding is consistent with observations in other organoid systems. For example, intestinal epithelial cells self-organize into intestinal crypts when placed into basement membrane cultures (Sato et al., 2009), lingual epithelial cells recapitulate the complex organization of tongue epithelium (Hisha et al., 2013), and neuro-ectodermal cells self-organize into cerebral organoids that recapitulate key aspects of brain development (Lancaster et al., 2013). An emerging theme from these studies is that epithelial cells have an inherent ability to self-organize into complex tissues without the support of stromal cells, provided they are placed into suitable 3D culture conditions.

Because our breast tissues were cultured in transparent hydrogels, we were able to directly observe the processes of ductal initiation, elongation, and branching. We observed two main methods of branching: (i) bifurcation at the ends of ducts and (ii) ductal side branching, both previously seen in mouse mammary morphogenesis (Fata et al., 2004; Lu and Werb, 2008). Interestingly, we found that ductal budding and elongation in the primary human tissues was driven by SLUG+/SOX9+ leader cells that express filamentous actin and basal cytokeratins (CK14+). Leader cells do not appear to play a role in ductal elongation in mouse mammary organoids, which is instead driven by the mass action of luminal cell layers (Ewald et al., 2008). However, leader cells with filamentous actin-positive protrusions have been implicated in ductal elongation in the air sacs of flies (Cabernard and Affolter, 2005; Lu and Werb, 2008) and in vascular endothelia (Gerhardt et al., 2003). Taken together with previous studies (Ewald et al., 2008), our findings suggest that different species may use very different mechanisms to promote mammary morphogenesis.

We were able to identify where putative human mammary stem cells were localized by staining for SLUG and SOX9, which label MaSCs in mice. Cells that were dual-positive for these markers were localized primarily to the cap regions of new outgrowths, and were in direct contact with the ECM. This finding raises the possibility that ECM contact may be necessary to maintain stem cells in an undifferentiated state. This is consistent with the role of ECM in regulating stem cell self-renewal in the hematopoietic system, hair follicles, and the brain (Gattazzo et al., 2014).

The localization of stem cells to the tips of developing lobules is consistent with recent findings in the human breast (Honeth et al., 2015). By sectioning and staining primary human tissue, Honeth et al. found that MaSCs were enriched at the tips of immature lobules, with decreased MaSC numbers in larger and more mature lobules. The possibility that MaSCs may be localized to the cap region of end buds has also been proposed for the murine mammary gland (Smalley and Ashworth, 2003; Srinivasan et al., 2003).

We found that the SLUG+/SOX9+ leader cells are motile, and express the basal cytokeratin, CK14. These findings are consistent with prior studies demonstrating that MaSCs are found in the basal cell compartment (Rios et al., 2014; Shackleton et al., 2006; Stingl et al., 2006), as well as reports that induction of mammary cells into a stem-like state results in the upregulation of basal markers and an onset of motility (Mani et al., 2008). This raises the possibility that the cells in, or induced into, a stem cell state are simultaneously capable of self-renewal and capable of maneuvering through and engaging the ECM. These programs could be co-opted by cancer cells, where the properties of self-renewal (allowing for continued proliferative potential) and motility through the ECM (allowing for dissemination and expansion) might be selected for. We anticipate that the ability to grow hormone-responsive human breast tissue in hydrogels with defined components will empower future studies of human mammary gland development and biology, with potential implications for our understanding of breast cancer biology. The presently disclosed three-dimensional hydrogels can also be used to identify mechanisms of drug resistance in a patient's own cells.

### Supporting Methods

I. Preparation of Primary Tissue

Elective reduction mammoplasty patient tissue samples were obtained from the Maine Medical Center Biobank. Mouse mammary tissue was collected from 12-week old C57BL/6 mice. Tissues were mechanically dissociated using a sterile razor blade into approximately 3-5 mm³ fragments, and resuspended in Dissociation Buffer (MEGM (Lonza) containing 3 mg/mL collagenase (Roche), 250 units/mL hyaluronidase (Sigma Aldrich), 1× antibacterial-antimycotic (Gibco)) at a concentration of 0.2 gm/mL, and incubated with rocking at 37 C overnight. Tissue structures were allowed to pellet by gravity for five minutes, and were washed five times in PBS containing 5% FBS (Sigma), in order to remove any associated stromal cells. Prior to seeding into hydrogels, further fibroblast depletion was carried out by plating tissue structures in DMEM containing 10% FBS on tissue culture treated dishes for 90 minutes. Tissue structures were then washed in PBS and resuspended in culture media.

II. Preparation of Hydrogels

Hydrogels were composed of 1.7 mg/mL rat tail collagen (EMD Millipore), 10 μg/mL hyaluronan 150 kDa (Sigma Aldrich), 10 μg/mL hyaluronan 500 kDA (Sigma Aldrich), 40 μg/mL laminin (Life Technologies), and 20 μg/mL fibronectin (Life Technologies), supplemented with 0.05% insulin, 0.05% hydrocortisone, and 0.05% epidermal growth factor (Lonza CC-4021G, CC-4031G, and CC-4017G respectively). Collagen was pH neutralized by adding 0.125 volumes of 0.1 N NaOH on ice, diluted to a final concentration of 1.7 mg/mL in MEBM media (Lonza), followed by the addition of the remainder of components. Next tissue structures, resuspended in the appropriate culture media (e.g., MEGM, e.g., MEBM supplemented with growth factors, e.g., EGF, insulin, hydrocortisone, etc.), were added to the solution. The hydrogels were polymerized in 4-chamber slides (Corning) at 37° C. and 5% CO2 for 1 hr, at which point culture media (e.g., MEGM) was added and the gels were detached from the slide with a pipette tip.

In other experiments, breast tissues have been successfully grown in 3D hydrogels in a variety of culture media, including:

1. MEGM (Lonza CC-3151 & CC-4136) with cholera toxin added (100 ng/mL)

3. MEGM (Lonza CC-3151 & CC-4136) with no BPE added

4. FAD2 media (Fridriksdottir et al, Nature Communications 6, Article number: 8786 (2015)): 75% DMEM, high glucose, no calcium (Life Technologies), 25% Ham's F12 Nutrient Mixture (F12, Life Technologies) with 2 mM glutamine, 0.5 μg/mL hydrocortisone, 5 μg/mL insulin, 10 ng/mL cholera toxin (Sigma-Aldrich), 10 ng/mL EGF (Peprotech), 1.8×10-4 M adenine, 10 μM Y-27632 (Y0503, Sigma-Aldrich) and 5% FBS (Sigma-Aldrich). After 2 days of culture, 10 μM, 54317 (Sigma-Aldrich) and 50 μM RepSox (R0158, Sigma-Aldrich) was added.

5. FAD2 media without the addition of 54317 and RepSox

6. Sequential combinations of MEGM and FAD2 media. 2 days of culture in FAD2 followed by switching to MEGM, 4 days in FAD2 followed by MEGM, 2 and 4 days in MEGM followed by switching FAD2

7. FAD2 and MEGM at various ratios (1:1, 1:2, 1:9)

III. Lentiviral Production Lentivirus production was performed as previously described (Gupta et al., 2005). LeGO lentiviral vectors were kindly provided by Kristoffer Riecken (Weber et al, 2011). Virus was produced from three separate vectors encoding mCherry, Venus, and Cerulean fluorescent proteins.

IV. Immunofluorescence/Immunohistochemistry

Samples were fixed with 4% paraformaldehyde for 30 minutes at room temperature. Pads were permeabilized overnight using 0.1% TritonX-100 and incubated with blocking solution (PB ST with 3% goat serum and 3% BSA) for 2 hr at room temperature and stained with the appropriate primary antibody in blocking buffer overnight at 4° C. The samples were washed with PBS, and incubated with an Alexa Fluor-labeled secondary antibody (Cell Signaling) and phalloidin-647 (Life Technologies). Samples were washed, stained with 1 ug/ml DAPI.

BrdU (Sigma Aldrich) was added at 10 μM for 2 hr, after which samples were washed with PBS and fixed with 4% paraformaldehyde. Anti-BrdU antibody was purchased from Cell Signaling Technologies and staining was performed according to manufacturer protocol.

IHC embedding, sectioning, and staining was performed at the Koch Institute Histology Core Facility. Samples were fixed in 4% neutral buffered formalin overnight, washed with 70% ethanol, and paraffin embedded. IHC was performed using the ThermoScientific IHC Autostainer 360.

Primary antibodies used in this study for IF were CK14 (Life Technologies; 1:300; RB-9020-P), CK8/18 (Vector; 1:500; VP-C407), SLUG (Cell Signaling; 1:400;C19G7), and SOX9 (Sigma; 1:50; WH0006662M2). IHC antibodies used were GATA3 (Cell Signaling; 1:6400; 5852), MUC1 (AbCAM; 1:100; 15481).

V. Microscopy

Immunofluorescence images were captured using a Zeiss LSM 700 and analyzed with LSM Viewer. IHC images were captured using Zeiss Axiophot. Time-lapse movies were captured using a Nikon TE2000 with a heated stage and 5% CO2.

VI. Physical Characterization of Hydrogels

The elastic modulus of the hydrogels was measured via Hertzian analysis of atomic force microscopy (AFM) force curves (Lin et al., 2007). Hydrogels were mounted on a glass slide and placed in the AFM (Veeco, Nanoscope IV with picoforce scanner head). The tip (Novascan, k=14 N/m functionalized with 45 μm polystyrene ball) was then brought into contact with the sample. Force displacement curves were obtained by monitoring the deflection of the tip throughout a z-displacement of 4 microns. Force exhibited by on the tip was calculated according to the equation:

F=k_{c}(d−dₒ)

where k_{c} is the spring constant of the cantilever (14 N/m), d−dₒ is the tip deflection following contact with the gel.

According to Hertzian analysis, the modulus may then be determined by the following equation, which accounts for the spherical geometry of the AFM tip:

$$E = \frac{F ⋆ 3 ( 1 - {v}^{2} )}{4 ⋆ \sqrt{R}} ⋆ \frac{1}{{( ( z - {z}_{0} ) - ( d - {d}_{0} ) )}^{3 / 2}}$$

where (z−zₒ) is the applied translation of the cantilever, r is the radius of the tip, and v is the Poisson's ratio (assumed to be 0.5 for an incompressible hydrogel). The elastic modulus was calculated using a linear regression of the force on the tip and the displacement relative to the gel, and then correcting for the geometric factors corresponding to tip geometry and Poisson's ratio.

Swelling ratios (SR) were calculated using the equation:

$$SR = \frac{{M}_{w} - {M}_{d}}{{M}_{d}}$$

where M_{w}=wet weight and M_{d}=dry weight.

### Example 2

### A Novel 3D Hydrogel Scaffold for Production of Human Milk and Testing Potential Lactogenic Therapies

### Introduction

There are major differences between human breast milk and formula, in terms of lipid, carbohydrate, and protein content, and pediatricians currently recommend that mothers breastfeed for the first year to maximize the future well-being of their children. Unfortunately, many women are unable to produce milk or have jobs that limit the how long they can afford to breastfeed. For such women, there are few alternatives to formula. The presently disclosed subject matter describes the first known method for producing human milk in laboratory cultures.

In addition to the women that are unable to breastfeed, a large number of breastfeeding women have difficulties producing enough milk for their infants. Some have estimated that up to 25% of women have milk volume difficulties, which can lead to either supplementing with formula, or, in many cases, a total cessation of breastfeeding and switch to exclusively formula feeding. As there is currently no FDA-approved treatment for milk underproduction, mothers often turn to herbal supplements that are purported to improve milk production. These supplements vary across cultures, and include herbs such as fenugreek and milk thistle. While these supplements have helped many women, their herbal nature and the lack of regulation may lead to unreliable efficacy and potential issues with contamination with undesirable agents. In addition to the use indicated above, the presently disclosed subject matter also provides a method for identifying the active agents in herbal and any other chemical mixtures that enhance milk production in women.

The presently disclosed subject matter in some aspects consists of a novel 3D hydrogel scaffold engineered as well as the sequence of hormonal and growth factor treatments that together support the growth of patient-derived human mammary tissues that are capable of producing human milk.

Use of the Hydrogel System to Find Compounds that Alter Milk Production

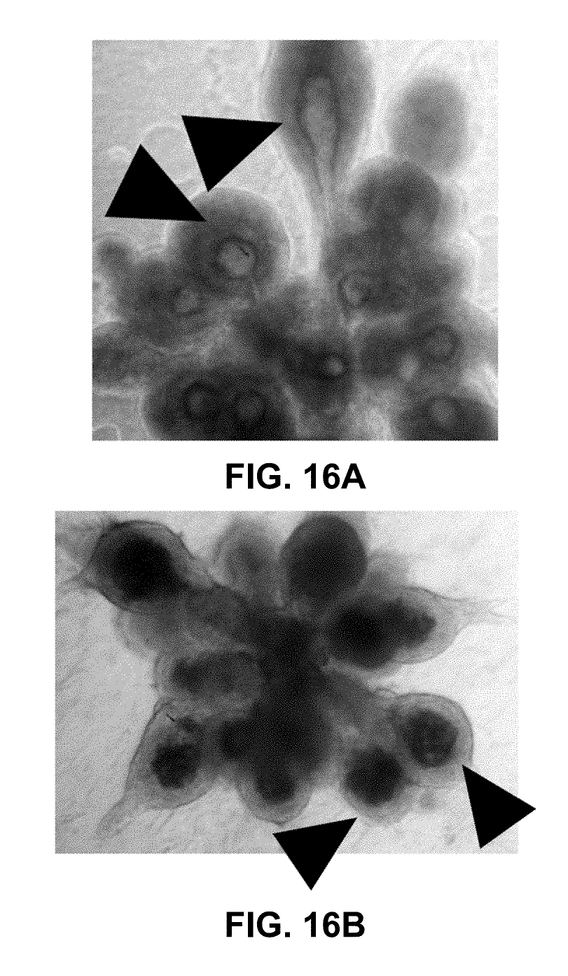

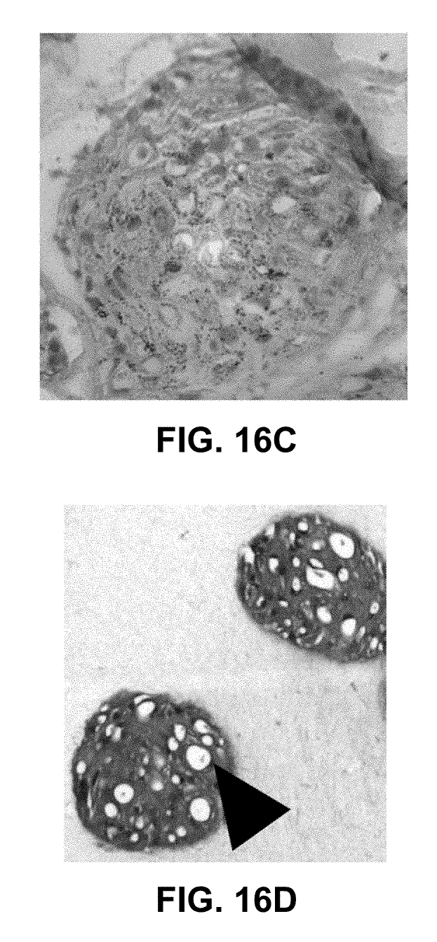

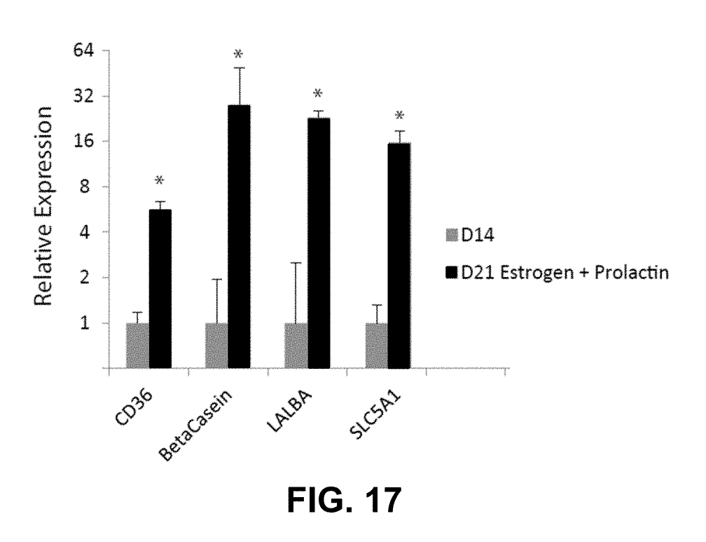

In some aspects, the presently disclosed subject matter provides a set of conditions that promote the production of milk from patient-derived mammary tissues grown in the hydrogel cultures. When grown in the presence of estrogen and progesterone for 2 weeks, the human tissues mature and hollow. Addition of prolactin promotes the formation of an opaque substance that fills the luminal space of the structures. This substance is lipid rich as indicated by oil red 0 staining and H&E staining (FIG. 16).

There are several quantifiable readouts that are currently established for quantifying milk production—opacity of the lobules, lobule size, oil red o intensity, lipid area in H&E stained tissue sections, and western blot analysis of milk proteins.

The presently disclosed subject matter can be used to assess the efficiency and active ingredients of agents that are suspected to increase milk production (termed galactogogues). As an example, fenugreek is a widely used herbal remedy to increase milk production, yet the active ingredient is not known. There are numerous side effects of fenugreek, particularly on blood glucose, that likely arise from the presence of multiple compounds in the herb. A use of the presently disclosed subject matter would be to chemically fractionate fenugreek and independently add the fractions to the mammary tissues expanded in our hydrogels, to determine which components of the herb alter milk production. The same approach can be taken for any other suspected galactogogues, or for the testing of novel chemicals for their ability to drive lactation.

### Example 3

### A Novel 3D Hydrogel Scaffold for Developing Personalized Therapies for Cancer Patients

### Introduction

For patients that present with cancer today, therapies are typically selected based on the tumor type and the extent to which it has invaded. For example, all patients with triple-negative breast tumors would receive the same therapy, and patients with ER-positive breast tumors would receive a different therapy. The problem with this approach is that tumors within the same type or class are not homogeneous, and often differ with respect to their genetic mutations, expression patterns, and response to therapy. Many researchers and clinicians have argued that patient outcomes could be significantly improved if cancer therapies were not selected based on the tumor type or class, but were tailored to the specific tumor presented by a specific patient. One approach currently being explored in clinical trials to ‘personalize’ therapy is to select drug combinations based on the pattern of mutations present in a patient's tumor; another approach being explored in trials is selecting therapies based on the gene-expression profile of a patient's tumor. While promising, these approaches to personalized medicine have not yet been proven to work, and there is good reason to believe that the mutations and expression patterns of cancers will be imperfect predictors of how the cancer cells in a patient will respond to any given drug.

The presently disclosed subject matter describes a different approach for identifying personalized therapies that are targeted to a patient's specific tumor. The approach described is to assess drug sensitivities using a patient's own cancer cells in culture, and to use this approach to develop a personalized cocktail of drugs to treat his or her specific tumor. To date, this approach has not been feasible because it has not been possible to grow cancer cells from patient tumors in the lab. The presently disclosed subject matter includes a novel 3D hydrogel scaffold engineered to resolve this problem, whose properties are described in the text and figures herein. Using these hydrogels, cancer cells can be grown from a patient's tumors and one can assess drug sensitivities within 1-4 weeks. With this rapid timeframe, the presently disclosed subject matter can be used to select a cocktail of drugs that is personalized to a patient's particular tumor.

### Use of the Hydrogels for Chemical Screening

The major limitation to screening a patient's particular cancer cells for drug sensitivities is that it is currently not possible to expand cancer cells in culture from patient tumors in a reasonable timeframe. Current methods take many months—ranging from 2-9 months—to expand cancer cells from patient tumors, and the cells that expand after this time are invariably rare clones that have adapted to the culture conditions, and do not reflect the morphology, mutations, cell-surface marker profiles, or drug sensitivities of the cancer cells in the patient's original tumors.

Our presently disclosed hydrogel resolves this barrier by enabling the expansion of a patient's cancer cells within a short timespan of 1-2 weeks. This makes it possible, for the first time, to systematically screen a patient's cancer cells in the lab for drug sensitivity, and use this information to inform and personalize the cocktail of drugs that are combined for this patient's therapy.

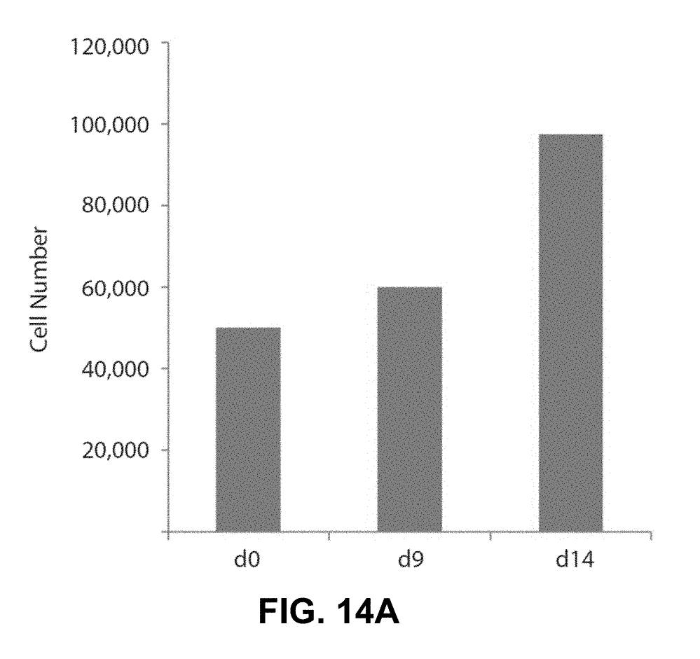

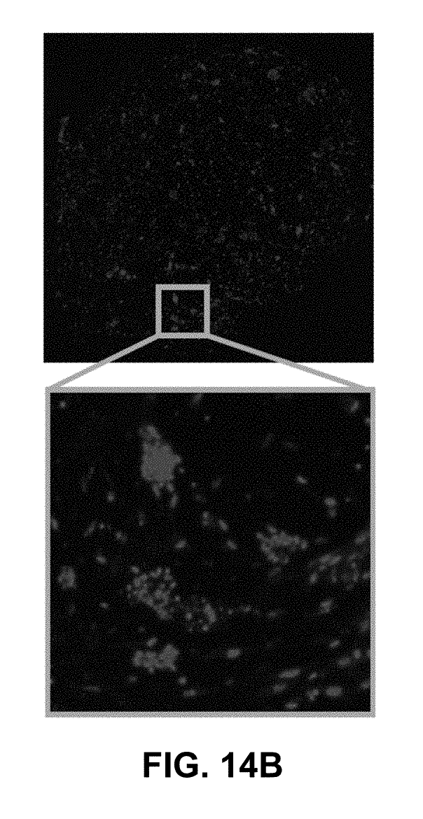

As an illustration of the presently disclosed subject matter, in FIG. 14A, we show a 2-fold expansion of a patient's tumor cells after just 2 weeks in culture.

As another illustration of the presently disclosed subject matter, in FIG. 14B we show a confocal microscopy image of cancer cells expanded in these hydrogels for 1 week, and stained with the live-cell dye DRAQ5. In the image, one can clearly see both clumps of cancer cells in spheroids, as well as the invasion of cancer cells emanating from the spheroids in cords, and also many single cancer cells that are scattered through the plane of the hydrogel. Remarkably, this unique pattern of growth was noted in the pathology report, generated several weeks after obtaining these results. It is also worth noting that this particular tumor is ER/PR positive, and that such tumors are notoriously difficult to establish in culture or in primary xenografts.

The data in FIG. 14A and FIG. 14B indicate that the majority of the cancer cells in the patient's tumor sample must have successfully proliferated in our hydrogel culture.

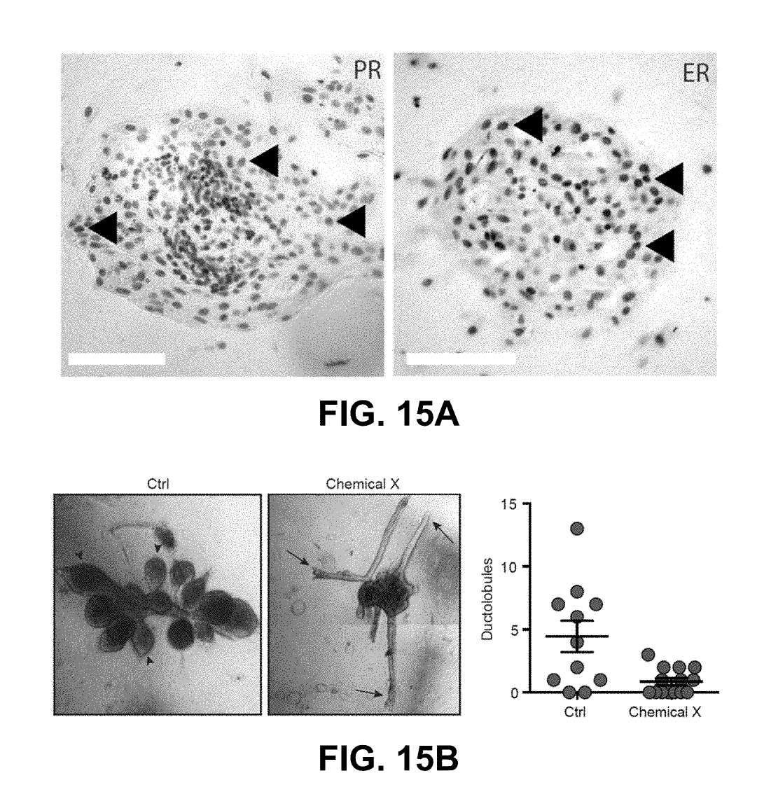

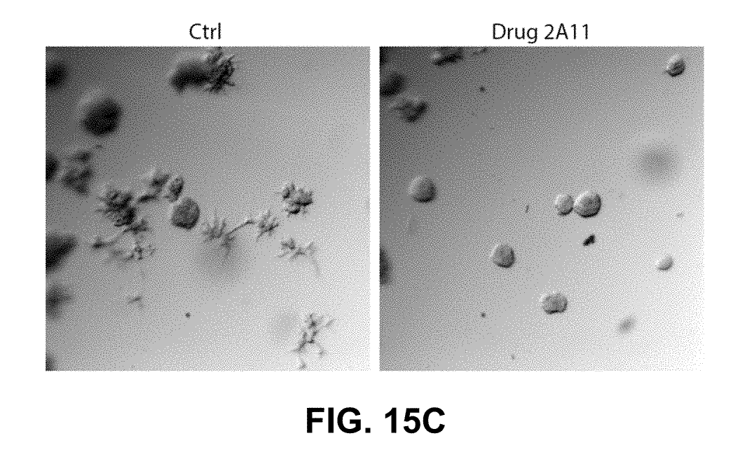

The presently disclosed subject matter also makes it possible to expand cells, both from tumors and normal breast tissues, while retaining expression of hormone receptors. FIG. 15A shows that patient-derived mammary cells expanded for 5 days in our hydrogels retain expression of the estrogen (ER) and progesterone (PR) receptors. Moreover, we show that inhibition of a kinase that is known to be important for breast cancer cell invasion blocks the invasion of patient-derived breast cells in our hydrogels (FIG. 15B). These findings establish that it is possible to assess the function of chemical agents in our hydrogels.

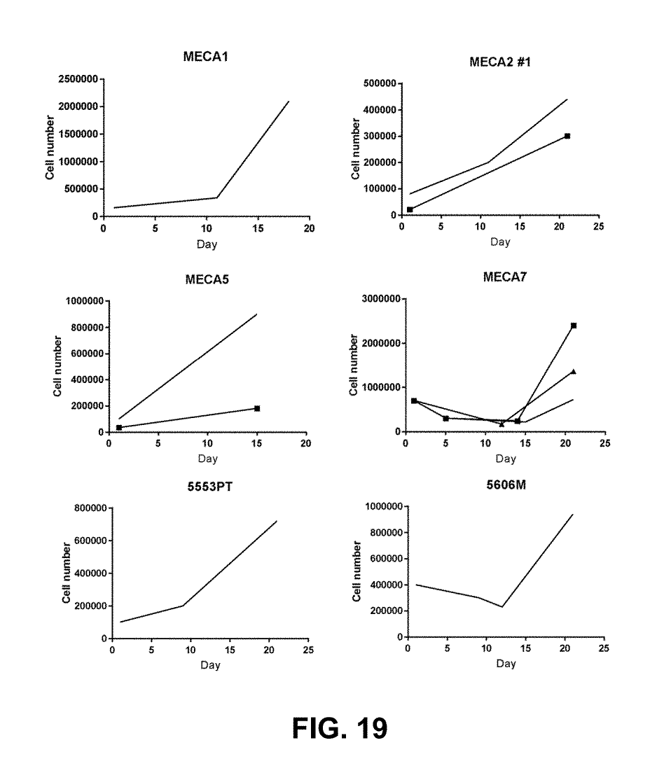

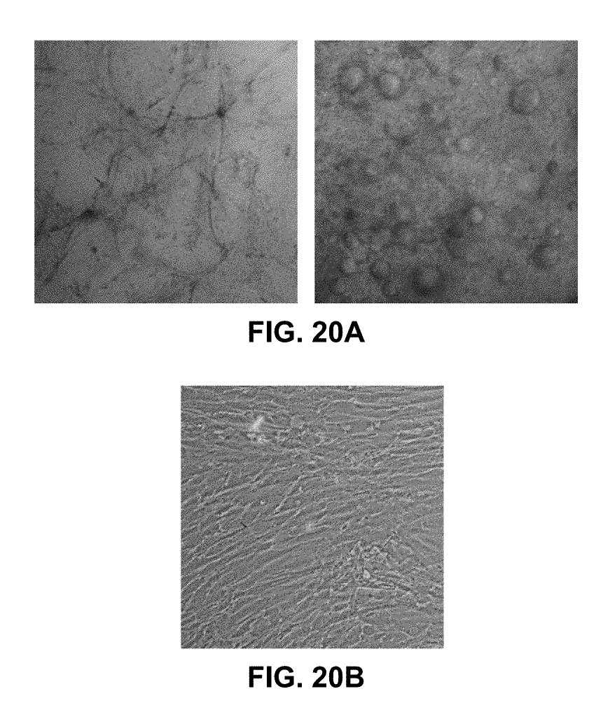

The hydrogel culture system describe herein led to a high success rate of expanding breast cancer tissues in vitro. Over 70% of breast cancer tissue samples tested (6 independent samples) were successfully maintained and expanded using our hydrogel culture system (see, e.g., FIG. 19). In addition to maintaining their growth, the hydrogel culture also faithfully preserved the essential characteristics of these cancerous tissues. For instance, invasive carcinoma-derived breast cancer tissues exhibited highly invasive phenotype in the hydrogel culture (FIG. 20A, left panel). In contrast, non-invasive cancer-derived cancer tissues (from an in situ lobular tumor) mostly maintained a benign and encapsulated growth (FIG. 20A, right panel). No cancer cells grew out if the patient-derived tissue from the same source as in FIG. 20A was cultured in traditional 2D conditions (FIG. 20B). Instead, only cells with a fibroblast morphology were produced. FIGS. 20A and 20B show that morphologies of breast cancer samples grown in 3D culture resemble the descriptions in the pathology report on the cancers from which the samples were obtained, whereas samples cultured in 2D culture (2D tissue-culture treated plastic dishes using the same as the media used for the 3D culture do not. The left panel of FIG. 20A is an image showing that an invasive carcinoma grew as scattered cells in culture. FIG. 20B shows that a breast cancer sample obtained from the same cancer as the sample shown in the right panel of FIG. 20A only produced cells with a fibroblast morphology when cultured in 2D culture. Shown is an image at 2 weeks after seeding. Culture media used in these experiments was a 1:1 mixture of OptiMEM (Gibco REF 31985-070) and MEGM (Lonza CC-3151 & CC-4136).

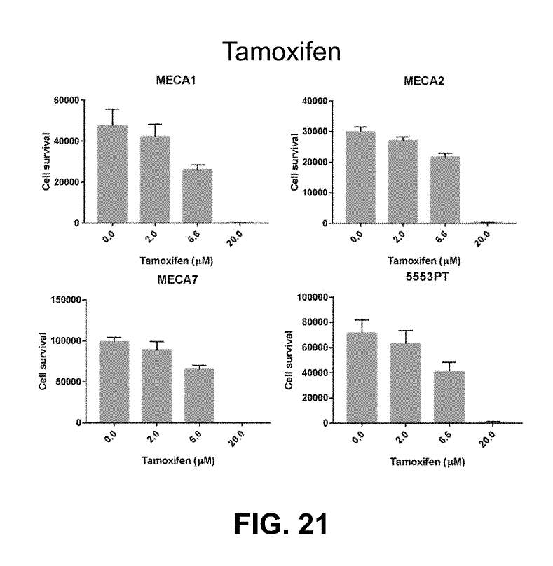

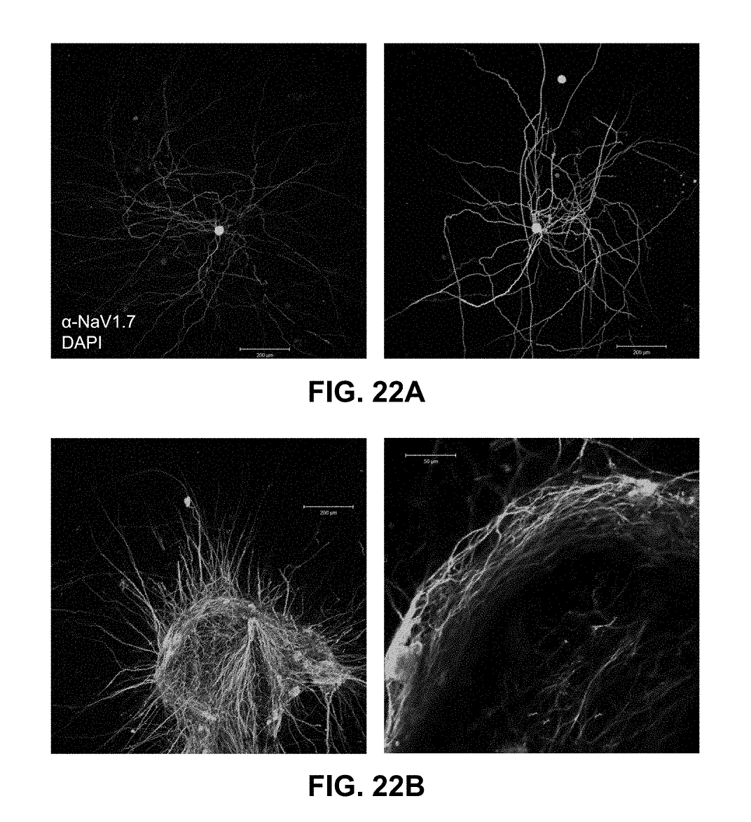

As another illustration of the presently disclosed subject matter, FIG. 21 shows that the 3D hydrogel culture maintained the responsiveness of cancer cells to chemotherapy, since all ER-positive cancer cells were consistently responsive to estrogen receptor (ER) inhibition by tamoxifen treatment.

As another proof-of-concept, we have screened a library of −700 FDA-approved drugs against cancer cells seeded into 3D, and using time-lapse microscopy to score for chemicals that blocked or reversed cancer spheroid invasion. Using this approach, we were able to find drugs that are not currently used for cancer treatment (but are FDA-approved for other conditions) that can, in principle, be repurposed to target tumor invasion.

### Example 4

### Growth of Primary Melanoma Cells in 3D Hydrogel Scaffolds

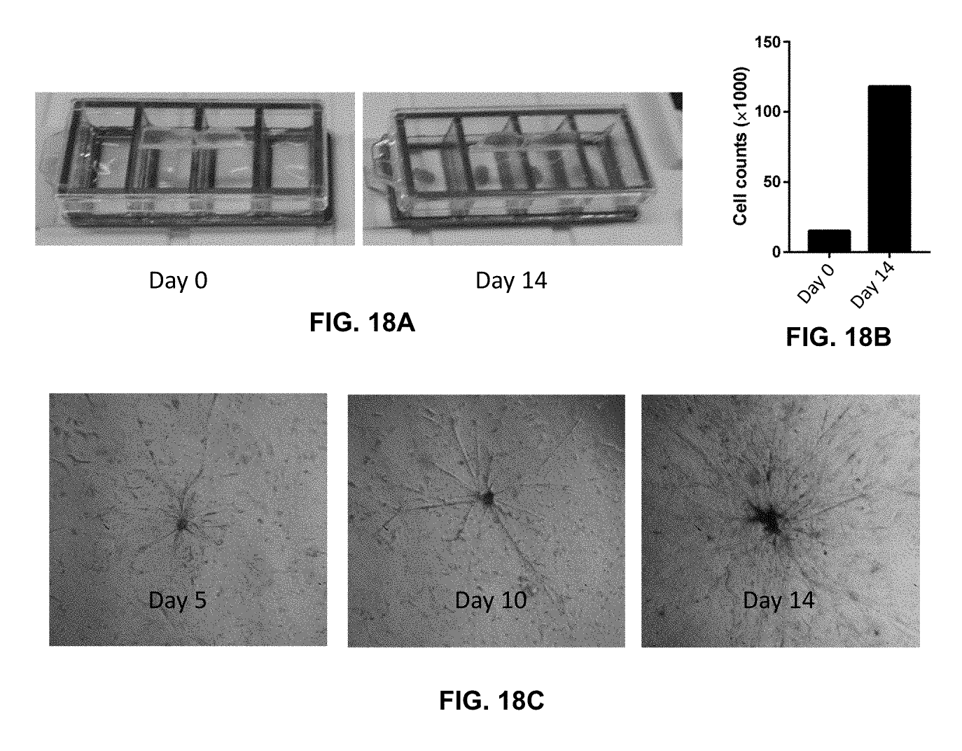

Using the hydrogel culture system described herein, we performed 3D culture on primary tumor samples derived from melanoma patients. The hydrogel culture led to a high success rate of expanding melanoma tissues in vitro. 100% of melanoma samples tested (3 independent samples) were successfully maintained and expanded using our hydrogel culture system (FIG. 18). In addition to maintaining their growth, the hydrogel culture also faithfully preserved the essential characteristics of these cancerous tissues. For instance, the melanoma tissues continued to express high level of melanin (FIG. 18).

The hydrogels used to culture cells derived from melanomas in this example were prepared as described in Example 1 (see Supporting Methods above). The culture medium used was a 1:1 mixture of MEGM and OptiMEM. In other experiments, melanoma tissues were successfully cultured in MEGM.

### Example 5

### Growth of Primary Neurons in 3D Hydrogel Scaffolds

We have demonstrated that mouse neurons seeded into our hydrogels survive and grow new axons and dendrites upon treatment with nerve growth factor (FIG. 22). Mouse dorsal root ganglia were collected and either dissociated into single cells (FIG. 22A) or kept intact (FIG. 22B), and seeded into hydrogels containing no growth factors aside from 40 ng/mL nerve growth factor (NGF). They were cultured in 50% DMEM, 50% F12 growth media supplemented with 10% heat-inactivated fetal bovine serum and 40 ng/mL NGF. The cultures were grown for nine days, at which point they were fixed and stained for the expression of the sodium channel NaV1.7 (a marker of nociceptive neurons) and the DNA dye DAPI.

The hydrogel culture system may be used to culture neurons (e.g., primary neurons) and screen for compounds that, for example, either potentiate or inhibit neuronal activity, e.g., in pre-clinical drug development for neurological diseases.

### REFERENCES

All publications, patent applications, patents, and other references mentioned in the specification are indicative of the level of those skilled in the art to which the presently disclosed subject matter pertains. All publications, patent applications, patents, and other references (e.g., websites, databases, etc.) mentioned in the specification are herein incorporated by reference in their entirety to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Barcellos-Hoff, M. H., Aggeler, J., Ram, T. G., and Bissell, M. J. (1989). Functional differentiation and alveolar morphogenesis of primary mammary cultures on reconstituted basement membrane. Development 105, 223-235.
Berdichevsky, F., Alford, D., D'Souza, B., and Taylor-Papadimitriou, J. (1994). Branching morphogenesis of human mammary epithelial cells in collagen gels. Journal of cell science 107 (Pt 12), 3557-3568.
Cabernard, C., and Affolter, M. (2005). Distinct roles for two receptor tyrosine kinases in epithelial branching morphogenesis in Drosophila. Developmental cell 9, 831-842.
Cardiff, R. D., and Wellings, S. R. (1999). The comparative pathology of human and mouse mammary glands. Journal of mammary gland biology and neoplasia 4, 105-122.
Chen, Q., Zhang, N., Gray, R. S., Li, H., Ewald, A. J., Zahnow, C. A., and Pan, D. (2014). A temporal requirement for Hippo signaling in mammary gland differentiation, growth, and tumorigenesis. Genes & development 28, 432-437.
Debnath, J., Muthuswamy, S. K., and Brugge, J. S. (2003). Morphogenesis and oncogenesis of MCF-10A mammary epithelial acini grown in three-dimensional basement membrane cultures. Methods 30, 256-268.
Dhimolea, E., Maffini, M. V., Soto, A. M., and Sonnenschein, C. (2010). The role of collagen reorganization on mammary epithelial morphogenesis in a 3D culture model. Biomaterials 31, 3622-3630.
Emerman, J. T., Enami, J., Pitelka, D. R., and Nandi, S. (1977). Hormonal effects on intracellular and secreted casein in cultures of mouse mammary epithelial cells on floating collagen membranes. Proceedings of the National Academy of Sciences of the United States of America 74, 4466-4470.
Emerman, J. T., and Pitelka, D. R. (1977). Maintenance and induction of morphological differentiation in dissociated mammary epithelium on floating collagen membranes. In vitro 13, 316-328.
Ewald, A. J., Brenot, A., Duong, M., Chan, B. S., and Werb, Z. (2008). Collective epithelial migration and cell rearrangements drive mammary branching morphogenesis. Developmental cell 14, 570-581.
Fata, J. E., Werb, Z., and Bissell, M. J. (2004). Regulation of mammary gland branching morphogenesis by the extracellular matrix and its remodeling enzymes. Breast cancer research: BCR 6, 1-11.
Gattazzo, F., Urciuolo, A., and Bonaldo, P. (2014). Extracellular matrix: a dynamic microenvironment for stem cell niche. Biochimica et biophysica acta 1840, 2506-2519.
Gerhardt, H., Golding, M., Fruttiger, M., Ruhrberg, C., Lundkvist, A., Abramsson, A., Jeltsch, M., Mitchell, C., Alitalo, K., Shima, D., et al. (2003). VEGF guides angiogenic sprouting utilizing endothelial tip cell filopodia. The Journal of cell biology 161, 1163-1177.
Gudjonsson, T., Villadsen, R., Nielsen, H. L., Ronnov-Jessen, L., Bissell, M. J., and Petersen, O. W. (2002). Isolation, immortalization, and characterization of a human breast epithelial cell line with stem cell properties. Genes & development 16, 693-706.
Guo, W., Keckesova, Z., Donaher, J. L., Shibue, T., Tischler, V., Reinhardt, F., Itzkovitz, S., Noske, A., Zurrer-Hardi, U., Bell, G., et al. (2012). Slug and Sox9 cooperatively determine the mammary stem cell state. Cell 148, 1015-1028.
Gupta, P. B., Kuperwasser, C., Brunet, J. P., Ramaswamy, S., Kuo, W. L., Gray, J. W., Naber, S. P., and Weinberg, R. A. (2005). The melanocyte differentiation program predisposes to metastasis after neoplastic transformation. Nature genetics 37, 1047-1054.
Hadden, J. W., Galy, A., Chen, H., and Hadden, E. M. (1989). A pituitary factor induces thymic epithelial cell proliferation in vitro. Brain, behavior, and immunity 3, 149-159.
Hennighausen, L., and Robinson, G. W. (2001). Signaling pathways in mammary gland development. Developmental cell 1, 467-475.
Hisha, H., Tanaka, T., Kanno, S., Tokuyama, Y., Komai, Y., Ohe, S., Yanai, H., Omachi, T., and Ueno, H. (2013). Establishment of a novel lingual organoid culture system: generation of organoids having mature keratinized epithelium from adult epithelial stem cells. Scientific reports 3, 3224.
Honeth, G., Schiavinotto, T., Vaggi, F., Marlow, R., Kanno, T., Shinomiya, I., Lombardi, S., Buchupalli, B., Graham, R., Gazinska, P., et al. (2015). Models of breast morphogenesis based on localization of stem cells in the developing mammary lobule. Stem cell reports 4, 699-711.
Lancaster, M. A., Renner, M., Martin, C. A., Wenzel, D., Bicknell, L. S., Hurles, M. E., Homfray, T., Penninger, J. M., Jackson, A. P., and Knoblich, J. A. (2013). Cerebral organoids model human brain development and microcephaly. Nature 501, 373-379.
Lee, E. Y., Lee, W. H., Kaetzel, C. S., Parry, G., and Bissell, M. J. (1985). Interaction of mouse mammary epithelial cells with collagen substrata: regulation of casein gene expression and secretion. Proceedings of the National Academy of Sciences of the United States of America 82, 1419-1423.
Lee, E. Y., Parry, G., and Bissell, M. J. (1984). Modulation of secreted proteins of mouse mammary epithelial cells by the collagenous substrata. The Journal of cell biology 98, 146-155.
Lin, D. C., Dimitriadis, E. K., and Horkay, F. (2007). Robust strategies for automated AFM force curve analysis—I. Non-adhesive indentation of soft, inhomogeneous materials. Journal of biomechanical engineering 129, 430-440.
Linnemann, J. R., Miura, H., Meixner, L. K., Irmler, M., Kloos, U. J., Hirschi, B., Bartsch, H. S., Sass, S., Beckers, J., Theis, F. J., et al. (2015). Quantification of regenerative potential in primary human mammary epithelial cells. Development 142, 3239-3251.
Lu, P., and Werb, Z. (2008). Patterning mechanisms of branched organs. Science 322, 1506-1509.
Mani, S. A., Guo, W., Liao, M. J., Eaton, E. N., Ayyanan, A., Zhou, A. Y., Brooks, M., Reinhard, F., Zhang, C. C., Shipitsin, M., et al. (2008). The epithelial-mesenchymal transition generates cells with properties of stem cells. Cell 133, 704-715.
McCracken, K. W., Cata, E. M., Crawford, C. M., Sinagoga, K. L., Schumacher, M., Rockich, B. E., Tsai, Y. H., Mayhew, C. N., Spence, J. R., Zavros, Y., et al. (2014). Modelling human development and disease in pluripotent stem-cell-derived gastric organoids. Nature 516, 400-404.
Mills, E. S., and Topper, Y. J. (1969). Mammary alveolar epithelial cells: effect of hydrocortisone on ultrastructure. Science 165, 1127-1128.
Pasic, L., Eisinger-Mathason, T. S., Velayudhan, B. T., Moskaluk, C. A., Brenin, D. R., Macara, L G., and Lannigan, D. A. (2011). Sustained activation of the HER1-ERK1/2-RSK signaling pathway controls myoepithelial cell fate in human mammary tissue. Genes & development 25, 1641-1653.
Paszek, M. J., Zahir, N., Johnson, K. R., Lakins, J. N., Rozenberg, G. I., Gefen, A., Reinhart-King, C. A., Margulies, S. S., Dembo, M., Boettiger, D., et al. (2005). Tensional homeostasis and the malignant phenotype. Cancer cell 8, 241-254.
Perez-Castro, C., Renner, U., Haedo, M. R., Stalla, G. K., and Arzt, E. (2012). Cellular and molecular specificity of pituitary gland physiology. Physiological reviews 92, 1-38.
Rios, A. C., Fu, N. Y., Lindeman, G. J., and Visvader, J. E. (2014). In situ identification of bipotent stem cells in the mammary gland. Nature 506, 322-327.
Sato, T., Vries, R. G., Snippert, H. J., van de Wetering, M., Barker, N., Stange, D. E., van Es, J. H., Abo, A., Kujala, P., Peters, P. J., et al. (2009). Single Lgr5 stem cells build crypt-villus structures in vitro without a mesenchymal niche. Nature 459, 262-265.
Schedin, P., and Keely, P. J. (2011). Mammary gland ECM remodeling, stiffness, and mechanosignaling in normal development and tumor progression. Cold Spring Harbor perspectives in biology 3, a003228.
Shackleton, M., Vaillant, F., Simpson, K. J., Stingl, J., Smyth, G. K., Asselin-Labat, M. L., Wu, L., Lindeman, G. J., and Visvader, J. E. (2006). Generation of a functional mammary gland from a single stem cell. Nature 439, 84-88.
Simian, M., Hirai, Y., Navre, M., Werb, Z., Lochter, A., and Bissell, M. J. (2001). The interplay of matrix metalloproteinases, morphogens and growth factors is necessary for branching of mammary epithelial cells. Development 128, 3117-3131.
Smalley, M., and Ashworth, A. (2003). Stem cells and breast cancer: A field in transit. Nature reviews Cancer 3, 832-844.
Sokol, E. S., Sanduj a, S., Jin, D. X., Miller, D. H., Mathis, R. A., and Gupta, P. B. (2015). Perturbation-expression analysis identifies RUNX1 as a regulator of human mammary stem cell differentiation. PLoS computational biology 11, e1004161.
Srinivasan, K., Strickland, P., Valdes, A., Shin, G. C., and Hinck, L. (2003). Netrin-1/neogenin interaction stabilizes multipotent progenitor cap cells during mammary gland morphogenesis. Developmental cell 4, 371-382.
Sternlicht, M. D., Sunnarborg, S. W., Kouros-Mehr, H., Yu, Y., Lee, D. C., and Werb, Z. (2005). Mammary ductal morphogenesis requires paracrine activation of stromal EGFR via ADAM17-dependent shedding of epithelial amphiregulin. Development 132, 3923-3933.
Stingl, J., Eirew, P., Ricketson, I., Shackleton, M., Vaillant, F., Choi, D., Li, H. I., and Eaves, C. J. (2006). Purification and unique properties of mammary epithelial stem cells. Nature 439, 993-997.
Takasato, M., Er, P. X., Becroft, M., Vanslambrouck, J. M., Stanley, E. G., Elefanty, A. G., and Little, M. H. (2014). Directing human embryonic stem cell differentiation towards a renal lineage generates a self-organizing kidney. Nature cell biology 16, 118-126.
Tanos, T., Sflomos, G., Echeverria, P. C., Ayyanan, A., Gutierrez, M., Delaloye, J. F., Raffoul, W., Fiche, M., Dougall, W., Schneider, P., et al. (2013). Progesterone/RANKL is a major regulatory axis in the human breast. Science translational medicine 5, 182ra155.
Visvader, J. E. (2009). Keeping abreast of the mammary epithelial hierarchy and breast tumorigenesis. Genes & development 23, 2563-2577.
Weber, K., Thomaschewski, M., Warlich, M., Volz, T., Cornils, K., Niebuhr, B., Tager, M., Lutgehetmann, M., Pollok, J. M., Stocking, C., et al. (2011). RGB marking facilitates multicolor clonal cell tracking. Nature medicine 17, 504-509.
Wiseman, B. S., and Werb, Z. (2002). Stromal effects on mammary gland development and breast cancer. Science 296, 1046-1049.
Wozniak, M. A., Desai, R., Solski, P. A., Der, C. J., and Keely, P. J. (2003). ROCK-generated contractility regulates breast epithelial cell differentiation in response to the physical properties of a three-dimensional collagen matrix. The Journal of cell biology 163, 583-595.
Yang, J., Balakrishnan, A., Hamamoto, S., Elias, J. J., Rosenau, W., Beattie, C. W., Das Gupta, T. K., Wellings, S. R., and Nandi, S. (1987). Human breast epithelial cells in serum-free collagen gel primary culture: growth, morphological, and immunocytochemical analysis. Journal of cellular physiology 133, 228-234, 254-225.
Yang, J., Richards, J., Guzman, R., Imagawa, W., and Nandi, S. (1980). Sustained growth in primary culture of normal mammary epithelial cells embedded in collagen gels. Proceedings of the National Academy of Sciences of the United States of America 77, 2088-2092.

Although the foregoing subject matter has been described in some detail by way of illustration and example for purposes of clarity of understanding, it will be understood by those skilled in the art that certain changes and modifications can be practiced within the scope of the appended claims.

1. A hydrogel precursor solution for forming a three-dimensional hydrogel that supports growth of a physiologically relevant tissue when at least one cell is cultured in the three-dimensional hydrogel, the hydrogel precursor solution consisting of, consisting essentially of, or comprising:
(a) an aqueous medium;
(b) at least three hydrogel precursor components dissolved in the aqueous medium to form a hydrogel precursor solution for forming a three-dimensional hydrogel that supports growth of physiologically relevant tissue, wherein the at least three hydrogel precursor components comprise:
(i) a first hydrogel precursor component comprising an extracellular matrix protein selected from the group consisting of collagen, fibronectin, and laminin;
(ii) a second hydrogel precursor component comprising hyaluronan or a glycosaminoglycan having a water-chelating ability that is similar to hyaluronan; and
(ii) a third hydrogel precursor component comprising at least one agent that promotes growth of a physiologically relevant tissue,
wherein the hydrogel precursor solution polymerizes under suitable conditions to form a three-dimensional hydrogel that supports growth of a physiologically relevant tissue when at least one cell is cultured in the three-dimensional hydrogel.

2. (canceled)

3. A method of forming a three-dimensional hydrogel that supports growth of a physiologically relevant tissue when at least one cell is cultured in the three-dimensional hydrogel, the method comprising:
(a) providing the hydrogel precursor solution of claim 1; and
(b) incubating the hydrogel precursor solution at an elevated temperature for a period of time sufficient for the hydrogel precursor solution to polymerize and form a three-dimensional hydrogel that supports growth of a physiologically relevant tissue when at least one cell is cultured in the three-dimensional hydrogel.

4. A three-dimensional hydrogel that supports growth of a physiologically relevant tissue when at least one cell is cultured in the three-dimensional hydrogel, the three-dimensional hydrogel consisting of, consisting essentially of, or comprising:
(a) an extracellular matrix protein selected from the group consisting of collagen, fibronectin, and laminin;
(b) hyaluronan or a glycosaminoglycan having a water-chelating ability that is similar to hyaluronan; and
(c) at least one agent that promotes growth of a physiologically relevant tissue; and
(d) at least one cell,
wherein (a) and (b) are polymerized into a three-dimensional hydrogel and (c) and (d) are embedded in the three-dimensional hydrogel; and
wherein the three-dimensional hydrogel supports growth of a physiologically relevant tissue when (d) is cultured in the three-dimensional hydrogel in the presence of (c).

5. A method for growing a physiologically relevant tissue from at least one cell, the method consisting of, consisting essentially of, or comprising:
(a) providing the three-dimensional hydrogel of claim 4;
(b) optionally providing a defined culture medium; and
(c) culturing the at least one cell in the three-dimensional hydrogel, in the presence of the defined culture medium if provided, for a period of time sufficient for the at least one cell to grow into a physiologically relevant tissue or physiologically relevant component thereof.

6.-10. (canceled)

11. The solution, kit, hydrogel, physiologically relevant tissue, or method according to claim 1, wherein the collagen is present at a concentration of between 0.5 mg/ml and 4.0 mg/ml.

12. The solution, kit, hydrogel, physiologically relevant tissue, or method according to claim 11, wherein the fibronectin is present at a concentration of between 1 μg/mL and 50 μg/mL.

13. The solution, kit, hydrogel, physiologically relevant tissue, or method according to claim 12, wherein the laminin is present at a concentration of between 20 μg/ml and 60 μg/ml.

14. (canceled)

15. The solution, kit, hydrogel, physiologically relevant tissue, or method according to claim 13, wherein the hyaluronan is present at a concentration of between 1 μg/mL and 50 μg/mL.

16. (canceled)

17. The solution, kit, hydrogel, physiologically relevant tissue, or method according to claim 1, wherein the hyaluronan comprising a low molecular weight hyaluronic acid and a high molecular weight hyaluronic acid.

18.-19. (canceled)

20. The solution, kit, hydrogel, physiologically relevant tissue, or method according to claim 1, wherein the physiologically relevant tissue comprises epithelium.

21-50. (canceled)

51. The solution, kit, hydrogel, physiologically relevant tissue, or method according to claim 4, wherein the at least one cell comprises at least one mammary cell and is cultured in the three-dimensional hydrogel in a culture medium that comprises at least one agent that stimulates development of mammary tissue in vivo.

52.-58. (canceled)

59. The solution, kit, hydrogel, physiologically relevant tissue, or method according to claim 4, wherein the at least one cell comprises at least one mammary epithelial cell, or at least one cluster of mammary epithelial cells, and wherein when the at least one mammary epithelial cell, or at least one cluster of mammary epithelial cells, is cultured in the three-dimensional hydrogel, the at least one mammary epithelial cell, or at least one cluster of mammary epithelial cells, grows into physiologically relevant mammary tissue in the three-dimensional hydrogel.

60. The solution, kit, hydrogel, physiologically relevant tissue, or method according to claim 59, wherein during growth of the physiologically relevant mammary tissue, the cultured cells and/or growing physiologically relevant mammary tissue exhibits at least one of the following features:
i) ductal initiation and/or ductal elongation;
ii) a tip at a leading edge of at least one elongating duct, wherein the tip comprises one or two leader cells polarized in the direction of ductal elongation;
iii) leader cells expressing basal cytokeratins, staining positively for filamentous actin, and co-expressing SLUG and SOX9;
iv) organization into expanding tissues comprising an outer CK14+ basal layer and interior CK8/18+ luminal cells;
v) lobule interiors expressing luminal lineage marker GATA3, and luminal differentiation marker MUC1;
vi) cavitation of lobule interiors;
vii) secondary and tertiary ductal branching selected from the group consisting of bifurcated elongated ducts and side-branches sprouted from primary ducts;
viii) lipid droplets;
ix) hormone-responsiveness;
x) terminal ductal-lobular units (TDLUs), wherein at least a portion of the cells comprising the TDLUs are SLUG+/SOX9+ mammary stem cells;
xi) TDLUs comprising layers of between 5 and 8 cells; and
xii) expression of hormone receptors selected from the group consisting of estrogen receptors, progesterone receptors, glucocorticoid receptors, and androgen receptors.

61.-65. (canceled)

66. A method for producing hormone-responsive, milk-producing mammary tissue, the method comprising culturing at least one mammary epithelial cell or at least one cluster of mammary epithelial cells in the three-dimensional hydrogel according to claim 4 in the presence of at least one agent that stimulates development of mammary tissue in vivo for a sufficient amount of time to produce hormone-responsive, milk-producing mammary tissue.

67.-87. (canceled)

88. A method of evaluating the effect of an agent on a biological condition of cells, the method comprising:
(a) providing a three-dimensional hydrogel according to claim 4;
(b) culturing at least one cell or at least one cluster of cells in the three-dimensional hydrogel for a period of time sufficient for the at least one cell or at least one cluster of cells to expand in the three-dimensional hydrogel; and
(c) exposing the expanding cells in the three-dimensional hydrogel to an agent; and
(d) evaluating the effect of the agent on the biological condition of the cells.

89. A method of evaluating the effect of an agent on a biological condition of a physiologically relevant tissue, the method comprising:
(a) providing a three-dimensional hydrogel according to claim 4;
(b) culturing at least one cell or at least one cluster of cells in the three-dimensional hydrogel for a period of time sufficient for a physiologically relevant tissue to grow in the three-dimensional hydrogel; and
(c) exposing the physiologically relevant tissue growing in the three-dimensional hydrogel to an agent; and
(d) evaluating the effect of the test agent on the biological condition of the physiologically relevant tissue.

90.-93. (canceled)

94. The method according to claim 88, wherein the at least one cell or at least one cluster of cells comprises at least one cancer cell or at least one cluster of cancer cells obtained from a subject.

95.-97. (canceled)

98. The method according to claim 88, wherein evaluating the effect of the agent on the biological condition identifies at least one of a change in growth rate, cell number, cell shape, viability, function, and morphology of the cells.

99.-105. (canceled)

106. The method according to claim 88, wherein the agent is a candidate therapeutic agent.

107. The method according to claim 106, wherein the candidate therapeutic agent is a candidate chemotherapeutic agent.

108.-112. (canceled)

113. A method of screening for a candidate chemotherapeutic agent, the method comprising:
(a) culturing at least one cancer cell in a three-dimensional hydrogel according to claim 4 for a sufficient amount of time for growth of the at least one cancer cell in the three-dimensional hydrogel to occur;
(b) exposing the at least one cancer cell in the three-dimensional hydrogel to at least one test agent; and
(c) measuring growth of the at least one cancer cell in the three-dimensional hydrogel in the presence of the test agent, wherein a decrease in growth of the at least one cancer cell in the presence of the test agent as compared to a control identifies the agent as a candidate chemotherapeutic agent.

114.-119. (canceled)

120. The method according to claim 113, wherein the at least one cancer cell is obtained by dissociating tumor tissue obtained from a subject into single cells.

121.-133. (canceled)

134. The method according to claim 113, wherein the at least one cancer cell is exposed to multiple test agents in the three-dimensional hydrogel.

135.-137. (canceled)

138. An immunocompromised animal comprising a three-dimensional hydrogel, or a hydrogel precursor solution thereof, according to claim 4 implanted into it.

139.-142. (canceled)

143. The animal of claim 138, wherein the three-dimensional hydrogel comprises a patient tumor xenograft comprising at least one cell obtained from a patient suffering from a disease, wherein the at least one cell is dissociated from a patient's diseased tissue and is cultured in the three-dimensional hydrogel, wherein the at least one cell comprises at least one cancer cell.

144.-146. (canceled)

147. The solution, kit, hydrogel, physiologically relevant tissue, or method according to claim 4, wherein the physiologically relevant tissue comprises non-epithelial tissue.

148.-162. (canceled)

163. A method for expanding cancer cells from a patient, the method comprising:
(a) providing the three-dimensional hydrogel of claim 4, wherein the at least one cell comprises at least one cancer cell from a patient;
(b) optionally providing a defined culture medium; and
(c) culturing the at least one cell in the three-dimensional hydrogel, in the presence of the defined culture medium if provided, for a period of time sufficient for the at least one cancer cell to expand.

164. The method of claim 163, wherein the at least one cell was obtained by dissociating a tumor sample from a patient into single cells.

165. A method for expanding a patient's cancer cells in culture, the method comprising:
(a) providing one or more cancer cells obtained by dissociating a tumor sample from a patient into single cells;
(b) seeding the one or more cells into a three-dimensional (3D) hydrogel scaffold comprising polymerized collagen, hyaluronan, and at least one agent that promotes growth of a physiologically relevant tissue; and
(c) maintaining the 3D hydrogel scaffold in culture for a sufficient period of time for the one or more cancer cells to expand.

166. The method of claim 163, wherein the 3D hydrogel comprises polymerized collagen, hyaluronan, fibronectin, and laminin.
